SEQUENCE LISTING (57 sequences)

SEQ ID NO: 1 - protein, 10 aa, synthetic construct
NGAAALWGKR

SEQ ID NO: 2 - protein, 4 aa, synthetic construct
LWGK

SEQ ID NO: 3 - protein, 13 aa, synthetic construct
  VARIANT 9: note = X is any amino acid
WGGGSGASXSGAS

SEQ ID NO: 4 - protein, 15 aa, synthetic construct
  VARIANT 9..11: note = X can be any amino acid
WGGGSGASXXXSGAS

SEQ ID NO: 5 - protein, 10 aa, synthetic construct
SESSKGNLYY

SEQ ID NO: 6 - protein, 10 aa, synthetic construct
  VARIANT 4: note = X can be any amino acid
SESXXXNLYY

SEQ ID NO: 7 - protein, 10 aa, synthetic construct
  VARIANT 4..6: note = X can be any amino acid
SESXXXNLYY

SEQ ID NO: 8 - protein, 13 aa, synthetic construct
  VARIANT 9: note = X can be any amino acid
  VARIANT 4: note = X can be any amino acid
SESXSGASXNLYY

SEQ ID NO: 9 - protein, 15 aa, synthetic construct
  VARIANT 11: note = X can be any amino acid
SESWFRSGASXNLYY

SEQ ID NO: 10 - protein, 17 aa, synthetic construct
  VARIANT 11..13: note = X can be any amino acid
SESWFRSGASXXXNLYY

SEQ ID NO: 11 - protein, 10 aa, synthetic construct
NDFSQTTISR

SEQ ID NO: 12 - protein, 8 aa, synthetic construct
NDFZTISR

SEQ ID NO: 13 - protein, 10 aa, synthetic construct
NDFXXXTISR

SEQ ID NO: 14 - protein, 13 aa, synthetic construct
  VARIANT 4: note = X can be any amino acid
  VARIANT 9: note = X can be any amino acid
NDFXSGASXTISR

SEQ ID NO: 15 - protein, 15 aa, synthetic construct
  VARIANT 11: note = X can be any amino acid
NDFWFRSGASXTISR

SEQ ID NO: 16 - protein, 17 aa, synthetic construct
  VARIANT 11..13: note = X can be any amino acid
NDFWFRSGASXXXTISR

SEQ ID NO: 17 - protein, 8 aa, synthetic construct
  VARIANT 4: note = X can be any amino acid
SGSXNLYY

SEQ ID NO: 18 - protein, 10 aa, synthetic construct
  VARIANT 4..6: note = X can be any amino acid
SGSXXXNLYY

SEQ ID NO: 19 - protein, 13 aa, synthetic construct
  VARIANT 4: note = X can be any amino acid
  VARIANT 9: note = X can be any amino acid
SGSXSGASXNLYY

SEQ ID NO: 20 - protein, 15 aa, synthetic construct
  VARIANT 11: note = X can be any amino acid
SGSWFRSGASXNLYY

SEQ ID NO: 21 - protein, 17 aa, synthetic construct
  VARIANT 11..13: note = X can be any amino acid
SGSWFRSGASXXXNLYY

SEQ ID NO: 22 - protein, 8 aa, synthetic construct
  VARIANT 4: note = X can be any amino acid
NGFXTISR

SEQ ID NO: 23 - protein, 10 aa, synthetic construct
  VARIANT 4..6: note = X can be any amino acid
NGFXXXTISR

SEQ ID NO: 24 - protein, 13 aa, synthetic construct
  VARIANT 4: note = X can be any amino acid
NGFXSGASXTISR

SEQ ID NO: 25 - protein, 15 aa, synthetic construct
  VARIANT 11: note = X can be any amino acid
NGFWFRSGASXTISR

SEQ ID NO: 26 - protein, 17 aa, synthetic construct
  VARIANT 11..13: note = X can be any amino acid
NGFWFRSGASXXXTISR

SEQ ID NO: 27 - protein, 15 aa, synthetic construct
WGGGSGASRFFSGAS

SEQ ID NO: 28 - protein, 14 aa, synthetic construct
WGGGNDFRFFSGAS

SEQ ID NO: 29 - protein, 14 aa, synthetic construct
WGGGNGFRFFSGAS

SEQ ID NO: 30 - protein, 14 aa, synthetic construct
WGGGSESRFFSGAS

SEQ ID NO: 31 - protein, 14 aa, synthetic construct
WGGGSGSRFFSGAS

SEQ ID NO: 32 - protein, 15 aa, synthetic construct
WGGGSGASWFRSGAS

SEQ ID NO: 33 - protein, 14 aa, synthetic construct
WGGGNDFWFRSGAS

SEQ ID NO: 34 - protein, 14 aa, synthetic construct
WGGGNGFWFRSGAS

SEQ ID NO: 35 - protein, 14 aa, synthetic construct
WGGGSESWFRSGAS

SEQ ID NO: 36 - protein, 14 aa, synthetic construct
WGGGSGSWFRSGAS

SEQ ID NO: 37 - protein, 15 aa, synthetic construct
WGGGSGASGGGSGAS

SEQ ID NO: 38 - protein, 14 aa, synthetic construct
WGGGNDFGGGSGAS

SEQ ID NO: 39 - protein, 14 aa, synthetic construct
WGGGNGFGGGSGAS

SEQ ID NO: 40 - protein, 14 aa, synthetic construct
WGGGSESGGGSGAS

SEQ ID NO: 41 - protein, 14 aa, synthetic construct
WGGGSGSGGGSGAS

SEQ ID NO: 42 - protein, 10 aa, synthetic construct
SGSRFFNLYY

SEQ ID NO: 43 - protein, 10 aa, synthetic construct
SGSTFTNLYY

SEQ ID NO: 44 - protein, 10 aa, synthetic construct
SGSWFRNLYY

SEQ ID NO: 45 - protein, 10 aa, synthetic construct
NGFRFFTISR

SEQ ID NO: 46 - protein, 10 aa, synthetic construct
NGFTFTTISR

SEQ ID NO: 47 - protein, 10 aa, synthetic construct
NGFWFRTISR

SEQ ID NO: 48 - protein, 13 aa, synthetic construct
  VARIANT 4: note = X can be any amino acid
  VARIANT 9: note = X can be any amino acid
SESXSGASXNLYY

SEQ ID NO: 49 - protein, 13 aa, synthetic construct
  VARIANT 4: note = X can be any amino acid
  VARIANT 9: note = X can be any amino acid
SGSXSGASXNLYY

SEQ ID NO: 50 - protein, 13 aa, synthetic construct
  VARIANT 4: note = X can be any amino acid
  VARIANT 9: note = X can be any amino acid
NDFXSGASXTISR

SEQ ID NO: 51 - protein, 13 aa, synthetic construct
  VARIANT 4: note = X can be any amino acid
  VARIANT 9: note = X can be any amino acid
NGFXSGASXTISR

SEQ ID NO: 52 - protein, 9 aa, synthetic construct
  VARIANT 5: note = X can be any amino acid
SGASXSGAS

SEQ ID NO: 53 - protein, 21 aa, synthetic construct
  VARIANT 15..17: note = X can be any amino acid
SESWFRNLYYSGASXXXNLYY

SEQ ID NO: 54 - protein, 21 aa, synthetic construct
  VARIANT 15..17: note = X can be any amino acid
NDFWFRNLYYSGASXXXTISR

SEQ ID NO: 55 - protein, 21 aa, synthetic construct
  VARIANT 15..17: note = X can be any amino acid
SGSWFRNLYYSGASXXXNLYY

SEQ ID NO: 56 - protein, 21 aa, synthetic construct
  VARIANT 15..17: note = X can be any amino acid
NGFWFRNLYYSGASXXXTISR

SEQ ID NO: 57 - protein, 4 aa, synthetic construct
TISR

(19) United States
(12) Patent Application Publication
(10) Pub. No.: US 2024/0093270 A1
(43) Pub. Date: Mar. 21, 2024

(54) GENERATION AND SELECTION OF AFFINITY REAGENTS

(71) Applicant: Nautilus Subsidiary, Inc., Seattle, WA (US)

(72) Inventors: Aimee SANFORD, San Mateo, CA (US); Ahana DUTTA, San Jose, CA (US); Tanvir SAINI, San Mateo, CA (US); Wade DUGDALE, San Mateo, CA (US); Marina KOVALIOV, San Carlos, CA (US); Kaitlyn BURKE, Belmont, CA (US); Filip BARTNICKI, Redwood City, CA (US); Tural AKSEL, Redwood City, CA (US); Sonal TONAPI, Sunnyvale, CA (US); Parag MALLICK, San Mateo, CA (US)

(21) Appl. No.: 18/448,000

(22) Filed: Aug. 10, 2023

Related U.S. Application Data
(60) Provisional application No. 63/371,307, filed on Aug. 12, 2022.
(60) Provisional application No. 63/375,030, filed on Sep. 8, 2022.

(51) Int. Cl. C12Q 1/6811; C12Q 1/6832; C12Q 1/6876

(52) U.S. Cl. CPC C12Q 1/6811; C12Q 1/6832; C12Q 1/6876

(57) ABSTRACT

The present disclosure provides methods, compositions and apparatus for generating probes having affinity for analytes of interest, such as polypeptides. Also provided are methods, compositions and apparatus for evaluating and characterizing probes.

### CROSS-REFERENCE TO RELATED APPLICATIONS

This application claims priority to U.S. Provisional Application Nos. 63/371,307 filed on Aug. 12, 2022 and 63/375,030 filed on Sep. 8, 2022, each of which is incorporated herein by reference.

### REFERENCE TO SEQUENCE LISTING

This application contains a Sequence Listing, which is being submitted electronically in XML format in accordance with the WIPO Standard ST.26 and is hereby incorporated by reference in its entirety. The XML copy, created Aug. 3, 2023, is named “50109-4012.xml” and is 64,288 bytes in size.

### BACKGROUND OF THE INVENTION

U.S. 63/375,030 provisional includes all subject matter of the U.S. 63/371,307 provisional along with additional subject matter. Nucleic acid aptamers and their derivatives find uses in many life science applications, for example, in proteomics, therapeutics, diagnostics, cell sorting, metal ion detection, pathology, cell surface binding, micro-organism binding, nanotechnology components, affinity purification, drug delivery vehicles, etc. This is due in large part to their antibody-like binding properties, relatively small size, general robustness, and ease of synthesis. Techniques for protein identification and quantification are often based on binding of specific and sensitive affinity reagents to the proteins. As a class of affinity ligands, nucleic acid aptamers display some advantages over traditional antibodies such as prolonged shelf life, low batch to batch variation, little to no immunogenicity, and the flexibility to incorporate chemical modifications for enhanced stability or targeted affinities and specificities.

SELEX (systematic evolution of ligands by exponential enrichment) is typically configured as an iterative process for enriching an initial pool of random nucleic acid aptamers and thereby producing a more focused pool of aptamers (a pool enriched for target-binding aptamers). It is a process, generally, with a relatively low success rate and therefore many scientific groups have published methods of improvement for the process. Despite these efforts, success rates are still relatively low, for example, when compared to the average success of generating antibodies to biological targets. Among the substantial, often time-consuming iterative steps in a SELEX process, selection of target-bound aptamers (often on a solid-phase support) from fluid phase contaminant aptamers, is particularly important since contaminating aptamers can be detrimental to downstream production and use of the desired aptamers. Moreover, aptamers that are produced by SELEX often have substantial affinity for off-target epitopes found in bait molecules that are used as selection targets. In many cases, aptamers that target a desired epitope are lost to the process, for example, being out competed by aptamers that bind to off-target epitopes. Therefore, there is a need for methods, compositions and apparatus that enrich for desired aptamers during SELEX or other processes of making or selecting aptamers. The present disclosure addresses this need and provides other advantages as well.

### SUMMARY OF THE INVENTION

The present disclosure provides a method of selecting at least one affinity reagent. The method can include steps of (a) contacting a solid-phase analyte with a fluid-phase library of different affinity reagents in a vessel, wherein the solid-phase includes a particle to which the analyte is attached, wherein the vessel includes a porous filter that prevents passage of the particle; (b) passing the fluid-phase library of affinity reagents through the porous filter to separate affinity reagents of the library from the solid-phase analyte; (c) transferring the solid-phase analyte from the vessel to a second vessel; and (d) washing the solid-phase analyte in the second vessel to remove further affinity reagents of the library from the solid-phase analyte, whereby at least one of the different affinity reagents remains bound to the solid-phase analyte.

The present disclosure provides a polypeptide having an epitope within a structural motif that facilitates binding of the epitope to an affinity reagent. The polypeptide can include a peptide epitope and a flanking moiety on one or both sides of the epitope. A flanking moiety can provide three-dimensional structure to the polypeptide that orients the epitope to bind an affinity reagent such that the affinity reagent need not interact with the flanking regions or other regions of the polypeptide. In particular configurations, a polypeptide of the present disclosure can include an amino acid sequence selected from the group consisting of SGASZSGAS; SESZNLYY; NDFZTISR; SGSZNLYY; and NGFZTISR, wherein Z designates a region of variable amino acid composition and length. Optionally, Z can function as an epitope for recognized by an affinity reagent.

The present disclosure further provides a complex between an affinity reagent and a polypeptide set forth herein. For example, a complex can include a polypeptide having an amino acid sequence selected from the group consisting of SGASZSGAS (SEQ ID NO: 52); SESZNLYY; NDFZTISR; SGSZNLYY; and NGFZTISR, wherein Z designates a region of variable amino acid composition and length.

Also provided is a method of selecting affinity reagents that selective for a desired polypeptide epitope independent of sequence context of the epitope in a larger polypeptide. A selection method can include steps of (a) contacting a fluid phase with a solid-phase support, wherein the fluid phase includes a plurality of different affinity reagents, wherein the solid-phase support is attached to a target polypeptide, thereby forming a solid-phase complex including the target polypeptide of the solid support bound to a first affinity reagent of the plurality of different affinity reagents; and (b) separating the solid-phase complex from the fluid phase. Optionally, the target polypeptide can have a sequence selected from the group consisting of WGGGSGASZSGAS; SESZNLYY; NDFZTISR; SGSZNLYY; and NGFZTISR, wherein Z designates a region of variable amino acid composition and length.

The present disclosure further provides a method for characterizing affinity reagents including the steps of (a) providing a library of affinity reagents; (b) performing an affinity reagent selection process, including: (i) contacting the library with solid-phase target analytes, thereby forming solid-phase complexes including the target analytes and a subset of the candidate affinity reagents, and (ii) separating the subset of candidate affinity reagents from the library, thereby obtaining a pool of selected affinity reagents; and (c) performing a binding assessment process, including: (i) contacting the selected affinity reagents with test analytes, thereby forming complexes including the test analytes and a subset of the selected affinity reagents, (ii) separating the subset of selected affinity reagents from the pool, thereby obtaining a plurality of candidate affinity reagents, and (iii) detecting candidate affinity reagents of the plurality of candidate affinity reagents.

Also provided is a method of producing probes. The method can include steps of (a) forming a first mixture including a plurality of nucleic acid scaffolds and nucleic acid staples, thereby producing structured nucleic acid particles, the structured nucleic acid particles each including a nucleic acid scaffold and a plurality of nucleic acid staples folded into a nucleic acid origami; (b) separating the structured nucleic acid particles from nucleic acid scaffolds and nucleic acid staples of the first mixture; (c) forming a second mixture including a plurality of functionalized nucleic acids and a plurality of the structured nucleic acid particles, wherein the functionalized nucleic acids include affinity reagents, thereby producing probes, the probes each including a structured nucleic acid particle attached to an affinity reagent via the functionalized oligonucleotide; and (d) separating the probes from functionalized nucleic acids and structured nucleic acid particles of the second mixture.

In some configurations, a method of producing probes can include steps of (a) forming a first mixture including a plurality of nucleic acid scaffolds and nucleic acid staples, thereby producing structured nucleic acid particles, the structured nucleic acid particles each including a nucleic acid scaffold and a plurality of nucleic acid staples folded into a nucleic acid origami; (b) separating the structured nucleic acid particles from nucleic acid scaffolds and nucleic acid staples of the first mixture; (c) forming a second mixture including a plurality of functionalized nucleic acids and a plurality of the structured nucleic acid particles, thereby producing functionalized structured nucleic acid particles, the functionalized structured nucleic acid particles each including a nucleic acid origami hybridized to a functionalized nucleic acid; (d) separating the functionalized structured nucleic acid particles from functionalized nucleic acids and structured nucleic acid particles of the second mixture; (e) forming a third mixture including affinity reagents and the functionalized structured nucleic acid particles, thereby producing probes, the probes each including a structured nucleic acid particle attached to an affinity reagent via a moiety of the functionalized oligonucleotide; and (f) separating the probes from affinity reagents and functionalized structured nucleic acid particles of the third mixture.

### BRIEF DESCRIPTION OF THE DRAWINGS

FIG. 1A shows a flow chart describing an exemplary process for preparing affinity reagents.

FIG. 1B shows a flow chart describing an exemplary process for obtaining an affinity reagent library.

FIG. 1C shows a flowchart describing an exemplary process for selecting affinity reagents that bind target analytes.

FIG. 1D shows a flow chart describing an exemplary process for evaluating binding of affinity reagents to test analytes and characterizing structure of the affinity reagents.

FIG. 1E shows a flow chart describing an exemplary process for preparing affinity reagents for use in an assay.

FIG. 2A shows a diagrammatic representation of a selection process for screening and selecting aptamers.

FIG. 2B shows a diagrammatic representation of a modified selection process for screening and selecting aptamers, the modification including addition of a well plate transfer step where solid-phase supports may be transferred from one well plate to a different well plate in order to remove contaminant affinity reagents.

FIG. 2C shows a diagrammatic representation of a similar process as shown in FIG. 2B with the addition of a shunt whereby PCR amplification is omitted.

FIG. 2D shows a diagrammatic representation of a similar process as shown in FIG. 2B with the addition of a negative selection step.

FIG. 2E shows a similar process as shown in FIG. 2B with the addition of a chemically modified aptamer library.

FIG. 3A shows quantities of aptamers selected from a naïve library using various bait molecules having an RFF epitope or control GGG epitope.

FIG. 3B shows quantities of aptamers selected from an aptamer mixture using various bait molecules having an RFF epitope or control GGG epitope.

FIG. 4A shows quantities of aptamers selected from a naïve library using various bait molecules having a WFR epitope or control GGG epitope.

FIG. 4B shows quantities of aptamers selected from an aptamer mixture using various bait molecules having a WFR epitope or control GGG epitope.

FIG. 5A shows a computed three-dimensional model for a polypeptide having the sequence NGFZTISR (SEQ ID NO: 42).

FIG. 5B shows a computed three-dimensional model for a polypeptide having the sequence SGSTFTNLYY (SEQ ID NO: 43).

FIG. 5C shows a computed three-dimensional model for a polypeptide having the sequence SGSWFRNLYY (SEQ ID NO: 44).

FIG. 6A shows a computed three-dimensional model for a polypeptide having the sequence NGFRFFTISR (SEQ ID NO: 45).

FIG. 6B shows a computed three-dimensional model for a polypeptide having the sequence NGFTFTTISR (SEQ ID NO: 46).

FIG. 6C shows a computed three-dimensional model for a polypeptide having the sequence NGFWFRTISR (SEQ ID NO: 47).

FIGS. 7A-7F show results for libraries of aptamers generated from six rounds of random mutagenesis PCR plotted as the number of point mutants observed in an aptamer sequence (X axis) vs. the number of aptamer sequences that have a particular number of point mutants (Y axis).

FIG. 8A tabulates the proportion of aptamer hits per epitope target (“Target”) obtained from performing SELEX with each of three aptamer libraries (“PCR3”, “PCR5” and “Naïve”) and the “Target Hit Against B1?” column identifies which of the epitope targets did (“Yes”) or did not (“No”) bind to the B1 aptamer.

FIG. 8B tabulates the success rate for target epitopes tested based on aptagenicity resulting from the SELEX performed to obtain the data of FIG. 8A.

FIG. 8C shows five aptamers (from the SELEX performed to obtain the data of FIG. 8A) and the target epitopes to which they bind.

FIGS. 9A-9E show ELONA results for binding between various epitope targets and various aptamers. The various aptamers include the B1 aptamer and variants derived from random mutagenesis of the B1 aptamer.

FIG. 10 shows a plot of percent signal gain for aptamers bound to biotinylated test polypeptides vs. concentration of biotinylated test polypeptides, wherein the bound complexes were captured on streptavidin coated beads and detected using fluorescent intercalating agents, and wherein a positive control aptamer (B1) was detected in separate samples compared to background aptamers (sc2).

FIG. 11 shows a plot of percent signal gain for aptamers bound to biotinylated test polypeptides vs. concentration of biotinylated test polypeptides, wherein the bound complexes were captured on streptavidin coated beads and detected using fluorescent intercalating agents and wherein a positive control aptamer (B1) was detected in the presence of background aptamers (sc2).

FIG. 12 shows a flow chart describing an exemplary process for making nucleic acid origami tiles and attaching affinity reagents and/or labels to the origami tiles.

FIG. 13 shows a flow chart describing an exemplary process for making nucleic acid origami tiles, attaching functionalized nucleic acids to the origami tiles to produce functionalized origami tiles, and attaching affinity reagents and/or labels to the functionalized origami tiles.

FIG. 14 shows a process for making labeled aptamer probes.

FIG. 15 shows a high throughput process for making labeled aptamer probes.

### DETAILED DESCRIPTION

The present disclosure provides methods, compositions and apparatus for generating affinity reagents. In particular configurations, the methods, compositions and apparatus can be used to screen candidate affinity reagents and select one or more target affinity reagents having desirable affinity for a target analyte or class of target analytes. Screening can be carried out for large and diverse libraries of candidate affinity reagents. For example, particular aspects of the methods, compositions and apparatus set forth herein can provide valuable improvements for generating aptamers, for example, providing modifications to SELEX (systematic evolution of ligands by exponential enrichment) procedures. Processes used to generate aptamers and other affinity reagents can be automated and carried out in multiwell plate formats to support processing of large and diverse libraries of candidate affinity reagents and, if desired, to support screening of the libraries for ability to bind a large number and variety of target analytes. However, the methods, compositions and apparatus are also suitable for screening small libraries or even evaluating affinity reagents individually.

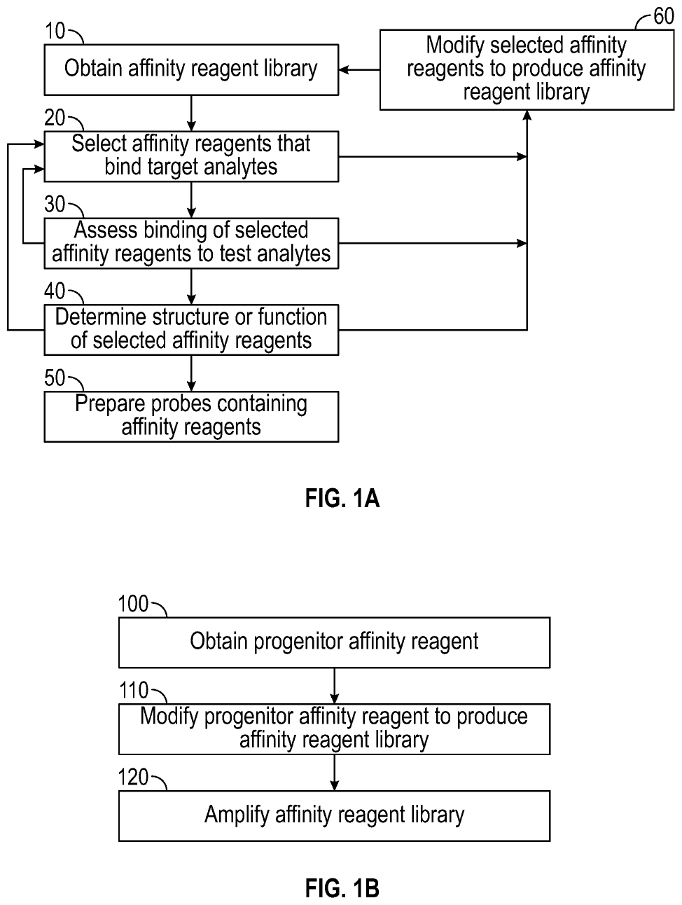

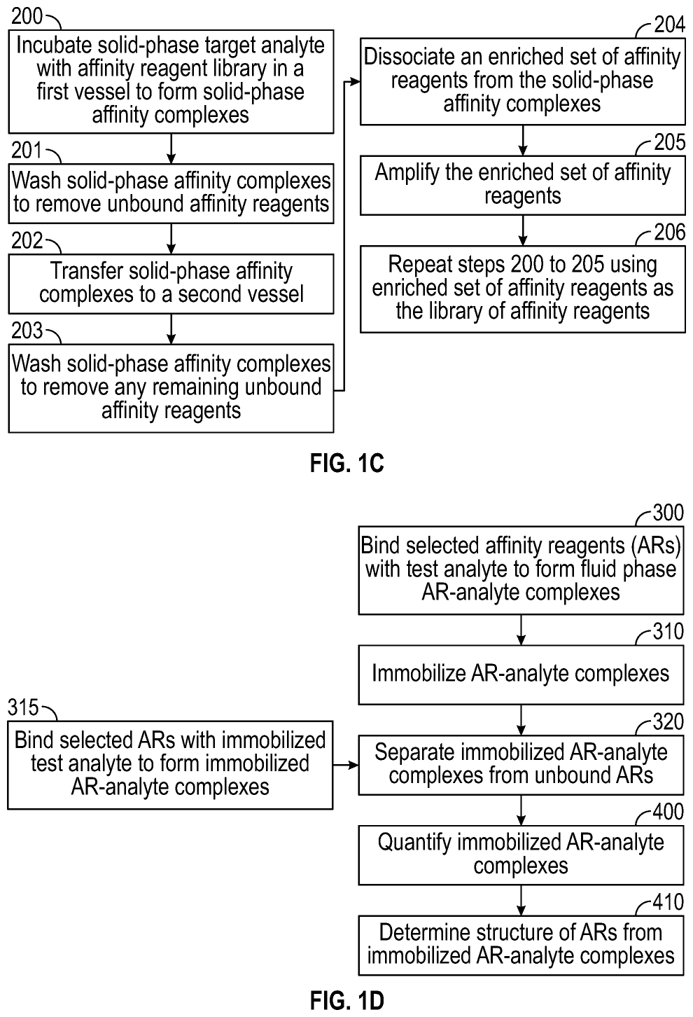

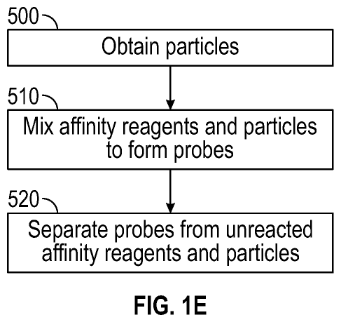

FIG. 1A shows a flow chart describing an exemplary process for preparing affinity reagents. The first step 10 of the process is to obtain an affinity reagent library. In some cases, an appropriate library is available from storage, or a supplier such as a commercial source or collaborating laboratory. In other cases, an appropriate library can be made using methods set forth herein or known in the art, for example, as diagrammed in the flow chart of FIG. 1B or as set forth in further detail herein. In step 20, a selection process is performed on the affinity reagent library to select affinity reagents that bind to target analytes of interest. For example, in configurations that use aptamer-based affinity reagents, a SELEX process can be carried out at this step. Optionally, the selection process can be carried out as set forth in FIG. 1C or as set forth in further detail in the present disclosure. Proceeding to step 30, the binding of selected affinity reagents to test analytes can be assessed. The assessment can optionally be carried out on a bulk pool of selected affinity reagents to determine the level of enrichment of the pool for affinity reagents of interest. An advantage of the binding assessment step is that the results can be used to triage whether to proceed with subjecting the pool of selected affinity reagents to expensive and time-consuming downstream processes for characterizing structure and function of selected affinity reagents. Optionally, assessment of binding for selected affinity reagents and determination of their structure or function can be carried out as set forth in FIG. 1D or as set forth in further detail in the present disclosure. At step 50, probes can be prepared using affinity reagents having desired properties. For example, affinity reagents can be attached to structured nucleic acid particles (SNAPs) as set forth in FIG. 1E.

The steps shown in FIG. 1A are optional as is the order in which they are presented. One or more steps shown in FIG. 1A or otherwise set forth herein can be omitted. Moreover, particular steps can be repeated. In some cases, one or more steps are repeated in a cycle. In one option, step 20 can be repeated after step 30 for a pool of selected affinity reagents, for example, if binding is assessed to be below a given threshold level at step 30. In another option, steps 20 and 30 can be repeated after step 40 for a given pool of selected affinity reagents, for example, if the selected affinity reagents are not found to have desired structural or functional properties. Other cycles can include step 60, which is performed to modify selected affinity reagents to produce an affinity library. A library produced at step 60 can then be reintroduced to the method at step 10 and the subsequent steps repeated. As shown, a cycle can include (a) steps 10, 20 and 60; (b) steps 10, 20, 30 and 60, or (c) steps 10, 20, 30, 40 and 60.

FIG. 1B shows a flow chart describing an exemplary process for obtaining an affinity reagent library. The method is particularly useful for aptamers but can be used for other affinity reagents. A progenitor affinity reagent is obtained at step 100 and modified at step 110 to produce a library of affinity reagents. At step 120, the affinity reagent library can be amplified. For example, an aptamer library can be amplified using PCR or methods set forth herein. Optionally, the method can be used to obtain a structured aptamer library produced by mutagenesis of a progenitor aptamer. The progenitor aptamer can have known or desired binding properties. A structured aptamer library can include variants of a progenitor aptamer having improved binding characteristics compared to the progenitor aptamer. Alternatively or additionally, a structured aptamer library can include variants of a progenitor aptamer that recognize target epitopes that are not substantially recognized by the progenitor aptamer. The libraries are particularly useful inputs to a selection process such as a SELEX process, for obtaining useful variant aptamers. Accordingly, structured aptamer libraries can be useful for evolving aptamers to have improved binding characteristics or different characteristics compared to a progenitor aptamer.

FIG. 1C shows a flowchart describing an exemplary affinity reagent selection process. The process starts at step 200: incubating solid-phase target analyte (e.g. bait) with a library of affinity reagents in a first vessel to form a first set of affinity complexes. In multiplex formats, multiple affinity reagent libraries and/or multiple target analytes (e.g. baits) can be processed in a multiwell plate or other format that supports parallel processing of multiple vessels. At step 201 the solid-phase affinity complexes (e.g. affinity reagent-bait complexes) are washed to remove contaminant affinity reagents and other contaminants. Washing can be carried out in the first vessel or in another vessel to which the solid-phase affinity complexes have been transferred. Washing can be carried out by removing fluid surrounding the solid-phase affinity complexes, delivering a wash fluid to the solid-phase affinity complexes and then removing the wash fluid. Fluids can be added to the vessel using pipette or other fluidic delivery technique. Fluids can be removed by aspiration (e.g. via pipette), decanting, or filtration. Particularly useful vessels have porous filters that retain the solid-phase affinity complexes while passing fluid, thereby separating the fluid from the solid-phase affinity complexes. Filter bottom wells of multiwell plates are useful for multiplex configurations. Washing can be repeated any number of times in a given vessel.

At step 202 the solid-phase affinity complexes can be transferred to a second vessel. For example, the solid-phase affinity complexes can be transferred from a well in a first multi-well plate to a well in a second multi-well plate. Alternatively, the solid-phase affinity complexes may be transferred from one well to another well in the same multi-well plate. Following transfer, step 203 can be carried out to wash the transferred solid-phase affinity complexes in the second vessel. The wash step 203 can be performed as set forth above for step 201 and can optionally be repeated one or more times. Moreover, the transfer (202) and wash (203) steps can be repeated one or more times if desired. The method can then move to step 204 where an enriched set of affinity reagents is dissociated from the solid-phase affinity complexes. The enriched set of affinity reagents can optionally be amplified to produce affinity reagent amplicons. For example, nucleic acid aptamers and other nucleic acid-based affinity reagents can be amplified using techniques such as preparative polymerase chain reaction (PCR) or analytical PCR (e.g. quantitative PCR or real time PCR). At step 205, the enriched set of affinity reagents, or amplicons thereof, can optionally be subjected to further selection by repeating steps 200 to 205. Alternatively, one or more affinity reagents from the enriched set can be used in a binding reaction or other application.

Different vessels may differ with respect to the chemical composition of vessel walls, surface treatments present on the surface, presence or absence of contaminants, shape of the vessels, surface area of vessel walls, porosity of the vessel walls, presence or absence of filters in vessels, porosity of the filters or the like. Whether or not the vessels differ in one or more of these ways, the vessels may differ due to having been treated differently, for example, prior to, or during, use in one or more step of a method set forth herein. For example, two vessels may have been washed with different solutions; one vessel may have been washed whereas the other was not; two vessels may have been stored under different conditions such as different temperature, intensity of light, spectrum of light or humidity, or one vessel may have been used in a prior selection process whereas the other has not. A substantial improvement in selection of affinity reagents is provided by the methods set forth herein, not merely due to washing solid-phase target analytes but also due to transfer of the solid-phase target analytes to a different vessel (e.g. different wells or well plates). Typically, the different vessel is uncontaminated by any affinity reagents, for example, due to not having been contacted with affinity reagents that are being subjected to the selection process. The process of transferring solid-phase analytes to different vessels and washing in the vessels may be repeated further until undesirable affinity reagent contamination is minimized to a useful level. It will be understood that two or more vessels used in a method, composition or apparatus of the present disclosure need not differ substantially with respect to one or more characteristic set forth herein. Moreover, the two or more vessels may be substantially the same in some configurations of the methods, compositions or apparatus set forth herein.

The present disclosure provides improved enrichment of affinity reagents. Vessels, such as multiwell plates, used for manipulation of affinity reagents and other useful molecules have high surface areas where contaminant affinity reagents may in some cases be non-specifically retained. Contaminant affinity reagents if carried over with a desired fraction of affinity reagents can pose a substantial contamination risk. The success of selection processes, such as SELEX processes, can be improved by minimizing contamination by unwanted affinity reagents. For example, unwanted aptamer affinity reagent contaminants which are carried over to a PCR may adversely impact amplification of desired aptamers due to competition for PCR reagents, in some cases even to the point of substantially excluding amplification of desired aptamers.

Particular aspects of the methods, compositions and apparatus set forth herein can provide valuable improvements to bait molecules used to display target epitopes during processes for selecting affinity reagents that bind the epitopes, such as SELEX procedures used for selecting aptamers. Selection schemes provide a powerful tool in the molecular biology arsenal for acquiring affinity reagents that are specific to a variety of targets. A library containing a plurality of different candidate affinity reagents can be contacted with bait molecules having a target epitope and those candidate affinity reagents that bind to the bait molecule can be separated from those in the mixture that do not. In many cases, the bait molecule will include not only the target epitope but other moieties, for example, linker moieties that attach the target epitope to an object that can be separated from the screening mixture. Unfortunately, this can result in the selection of candidate affinity reagents that recognize or bind these other moieties. For example, the selected candidate affinity reagents may recognize or bind the other moieties instead of the target epitope, or they may require presence of the other moieties to recognize or bind the target epitope. Selection of affinity reagents against short peptide epitopes (e.g. sequences of 2, 3, 4, 5, or 6 amino acids) can be particularly challenging since the bait peptide often includes flanking moieties of one or more amino acids on one or both sides of the peptide epitope. The problem of selecting affinity reagents that are specific for short peptide epitopes independent of their sequence context can be solved using bait polypeptides having custom flanks that improve presentation of the peptide epitopes for binding to affinity reagents.

The present disclosure provides a polypeptide having a peptide epitope flanked on one or both sides by a flanking moiety, wherein the flanking moiety includes one or more amino acids. One or both flanking moieties can be configured to have a three-dimensional structure that orients the peptide epitope as a moiety that is spatially separated from the rest of the polypeptide. One or both flanking moieties can present the polypeptide epitope to bind an affinity reagent such that the affinity reagent need not interact with the flanking region(s) or such that the flanking region(s) do(es) not contribute substantially to the affinity of the epitope for the affinity reagent. Accordingly, the affinity reagent need not recognize any moiety of the polypeptide other than the peptide epitope.

A particularly useful polypeptide includes a trimeric peptide epitope embedded in a polypeptide sequence that forms a corner motif that is found in DNA binding proteins. This structural motif has been identified from a comparison of three-dimensional structures for DNA binding proteins and has been shown to result from a relatively wide variety of different amino acid sequences (Wu et al. Nucl. Acids Res. 38:14 (2010), which is incorporated herein by reference). The present disclosure provides polypeptides having primary sequences that form corner motifs or other similar structures for presentation of peptide epitopes. The polypeptides are shown to be particularly useful for use as bait molecules when selecting aptamers for binding to trimeric peptide epitopes. Moreover, the corner motif can be used with peptide epitopes other than trimers. For example, epitopes having a sequence of more than three amino acids can be used. Larger sequences, for example, those having multiple peptide epitopes, can be embedded in a polypeptide sequence that forms a corner motif. The polypeptides set forth herein can also be useful for selection of affinity reagents other than aptamers such as antibodies (e.g. full length antibodies or functional fragments thereof).

FIG. 1D shows a flow chart describing an exemplary process for evaluating binding of affinity reagents to test analytes and characterizing structures of the affinity reagents. The process can begin at step 300, wherein a binding assay is performed between selected affinity reagents and test analytes in fluid phase. Complexes that form between the affinity reagent and test analyte can be immobilized at step 310 to allow subsequent separation of bound affinity reagents from unbound affinity reagents at step 320. In an alternative option, the process can start at step 315, wherein a binding assay is performed between selected affinity reagents in fluid-phase and test analytes immobilized on a solid-phase support. The bound affinity reagents can be separated from unbound affinity reagents at step 320. The fluid-phase binding option (starting at step 300) can provide an advantage of being tailored for selection of affinity reagents having high affinity for target analytes in fluid-phase. This option also reduces unwanted selection of affinity reagents that have affinity for solid-phase supports used in steps 315 and 320.

In either option shown in FIG. 1D, a pool of many different affinity reagents, having been obtained from a selection process, can be contacted with test analytes, thereby allowing a bulk assessment of the pool. For example, the pool can be quantified to determine the number of selected affinity reagents in the pool or the fraction of affinity reagents that have been selected relative to the number of affinity reagents in the library that was subjected to the selection process. The results can be evaluated as a triage to determine the fate for the pool of selected affinity reagents. For example, the pool can proceed to step 410 where the affinity reagents are evaluated for structure, function or both. In many cases, evaluation of structure or function for the affinity reagents is expensive and time consuming. For example, a pool of aptamers can be subjected to next generation sequencing which requires expensive instrumentation, reagents and computation. An advantage of performing a binding assessment process, such as the one shown in FIG. 1D, is that an affinity reagent pool that does not achieve a quality threshold can be subjected to further rounds of modification and/or selection, or discarded. Moreover, the results of a binding assessment process can be used to evaluate the selection process itself. For example, the steps or reagents used in the process can be modified and then the quality or quantity of the affinity reagents produced by the modified process can be evaluated to determine desirability of the modification.

FIG. 1E shows a flow chart describing an exemplary process for preparing probes having affinity reagents as components. The process can begin at step 500 by obtaining particles. In some configurations of the present methods, the particles are structured nucleic acid particles. Structured nucleic acid particles can be made using methods set forth herein. At step 510 affinity reagents are attached to particles. Step 510 can be carried out during or after step 500. For example, structured nucleic acid particles can be formed prior to being attached to affinity reagents. Alternatively, affinity reagents can be present during formation of structured nucleic acid particles such that the affinity reagents form integral components of the particles. At step 520, probes can be separated from affinity reagents and particles that did not participate in formation of the probes.

### Definitions

Terms used herein will be understood to take on their ordinary meaning in the relevant art unless specified otherwise. Several terms used herein and their meanings are set forth below.

As used herein, the term “affinity reagent” refers to a molecule or other substance that is capable of specifically or reproducibly binding to an analyte (e.g. protein or peptide). Optionally, an affinity reagent can selectively bind (or recognize) one or more particular epitope(s) in a sample without having substantial affinity for any other epitopes known or suspected of being in the sample. An affinity reagent can be larger than, smaller than or the same size as the analyte. An affinity reagent may form a reversible or irreversible bond with an analyte. An affinity reagent may bind with an analyte in a covalent or non-covalent manner. Affinity reagents may include reactive affinity reagents, catalytic affinity reagents (e.g., kinases, proteases, etc.) or non-reactive affinity reagents (e.g., antibodies or fragments thereof). An affinity reagent can be non-reactive and non-catalytic, thereby not permanently altering the chemical structure of an analyte to which it binds. Nucleic acid-based affinity reagents, such as nucleic acid aptamers are particularly useful affinity reagents. Affinity reagents that are not nucleic acid-based or that do not contain nucleic acids can be useful. For example, an affinity reagent can be an antibody, such as a full-length antibody or functional fragment thereof. The term “antibody” is used herein to refer to any epitope-binding molecule or molecular complex having at least one complementarity determining region (CDR) that binds to or interacts with a particular epitope with high affinity. A functional fragment of an antibody can include any fragment that is capable of binding to an epitope with a detectable affinity, such as a Fab, Fab′, F(ab′)₂, single-chain variable (scFv), di-scFv, tri-scFv, or microantibody. Other useful affinity reagents include, for example, affibodies, affilins, affimers, affitins, alphabodies, anticalins, avimers, DARPins, monobodies, nanoCLAMPs, protein aptamers, lectins or functional fragments thereof.

As used herein, the term “amplicon” refers to the product of copying a nucleic acid, wherein the product has a nucleotide sequence that is the same as or complementary to at least a portion of the nucleotide sequence of the nucleic acid. An amplicon can be produced by any of a variety of amplification methods that use the nucleic acid, or an amplicon thereof, as a template including, for example, polymerase extension, polymerase chain reaction (PCR), rolling circle amplification (RCA), ligation extension, or ligation chain reaction. A first amplicon of a target nucleic acid is typically a complementary copy. Subsequent amplicons can be copies that are created from the target nucleic acid or from an amplicon thereof. A subsequent amplicon can have a sequence that is substantially complementary to the target nucleic acid or substantially identical to the target nucleic acid.

As used herein, the term “aptamer,” refers to a nucleic acid or peptide that specifically or reproducibly binds a particular epitope. Optionally, an aptamer can selectively bind (or recognize) one or more particular epitope(s) in a sample without having substantial affinity for any other epitopes known or suspected of being in the sample. A nucleic acid aptamer can be composed of DNA, RNA or analogs thereof. An aptamer can bind an epitope through various non-covalent interactions such as, electrostatic interactions, hydrophobic interactions, hydrogen bonding interactions, van der Waals interactions, and induced fit.

As used herein, the term “attached” refers to the state of two things being joined, fastened, adhered, connected or bound to each other. For example, an analyte, such as a protein, can be attached to a solid-phase component by a covalent or non-covalent bond. A covalent bond is characterized by the sharing of pairs of electrons between atoms. A non-covalent bond is a chemical bond that does not involve the sharing of pairs of electrons and can include, for example, hydrogen bonds, ionic bonds, van der Waals forces, hydrophilic interactions and hydrophobic interactions.

As used herein, the term “click chemistry” refers to single-step, thermodynamically-favorable conjugation reaction utilizing biocompatible reagents. A click reaction may utilize no toxic or biologically incompatible reagents (e.g., acids, bases, heavy metals) or generate no toxic or biologically incompatible byproducts. A click reaction may utilize an aqueous solvent or buffer (e.g., phosphate buffer solution, Tris buffer, saline buffer, MOPS, etc.). A click reaction may be thermodynamically favorable if it has a negative Gibbs free energy of reaction, for example a Gibbs free energy of reaction of less than about −5 kiloJoules/mole (kJ/mol), −10 kJ/mol, −25 kJ/mol, −50 kJ/mol, −100 kJ/mol, −200 kJ/mol, −300 kJ/mol, −400 kJ/mol, or less than −500 kJ/mol. Exemplary bioorthogonal and click reactions are described in detail in WO 2019/195633A1, which is herein incorporated by reference. Exemplary click reactions may include metal-catalyzed azide-alkyne cycloaddition, strain-promoted azide-alkyne cycloaddition, strain-promoted azide-nitrone cycloaddition, strained alkene reactions, thiol-ene reaction, Diels-Alder reaction, inverse electron demand Diels-Alder reaction, [3+2] cycloaddition, [4+1] cycloaddition, nucleophilic substitution, dihydroxylation, thiol-yne reaction, photoclick, nitrone dipole cycloaddition, norbornene cycloaddition, oxanobornadiene cycloaddition, tetrazine ligation, and tetrazole photoclick reactions. Exemplary functional groups or reactive handles utilized to perform click reactions may include alkenes, alkynes, azides, epoxides, amines, thiols, nitrones, isonitriles, isocyanides, aziridines, activated esters, and tetrazines. Other well-known click conjugation reactions may be used having complementary bioorthogonal reaction species, for example, where a first click component comprises a hydrazine moiety and a second click component comprises an aldehyde or ketone group, and where the product of such a reaction comprises a hydrazone functional group or equivalent.

As used herein, the term “comprising” is intended herein to be open-ended, including not only the recited elements, but further encompassing any additional elements.

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, the term “epitope” refers to an affinity target within a protein, peptide or other analyte. Epitopes may comprise amino acid sequences that are sequentially adjacent in the primary structure of a protein (i.e. a linear epitope) or amino acids that are structurally adjacent in the secondary, tertiary or quaternary structure of a protein (i.e. conformational epitopes). An epitope can optionally be recognized by or bound to an antibody. However, an epitope need not necessarily be recognized by any antibody, for example, instead being recognized by an aptamer or other affinity reagent. An epitope can optionally bind an antibody to elicit an immune response. However, an epitope need not necessarily participate in, nor be capable of, eliciting an immune response.

As used herein, the term “exogenous,” when used in reference to a moiety of a molecule, means a moiety that is not present in a natural analog of the molecule. For example, an exogenous moiety of a nucleic acid is a moiety that is not present on a naturally occurring nucleic acid. Similarly, an exogenous label that is present on a binding reagent is a label that is not found on the binding reagent in its native milieu.

As used herein, the term “fluid-phase,” when used in reference to a molecule, means the molecule is in a state wherein it is mobile in a fluid, for example, being capable of passively diffusing through the fluid. A fluid-phase molecule is not in a state of immobilization on a solid-phase support nor attachment to a solid-phase support.

As used herein, the term “isolated” means a substance in a form or environment that does not occur in nature. Non-limiting examples of isolated substances include (1) any non-naturally occurring substance, (2) any substance that is at least partially removed from one or more or all of the naturally occurring constituents with which it is associated in nature; or (3) any substance modified by human intervention relative to that substance found in nature. An isolated substance may be, for example, an affinity reagent, epitope, binding partner (e.g. for an affinity reagent), nucleic acid, or polypeptide (e.g. protein or peptide). A substance that is in vitro is referred to herein as being isolated independent of the substance's source or composition. As used herein, the term, “multiwell plate” means a solid-phase support comprising a plurality of concave sample receptacles. A well plate can have a standard or non-standard size. For example, a common standard is a 96 well format. Other well plate formats may include, 1, 3, 4, 6, 12, 24, 48, 384 and 1536 or higher, and may include well plates comprising any number of wells between 1 to 1536 inclusive or higher, where the numerical values, 1 to 1536 or higher are integers. The well sizes are usually uniform in a given well plate, but can vary in some configurations. Well volume may be, for example, at least 1 nL, 10 nL, 100 nL, 1 uL, 10 uL, 100 uL, 1 mL, 10 mL or more. Alternatively or additionally, well volume can be at most 10 mL, 1 mL, 100 uL, 10 uL, 1 uL, 100 nL, 10 nL, 1 nL or lower. Wells may be round/cylindrical, flat-bottom, square, U-shaped bottom, or any other shape or bottom type. Wells may contain porous filters, for example, positioned at the bottom of each well, through which a fluid can be expelled from the well. The porous filter can optionally have a pore size that retains beads or particles. Accordingly, filters can be used for partitioning solid and liquid phases, for example, for separating solid-phase supports from a liquid suspension or for washing a solid-phase support with a washing solution.

As used herein, the term “nucleic acid origami” refers to a nucleic acid construct having an engineered tertiary or quaternary structure. A nucleic acid origami may include DNA, RNA, PNA, modified nucleotides, non-natural nucleotides, or combinations thereof. A nucleic acid origami may include a plurality of oligonucleotides that hybridize via sequence complementarity to produce the engineered structure of the origami. A nucleic acid origami may include sections of single-stranded or double-stranded nucleic acid, or combinations thereof. Exemplary nucleic acid origami structures may include nanotubes, nanowires, cages, tiles, nanospheres, blocks, or combinations thereof. A nucleic acid origami can optionally include a relatively long scaffold nucleic acid to which multiple smaller nucleic acids hybridize, thereby creating folds and bends in the scaffold that produce an engineered structure. The scaffold nucleic acid can be circular or linear. The scaffold nucleic acid can be single stranded but for hybridization to the smaller nucleic acids. A smaller nucleic acid (sometimes referred to as a “staple”) can hybridize to two regions of the scaffold, wherein the two regions of the scaffold are separated by an intervening region that does not hybridize to the smaller nucleic acid. In some configurations, a staple need not hybridize to more than one region of a scaffold or need not hybridize to a scaffold at all, for example, hybridizing to another staple instead. Moreover, a staple can include one or more regions that are not hybridized to any other portion of a nucleic acid origami.

As used herein, the term “particle” or “bead” means a solid-phase support having a largest dimension between 500 nm and 1 mm. The solid-phase support can be composed of a rigid or semi-rigid material. The particle or bead can be insoluble in a fluid such as aqueous liquid. A particle or bead can have a shape characterized, for example, as a sphere, ovoid, polyhedron, or other recognized particle shape whether having regular or irregular dimensions. A particle or bead can be made of biological (e.g. nucleic acid such as nucleic acid origami) or non-biological materials (e.g. agarose, glass, silica or quartz). Structured nucleic acid particles can be useful, such as those set forth in U.S. Pat. No. 11,203,612, US Pat. App. Pub. No. 2022/0162684 A1; or U.S. patent application Ser. No. 17/692,035, each of which is incorporated herein by reference. Magnetic or paramagnetic beads can be particularly useful due to the ease of manipulation using magnets at various steps of the methods described herein.

As used herein, the term “polypeptide” refers to a molecule comprising two or more amino acids joined by a peptide bond. A polypeptide may also be referred to as a protein, oligopeptide or peptide. Although the terms “protein,” “polypeptide,” “oligopeptide” and “peptide” may optionally be used to linguistically distinguish molecules having different characteristics, such as amino acid sequence composition or length, molecular weight, origin of the molecule or the like, the terms are not intended to inherently include such distinctions in all contexts. A polypeptide can be a naturally occurring molecule, or synthetic molecule. A polypeptide may include one or more non-natural amino acids, modified amino acids, or non-amino acid linkers. A polypeptide may contain D-amino acid enantiomers, L-amino acid enantiomers or both. Amino acids of a polypeptide may be modified naturally or synthetically, such as by post-translational modifications.

As used herein, the term “solid-phase” or “immobilized,” when used in reference to a molecule, refers to the molecule being associated with a solid-phase support. For example, the molecule can be confined at, or attached to, a solid-phase material. Immobilization can be temporary (e.g. for the duration of one or more steps or methods set forth herein) or permanent. A solid-phase molecule can be in contact with a fluid-phase and prevented from diffusing in the fluid phase. The association between a molecule and solid-phase support can be reversible or irreversible under conditions utilized for a method, system or composition set forth herein.

As used herein, the term “solid-phase support” refers to a substrate that is insoluble in a fluid such as an aqueous liquid. Optionally, the substrate can be rigid. The substrate can be non-porous or porous. The substrate can optionally be capable of taking up a liquid (e.g. due to porosity) but will typically, but not necessarily, be sufficiently rigid that the substrate does not swell substantially when taking up the liquid and does not contract substantially when the liquid is removed. A nonporous solid-phase support is generally impermeable to liquids or gases. Exemplary solid-phase supports include, but are not limited to, glass and modified or functionalized glass, plastics (including acrylics, polystyrene and copolymers of styrene and other materials, polypropylene, polyethylene, polyamide, polyester, polycarbonate, polybutylene, polyurethanes, Teflon™, cyclic olefins, polyimides, polytetrafluoroethylene (PTFE), polyethylene terephthalate (PET) etc.), nylon, ceramics, resins, Zeonor™, silica or silica-based materials including silicon and modified silicon, carbon, metals, inorganic glasses, optical fiber bundles, gels, and polymers.

As used herein, the term “structured nucleic acid particle” or “SNAP” refers to a single- or multi-chain polynucleotide molecule having a compacted three-dimensional structure. The compacted three-dimensional structure can optionally be characterized in terms of hydrodynamic radius or Stoke's radius of the SNAP relative to a random coil or other non-structured state for a nucleic acid having the same mass or polynucleotide length as the SNAP. The compacted three-dimensional structure can optionally be characterized with regard to tertiary structure. For example, a SNAP can be configured to have an increased number of internal binding interactions between regions of a polynucleotide strand, less distance between the regions, increased number of bends in the strand, and/or more acute bends in the strand, as compared to a nucleic acid molecule of similar length in a random coil or other non-structured state. Alternatively or additionally, the compacted three-dimensional structure can optionally be characterized with regard to tertiary or quaternary structure. For example, a SNAP can be configured to have an increased number of interactions between polynucleotide strands or less distance between the strands, as compared to a nucleic acid molecule of similar length in a random coil or other non-structured state. In some configurations, the secondary structure of a SNAP can be configured to be more dense than a nucleic acid molecule of similar length in a random coil or other non-structured state. A SNAP may contain DNA, RNA, PNA, modified or non-natural nucleic acids, or combinations thereof. A SNAP may include a plurality of oligonucleotides that hybridize to form the SNAP structure. The plurality of oligonucleotides in a SNAP may include oligonucleotides that are attached to other molecules (e.g., probes, analytes such as proteins, reactive moieties, or detectable labels) or are configured to be attached to other molecules (e.g., by functional groups). A SNAP may include engineered or rationally designed structures. Exemplary SNAPs include nucleic acid origami and nucleic acid nanoballs.

As used herein, the term “vessel” refers to an enclosure that contains a substance. The enclosure can be permanent or temporary with respect to the timeframe of a method set forth herein or with respect to one or more steps of a method set forth herein. Exemplary vessels include, but are not limited to, a well (e.g. in a multiwell plate or array of wells), test tube, channel, tubing, pipe, flow cell, bottle, vesicle, droplet that is immiscible in a surrounding fluid, or the like. A vessel can be entirely sealed to prevent fluid communication from inside to outside, and vice versa. Alternatively, a vessel can include one or more ingress or egress to allow fluid communication between the inside and outside of the vessel. A vessel can be made from multiple materials, for example, including a well having a non-porous material and a mesh filter.

The embodiments set forth below and recited in the claims can be understood in view of the above definitions.

### Selection of Affinity Reagents

The present disclosure provides a method of selecting at least one affinity reagent. The method can include steps of (a) contacting a solid-phase analyte with a fluid-phase library of different affinity reagents in a vessel, wherein the solid-phase includes a particle to which the analyte is attached, wherein the vessel includes a porous filter that prevents passage of the particle; (b) passing the fluid-phase library of affinity reagents through the porous filter to separate affinity reagents of the library from the solid-phase analyte; (c) transferring the solid-phase analyte from the vessel to a second vessel; and (d) washing the solid-phase analyte in the second vessel to remove further affinity reagents of the library from the solid-phase analyte, whereby at least one of the different affinity reagents remains bound to the solid-phase analyte.

A method of the present disclosure can be used to select an affinity reagent that is capable of specifically or reproducibly binding to an analyte. For ease of explanation, methods are exemplified herein in the context of using aptamers as affinity reagents. However, any of a variety of affinity reagents can be employed in a similar way as appropriate to the composition and characteristics of the affinity reagent. For example, protein-based affinity reagents can be affibodies, affilins, affimers, affitins, alphabodies, anticalins, avimers, miniproteins, designed ankyrin repeat proteins (DARPins), peptide aptamers, peptoid aptamers, antibodies, monobodies, nanoCLAMPs, or lectins. Functional fragments of such affinity reagents can be used such as fragments of antibodies that specifically or reproducibly bind to analytes, examples of which include, Fab′ fragments, F(ab′)₂ fragments, single-chain variable fragments (scFv), di-scFv, tri-scFv, or microantibodies.

In particular configurations of the compositions, apparatus or methods set forth herein, the affinity reagents are aptamers. For example, a library of different aptamers can be employed and one or more of the different aptamers can be selected using a method set forth herein. Nucleic acid aptamers can be particularly useful, for example, because they can be conveniently replicated and amplified. A nucleic acid aptamer can have a primary sequence length that is relatively short for example being shorter than 100, 75, 60, 50, 40, 30, 25 or fewer nucleotides in length. Alternatively or additionally, a nucleic acid aptamer can have a length of at least 25, 30, 40, 50, 60, 75, 100 or more nucleotides. A nucleic acid aptamer library can include members in one or both of the above size ranges. As set forth in further detail below, an aptamer library can be based on a nucleic acid construct having a variable sequence region flanked by priming site regions. The variable sequence region, which typically includes nucleotides that are involved in binding to an analyte of interest, can have a length in one or both of the size ranges set forth above.

Nucleic acid aptamers may include natural nucleotides. For example, DNA aptamers may include deoxycytidine, deoxyguanine, deoxythymine and/or deoxyadenine. RNA aptamers can include uracil, guanine, cytidine or adenine. In some cases, nucleic acid aptamers may include one or more non-natural nucleotide analog. Aptamers may include exogenous moieties at any suitable position on the nucleotide base, sugar or phosphate groups. Chemical modifications can provide the benefit of a greater range of chemical diversity than aptamers comprising naturally occurring bases, sugars and phosphate groups. For example, aptamers may include 2′-fluoro nucleotide ribose sugars. Alternatively or additionally, aptamers may include unnatural bases, for example, derived from 5-ethynyl uridine (5-EdU) click chemistry. The latter is particularly suited to aptamer generation as the 5-EdU can effectively participate in PCR amplification steps. Aptamers with 5-EdU groups may be amplified and then coupled with a wide range of chemical groups comprising an azide group via well-known click chemistry methodologies. For example, aptamers comprising 5-EdU groups may be coupled with a C-12 alkane azide or an azido steroid to afford aptamers with large hydrophobic components.

Nucleic acid aptamers are particularly useful since they can be copied or amplified using available techniques such as polymerase chain reaction (PCR). Accordingly, a method set forth herein can include a step of amplifying one or more different aptamers to produce a plurality of different aptamer amplicons.

By way of example, a method of selecting at least one affinity reagent, such as an aptamer or other nucleic acid based-affinity reagent, can include steps of (a) contacting a solid-phase analyte with a fluid-phase library of different affinity reagents in a vessel, wherein the solid-phase includes a particle to which the analyte (e.g. bait molecule) is attached, wherein the vessel includes a porous filter that prevents passage of the particle; (b) passing the fluid-phase library of affinity reagents through the porous filter to separate contaminant affinity reagents of the library from the solid-phase, thereby retaining the solid-phase analyte and affinity reagents; (c) transferring the retained solid-phase from the vessel to a second vessel; (d) washing the solid-phase analyte in the second vessel to optionally remove retained affinity reagents of the library from the solid-phase analyte, whereby at least one of the different affinity reagents remains bound to the solid-phase analyte (e) dissociating at least one of the different affinity reagents from the solid-phase analyte of step (e); and (f) amplifying the at least one dissociated affinity reagent to produce a plurality of different affinity reagent amplicons. Optionally, the method can further include (g) repeating steps (a)-(d) using the plurality of different affinity reagent amplicons instead of the library, thereby selecting at least one affinity reagent amplicon that is bound to the solid-phase analyte.

PCR can be used as a preparative tool to produce multiple copies of an aptamer or other nucleic acid. PCR can also be configured to select for particular aptamers, or other types of nucleic acids, based on the presence of particular structural features such as priming sites that complement particular amplification primers or intact template regions that can be copied by polymerase extension of amplification primers. Real-time qPCR provides a robust analytical tool for quantifying and characterizing nucleic acids and can be used for aptamers having appropriate structural features to accommodate amplification (e.g. priming sites that can hybridize to amplification primers).

PCR can be used to amplify an aptamer (or other nucleic acid-based affinity reagent) which has been recovered from a binding complex formed with a target analyte such as a target polypeptide. An aptamer is typically composed of a single strand of nucleic acid (e.g. a single strand of DNA or RNA) but can be copied by a polymerase to form double-stranded DNA for PCR amplification or other purposes. A reverse transcriptase can be used to convert an RNA-based affinity reagent to DNA (e.g. double stranded DNA or single stranded DNA) for PCR.

A library of aptamers (or other nucleic acid-based affinity reagents) can be structured to have a variable region flanked by universal priming sites. Individual aptamers in the library can differ from each other with regard to the sequence of nucleotides in their variable regions. The sequence differences can occur in the binding sites of the aptamers (e.g. the region of the aptamer that interacts with a bound analyte) or in regions other than the binding sites that nonetheless may influence binding activity or other characteristics of the aptamer (e.g. stability of the aptamer, solubility of the aptamer etc.). The use of universal binding sites can allow for convenient and unbiased amplification of the members of an aptamer library using a small set of universal primers. In accordance with the present methods, a subset of one or more members of an aptamer library can be selected based on binding activity and separated from aptamers lacking the desired binding activity. The selected members can be separated from inactive aptamers and then amplified using universal primers. However, inactive aptamers that fail to be removed during the selection, or that otherwise contaminate a subset of selected aptamers, may be amplified due to the presence of universal binding sites. In many cases, the contaminant aptamers will be present in lower quantities than the selected aptamers. Since PCR is a non-linear process, contaminants that are present in relatively low numbers will amplify at a substantially slower rate than the more predominant aptamers (e.g. selected aptamers) during the early cycles of the PCR reaction. During later cycles, for example as amplification begins to saturate, the amplified contaminants may begin to increase in abundance relative to the quantity of amplicons of the selected aptamers. To minimize the undesirable amplification of contaminants, PCR reactions can be monitored and stopped prior to saturation of the amplification reaction. For example, a PCR reaction can be stopped during the exponential (i.e. log-linear) stage of amplification. A PCR reaction can be stopped at a point where amplification has reached less than 90% saturation, 80% saturation, 70% saturation, 60% saturation, 50% saturation or lower. The use of qPCR for amplification of aptamers, or as a companion assay for monitoring a PCR amplification, can be advantageous for determining when to stop PCR amplification in a method set forth herein.

An affinity reagent or library of affinity reagents can be generated by any of a variety of methods. Such methods can include a process for introducing variability into a core construct for example by random mutagenesis of the core construct, random modification of the core construct, error prone synthesis of the core construct, error prone replication of the core construct or the like. For example, an aptamer or aptamer library can be generated by systematic evolution of ligands by exponential enrichment (SELEX). Particular configurations of the methods, apparatus and compositions of the present disclosure can be used to improve known SELEX procedures such as those set forth in U.S. Pat. Nos. 5,567,588, 5,580,737, 5,683,867, 5,763,595, or 6,001,577; US Pat. App. Pub. No. 2002/6387620 A1, 2002/6376474 A1, 2007/7312325 A1, or 2001/6261774 A1; WO 96/04403; WO 98/33941; WO 00/56930; Gold, J. Mol. Evol, 81:140-143 (2015); Wang et al. Biotech. Adv., 37:28-50 (2019); Gold et al., PloS ONE, 5:12, e15004 (2010); each of which is hereby incorporated by reference. Other methods that can be employed for creation of an affinity reagent or library of affinity reagents include, without limitation, phage display, yeast display, mammalian cell display, insect cell display, ribosome display, particle display, peptimer evolution, peptimer design, and inoculation. Affinity reagents may be designed using structure-based design methods. Such design methods can be deployed in a rational design format in which targeted changes are made to a core construct. Alternatively, a library of affinity reagents can be produced by a semi-rational method in which structure-based design methods are combined with semi-random modification of a core construct. For example, structure-based design methods can be used to choose nucleotide positions of a core construct that are to be modified in a random fashion, and/or to choose the variety of nucleotides or nucleotide analogs to be incorporated at one or more position in the core construct.

Aptamers may include short synthetic single-stranded (ss) nucleic acids, such as those set forth in Tuerk and Gold Science, 1990, Vol 249, pp. 505-510 or Ellington, and Szostak, Nature 1990, 346, 818-822. Aptamers can bind to a broad range of target analytes including, but not limited to, polypeptides, nucleic acids, post-translational modifications of polypeptides (e.g. phosphate moieties, lipid moieties, glycosyl moieties etc.), small molecules such as candidate therapeutics, metal ions, cells, tissues, vitamins, hormones, or metabolites. As a class of affinity reagents, aptamers display some advantages over traditional antibodies such as prolonged shelf life, low batch to batch variation, low levels of immunogenicity, and the flexibility to incorporate chemical modifications for enhanced stability and targeting affinity.

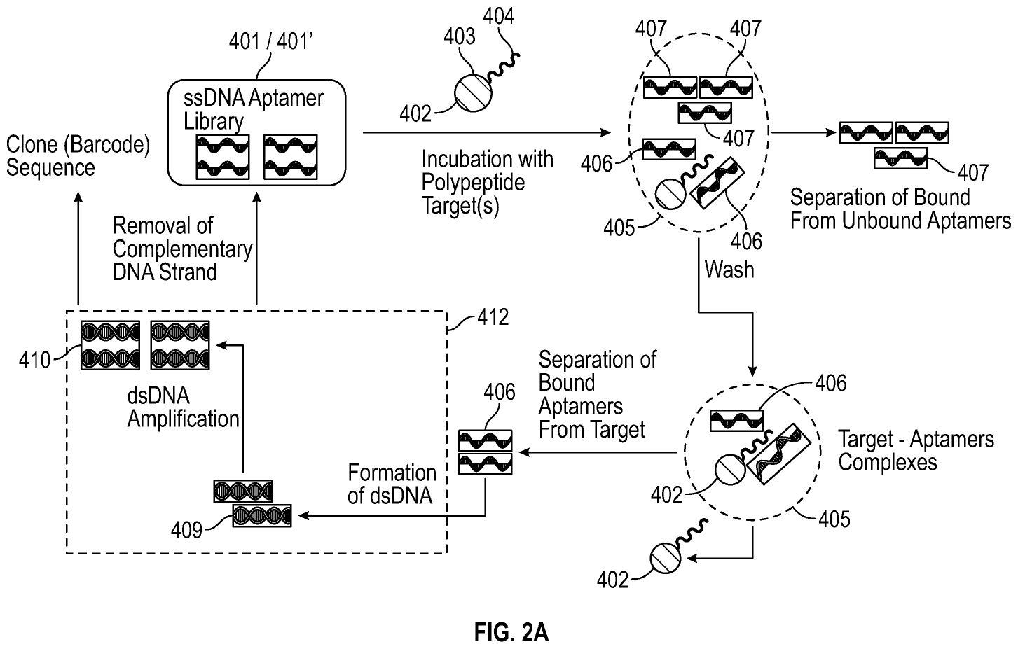

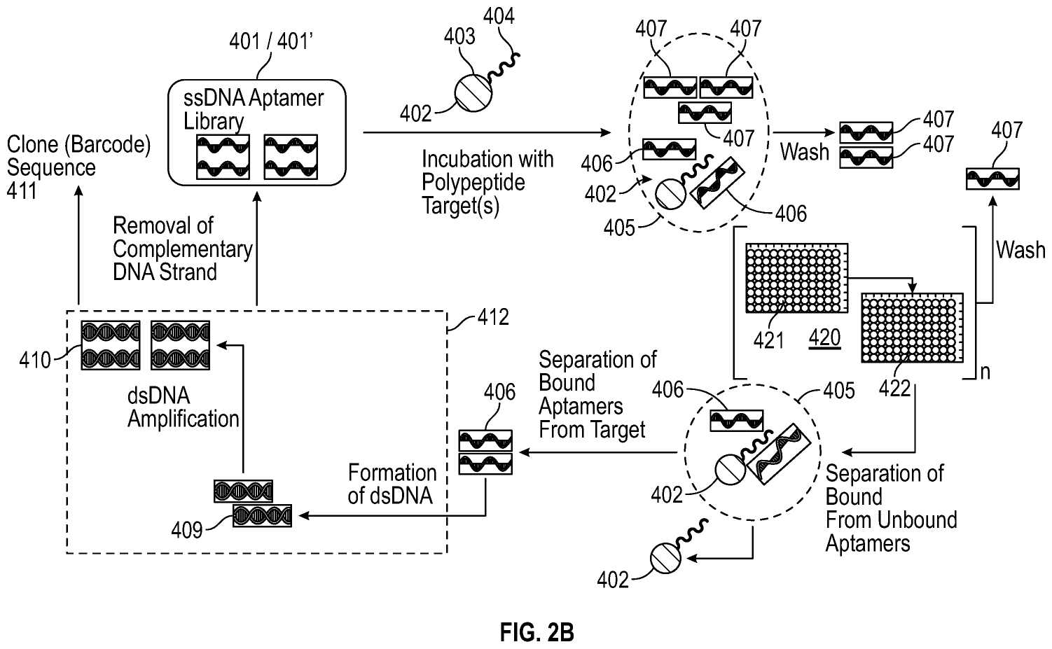

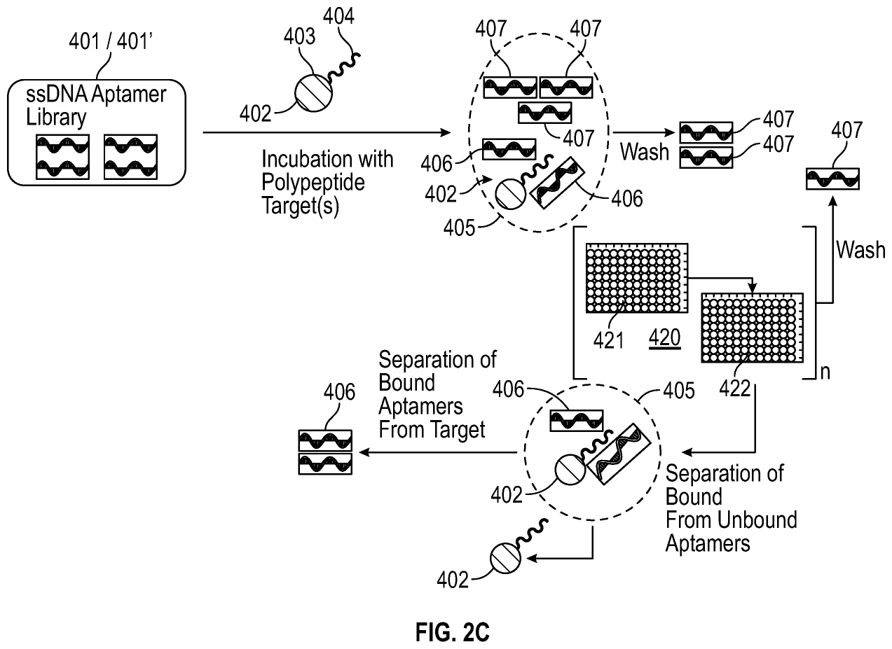

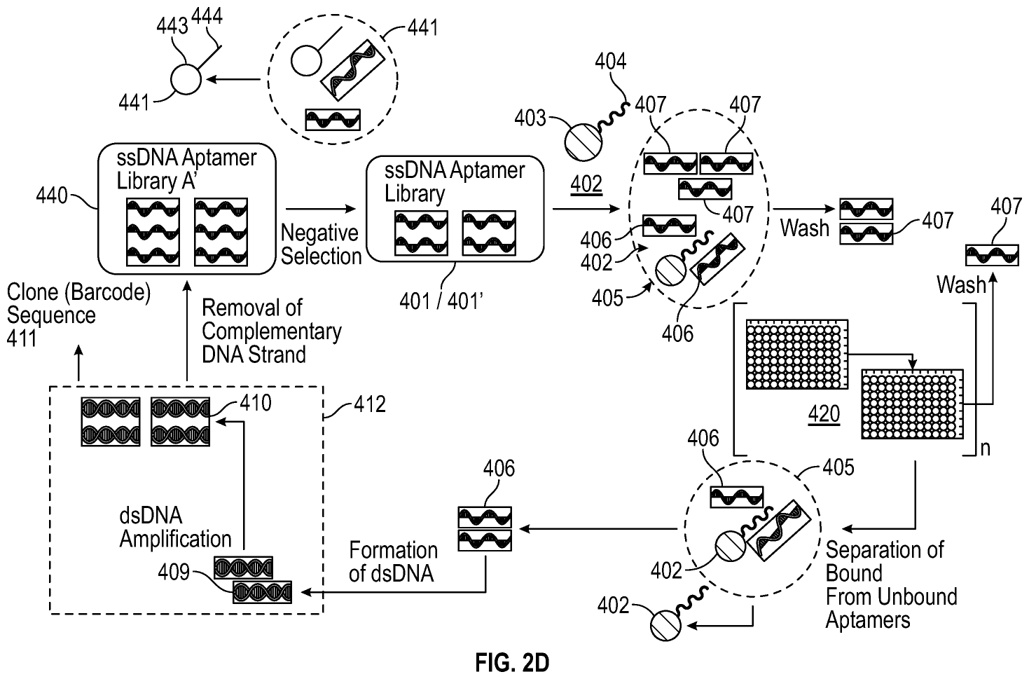

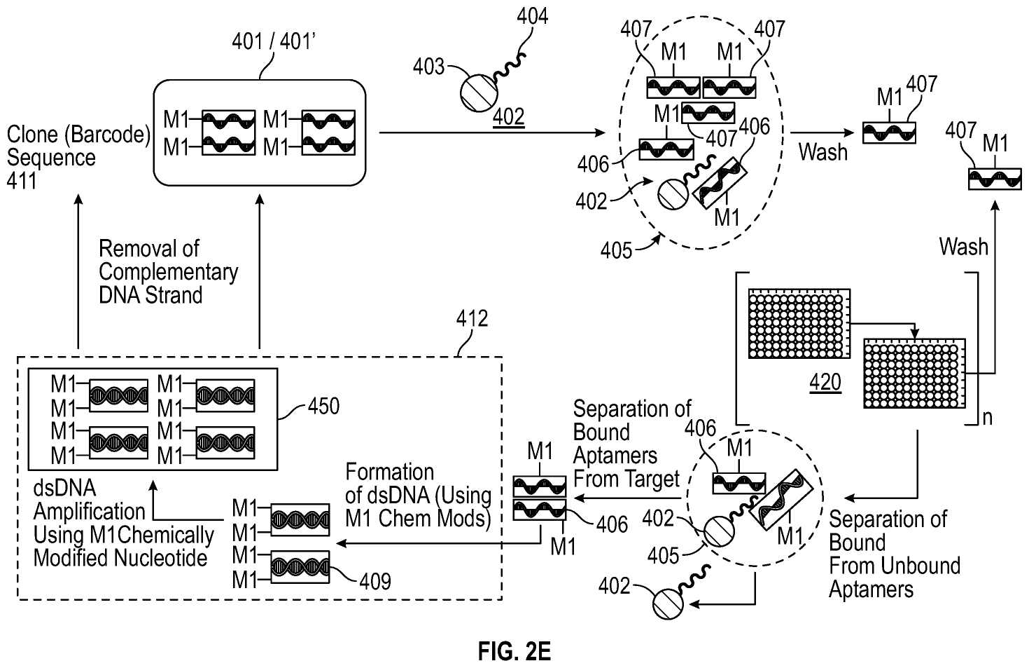

FIG. 2A illustrates an exemplary process that can be used to screen for and select affinity reagents that bind to a target analyte (e.g. bait molecule) of interest. The process is exemplified for nucleic acid aptamers but one or more steps of the process can be used for other affinity reagents. The process is also exemplified using target polypeptides as bait, but one or more steps of the process can be used for other target analytes as bait. One or more solid-phase target polypeptides 402, including a particle 403 and one or more target polypeptides 404 attached to the particle, are incubated with a library of different DNA aptamers 401 (401′ denotes a subsequent aptamer library that has been enriched by completion of a full cycle of the exemplary process). Solid-phase target polypeptides 402 may be contained in a first set of vessels, for example, in wells of a well plate. The well plate may have a solid bottom, or to aid in subsequent wash steps, the well plate may have a porous filter bottom (e.g. a mesh filter bottom). In the case of well plates having a mesh filter bottom, the filter may comprise holes or pores which are small enough to hold reaction and wash buffers at ambient pressure. However, when a positive pressure is applied to the top of the constituent wells in the well plate, or when the plate is centrifuged, or when a vacuum is applied to the bottom of the wells, liquid in the well plates may pass through the filter mesh leaving the solid-phase target polypeptides 402 in the wells of the well plate(s).

Some of the aptamers, for example, 406, bind to bait (e.g. target polypeptides) 404 via one or more epitopes on the bait to afford one or more aptamer-bait complexes associated with particle 403 in a fluid 405. Fluid 405 may also contain contaminant aptamers 407. A wash step can be used to separate contaminant aptamers 407 from solid-phase bait (e.g. target polypeptides) 402 and bound aptamers 406. Contaminant affinity reagents include, for example, those that are free in solution, or those that are non-specifically bound to a bait (such as the target polypeptide), linker, solid-phase support, well plate or any other matrix component. Other contaminant affinity reagents include those that recognize or bind non-epitope moieties of a bait molecule. For example, contaminant affinity reagents may include those that require a moiety other than a desired target epitope in order to recognize or bind the bait molecule, those that require a moiety other than a desired target epitope in order to recognize or bind the target epitope, or those that recognize or bind a moiety of the bait molecule absent presence of the desired target epitope.

Separation of bound aptamers 406 from solid-phase bait (e.g. target polypeptides) 402 affords an enriched pool of aptamers 406 having apparent binding affinity for solid-phase bait 402. Optionally, the enriched pool of aptamers 406 may be amplified in process 412. In a first step of process 412, complementary strands are synthesized for each of aptamers 406 to form double-stranded DNA (dsDNA) species 409. The dsDNA species 409 are then amplified, in this example using PCR (e.g. preparative PCR, real time PCR or quantitative PCR) to produce amplicons 410. The amplicons 410 in this example are double stranded and can be converted to single stranded form, thereby providing a new aptamer library 401′ which includes a subset of aptamers that were present in the prior library 401. Amplification is optional and can be omitted such that selected aptamers 406 are used as new aptamer library 401′. Whether produced by amplification or not, new aptamer library 401′ can be subjected to a repeat of the cycle for further refinement and selection of a desired aptamer or subset of aptamers. The cycle can be repeated, being performed at least 2, 3, 4, 5, 10, 15, 20, 25 or more times, for example, until a highly enriched subset of aptamers is produced. The aptamers produced from one or more repeats of the cycle can be sequenced to determine identities of the selected aptamers.

FIG. 2B shows a modified process where wash step 408 is replaced with process 420 for improved selection of affinity reagents. The improvement recognizes that well plates in general, have high surface areas where contaminant affinity reagents may be retained. Contaminant affinity reagents which are non-specifically bound to well plates pose a risk to future manufacture and use of desired affinity reagents. The success of a selection process, especially for aptamers and other amplifiable molecules, is benefited by minimizing carry through of contaminating molecules such as contaminant aptamer affinity reagents. Contaminant aptamer affinity reagents which are not removed from a well plate during a wash step, may overwhelm a selection process as such contaminants may be efficiently amplified by a downstream amplification process such as PCR/qPCR to the partial or full exclusion of desired aptamers.

As shown in FIG. 2B, process 420 can include a step of transferring a fluid 405 which includes solid-phase bait (e.g. target polypeptide) 402, bound aptamers 406 and contaminant aptamers 407, to a well of a multiwell plate 421. The solid-phase bait 402 is bound to at least one aptamer 406 (e.g. a target aptamer) and is associated with contaminant aptamers 407. Contaminant aptamers 407 can be separated from the solid-phase bait 402 and bound aptamers 406 by removing fluid from the well. The fluid can be removed by aspiration (e.g. via pipette), passage of the fluid through a filter, decanting or other appropriate techniques. Multiwell plates having filter bottoms can be particularly useful since fluid can be separated from the solid-phase bait 402 and bound aptamers 406 by application of positive pressure, vacuum, or gravitational force (e.g. via centrifugation of the multiwell plate). A wash fluid can then be added to the well and subsequently removed to further wash the solid-phase bait 402 and bound aptamer 406. Such wash steps can be repeated in the first multiwell plate 421 any number of times, for example, at least 2, 3, 4, 5, 10 or more washes can be performed. Whether or not washing of the solid-phase bait 402 and bound aptamer 406 is repeated in a given well of multiwell plate 421, the solid-phase bait 402 and bound aptamer 406 can be transferred to a second well such as a well in a second multiwell plate 422. The transfer can effectively separate solid-phase bait 402 and bound aptamer 406 from contaminant aptamers 407, such as aptamers that are retained by the surface of the well in multiwell plate 421. Following transfer, fluid can be removed from the solid-phase bait 402 and bound aptamer 406 using methods such as those exemplified for multiwell plate 421. Moreover, wash fluids can be delivered to solid-phase bait 402 and bound aptamer 406 in multiwell plate 422 and removed any number of times, again as exemplified for multiwell plate 421.

Following wash process 420, bound aptamers 406 can be separated from solid-phase bait (e.g. target polypeptide) 402, optionally amplified in process 412, and the amplicons 410 can be converted to single stranded form to produce an enriched aptamer library 401′. Enriched aptamer library 401′ can optionally be subjected to a subsequent cycle of the selection process. FIG. 2C shows a configuration of the selection process in which amplification is omitted. In this configuration, enriched aptamers 406 retrieved from washing process 420 and separation from solid-phase target polypeptide 402 can be used as an enriched aptamer library 401′ for further selection in the process.

Different multiwell plates may differ with respect to the chemical composition of the wells in either plate, surface treatments present in the wells of either plate, presence or absence of contaminants in the wells of either plate, shape of the wells of either plate, surface area of the wells of either plate, porosity of the wells of either plate, presence or absence of filters in the wells of either plate, porosity of the filters in the wells of either plate or the like. Multiwell plates may have been treated differently prior to use in a method set forth herein. For example, two multiwell plates may have been washed with different solutions; one multiwell plate may have been washed whereas the other was not; two multiwell plates may have been stored under different conditions such as different temperature, intensity of light, spectrum of light or humidity, or one multiwell plate may have been used in a prior selection process whereas the other has not. New or used multiwell plates may be used which were pre-treated with washing buffer containing nucleases, other enzymes which degrade nucleic acids or chemicals known to remove nucleic acids from surfaces or degrade nucleic acids. A substantial improvement in selection of affinity reagents is provided by the methods set forth herein, not merely due to washing solid-phase target analytes but also due to transfer of the solid-phase target analytes to a different vessel (e.g. different wells or well plates), where the different vessel is uncontaminated by any aptamer affinity reagents, for example, due to never having contacted aptamers from the aptamer library that is being subjected to the selection process. After one or more wash cycles of solid-phase bait or target analytes in a different well plate, the solid-phase bait or target analytes may be transferred again to another well plate and subjected to further wash cycles. This process may be repeated further until undesirable aptamer affinity reagent contamination is minimized and at a level where these contaminants minimize interferences with desirable aptamer affinity reagent amplification steps. A method set forth herein can include at least 1, 2, 3, 4, 5, 10 or more transfers of a solid-phase bait or target analyte from one vessel to another.

FIG. 2D shows a similar selection process to that shown in FIG. 2B with the addition of a negative selection step in which an enriched aptamer library 440 is contacted with a solid-phase challenge analyte 441, which includes particle 443 attached to challenge analyte 444. Challenge analyte 444 can be any substance that is suspected or known to bind to aptamers in library 440 and for which binding is not desired or acceptable. For example, the solid-phase challenge analyte can be the same type of particle as particle 403 which may optionally be attached to the same type of linker as used to attach bait (e.g. target polypeptide) 404 to particle 403. As such, the negative selection step can remove contaminant aptamers that bind to particle 403 and/or linker absent the bait 404. Other substances that can be useful challenge analytes include, but are not limited to, an analyte having an epitope that is chemically similar to a target epitope, an analyte having an epitope that is known or suspected to confound binding results obtained from a given sample, an analyte having an epitope that is known or suspected not to bind affinity reagents of the type being screened or the like. For example, a challenge polypeptide can have an epitope with a peptide sequence that is homologous, smaller or larger than a target epitope present in a target polypeptide. In another example, a challenge polypeptide can include the same amino acid sequence as the polypeptide used as bait except for omission of all or part of the desired epitope. Similarly, a challenge polypeptide can include same amino acid sequence as the polypeptide used as bait except for a replacement of all or part of the desired epitope with other amino acids, such as amino acids that are biosimilar to those they replace. Accordingly, a challenge polypeptide can include epitope flanking regions from the bait polypeptide, while lacking all or part of the epitope from the bait polypeptide. Contaminant aptamers (i.e. aptamers that are not negatively selected) can be used directly as an enriched aptamer library 401′ for subsequent cycles of the selection process. In an alternative configuration, the aptamers that passed the negative selection can be amplified in a process similar to process 412 and the resulting amplicons can be treated as the enriched aptamer library 401′ for subsequent cycles of the selection process. The negative selection process can be carried out at different stages of the process shown in FIGS. 2B and 2C. For example, negative selection can be carried out prior to the positive selection step, after the wash step and prior to the amplification step, or after the wash step and prior to repeating the selection cycle.

FIG. 2E shows a selection process for nucleic acid aptamers having chemically modified nucleotides. In this example, M1 represents one or more exogenous moieties of an aptamer. M1 may be compatible with enzymes used in amplification methods such that presence of the M1 moiety does not prevent replication or copying of the nucleic acid to which it is attached. For example, M1 may be a C1 to C5 alkyl group or hydrocarbon moiety which may facilitate hydrophobic binding interactions of the modified aptamer with a target analyte. An M1 moiety may include benzyl groups, naphthalene groups, indoles, thiophenes, or other heterocyclic moieties. In some configurations, M1 is a reactant used in a click reaction. Reagents and techniques used for Click SELEX can be used to produce chemically modified aptamers, for example, as set forth in Pfeiffer et al., Nature Protocols 13:1153-1180 (2018), which is incorporated herein by reference. Further exemplary aptamer modifications include ethyl-naphthalene; ethylphenyl; isobutyl; ethylamine; ethyl-benzothiophene; ethyl-benzylfuran; and ethylindole. Relatively small chemical groups at the 5-position of a pyrimidine base or the 7- or 8-positions of purine bases such as uridine are well known to be tolerated by many PCR polymerases. For example, Taq polymerase, Pwo or 9° North polymerase can be used to amplify nucleic acids having exogenous moieties. Routine screening methods can be used to identify other polymerases for the ability to amplify nucleic acids having a particular exogenous moiety of interest.

The cycle in FIG. 2E starts with a library of chemically modified aptamers 401 (401′ represents an enriched affinity reagent library which has already been though the cycle). Aptamer library 401 is contacted with one or more solid-phase bait (e.g. target polypeptides) 402, a resulting fluid 405 is transferred to a vessel for washing, and enriched aptamers 406 are eluted from the solid-phase bait 402 as set forth above in the context of FIG. 2B. A PCR step 412 follows in which single stranded aptamers 406 are converted to double stranded form 409 before PCR amplification. During PCR amplification, chemically modified nucleotides having M1 moieties (or precursors of M1 moieties) are used to produce amplicons comprising M1 moieties 450. The double stranded amplicons 450 are converted to single stranded form and used as an enriched amplicon library 401′ in a subsequent selection cycle. In configurations wherein an M1 precursor is attached to amplicons 450 a step can be added to convert the precursor to an M1 moiety, for example, via a click reaction.

### Aptamer Libraries

The present disclosure provides structured aptamer libraries produced by mutagenesis of a progenitor aptamer. The libraries and methods for making the libraries are exemplified herein in the context of aptamers but can be extended to other affinity reagents of interest, such as those set forth herein. A structured aptamer library can include variants of a progenitor aptamer having improved binding characteristics compared to the progenitor aptamer. For example, a variant of a progenitor aptamer can have increased specificity of binding to a target epitope of the progenitor aptamer, increased affinity for a target epitope of the progenitor aptamer (e.g. decreased K_{D}), increased probability of binding to a target epitope of the progenitor aptamer, increased rate of binding a target epitope of the starting aptamer (e.g. increased kₒₙ) or decreased rate of binding a target epitope of the progenitor aptamer (e.g. decreased k_{off}). A structured aptamer library can include variants of a progenitor aptamer that recognize target epitopes that are not substantially recognized by the progenitor aptamer.

The present disclosure provides a method for obtaining an aptamer variant. The method can include steps of: (a) randomly mutating a progenitor aptamer, thereby producing a library of aptamer variants; (b) contacting the library of aptamer variants with a solid-phase target molecule, thereby forming a solid-phase complex including the target polypeptide of the solid support bound to an aptamer variant from the library; and (c) separating the solid-phase complex from the fluid phase, thereby obtaining an aptamer variant having affinity for the target molecule.

A method for obtaining an aptamer variant from a progenitor aptamer can produce an aptamer variant having higher affinity for a target epitope compared to the affinity of the progenitor aptamer for the target polypeptide. The affinity can be characterized according to any of a variety of binding properties set forth herein. In some cases, the progenitor aptamer has no substantial affinity for the target polypeptide. Accordingly, a method set forth herein can produce variant aptamers having substantially different binding targets compared to its progenitor aptamer.

Random mutagenesis can be carried out using any of a variety of techniques. Optionally, random mutagenesis PCR can be performed using nucleotide analogs that are known or suspected of causing mutations. Nucleotide analogs can be chosen based on the types of mutations expected. For example, nucleotide analogs can cause transition mutations. Transition mutations occur when a pyrimidine base (e.g. thymine [T] or cytosine [C]) substitutes for another pyrimidine base or when a purine base (e.g. adenine [A] or guanine [G]) substitutes for another purine base. Nucleotide analogs that cause transversion mutations are also useful. Transversion mutations occur when a purine (e.g. A or G) is changed for pyrimidine (T or C), or vice versa. Optionally, one or more nucleotide analogs that are present in a random mutagenesis PCR can cause both transition and transversion mutations. Other random mutagenesis methods can be used such as use of error-prone polymerases to amplify a progenitor sequence, use of amplification conditions that induce mutations, use of doped primers for amplification, or the like. Exemplary random mutagenesis PCR techniques are set forth in Example V, herein. Methods that shuffle or recombine nucleic acid fragments can also be used such as nonhomologous random recombination. See, for example, Bittker et al., Nat Biotechnol., 20:1024-1029 (2002), which is incorporated herein by reference.

Random mutations can be introduced by performing consecutive rounds of random mutagenesis on a progenitor sequence. For example, a progenitor sequence can be amplified via at least 2, 3, 4, 5, 6 or more consecutive rounds of random mutagenesis PCR. In some cases, it can be useful to limit the number of consecutive rounds of random mutagenesis performed. For example, subjecting a progenitor sequence to fewer rounds of mutagenesis may produce a desired average number of mutations per variant produced or may minimize unwanted alterations to particular regions of the progenitor sequence. As an alternative or addition to the above lower limits on the number of consecutive PCR rounds, a progenitor sequence can be amplified via at most 6, 5, 4, 3, 2 or 1 consecutive round(s) of random mutagenesis PCR.

A random mutagenesis process used herein can be configured to produce a desired rate of mutation. For example, a random mutagenesis process can be configured to produce an average of at least 1, 2, 4, 6, 8, 10 or more mutation(s) per variant aptamer. Alternatively or additionally, a random mutagenesis process can be configured to produce an average of at most 10, 8, 6, 4, 2 or 1 mutation(s) per variant aptamer.

### Analytes for Selecting and Characterizing Affinity Reagents

The compositions, apparatus and methods set forth herein can be used in combination with any of a variety of target analytes, for example, to be used as bait in a selection process set forth herein such as SELEX. As exemplified herein, a particularly useful target analyte is a polypeptide. Other target analytes that can be used in place of a target polypeptide include, but are not limited to, a cell, organelle, virus, nucleic acid, carbohydrate, vitamin, enzyme cofactor, hormone, small molecule such as a candidate therapeutic agent, metabolite, nucleotide, amino acid, sugar, lipid, or the like.

In particular configurations, a selection method set forth herein can be used to obtain an affinity reagent that recognizes a peptidyl epitope (i.e. a polypeptide epitope) comprising two or more amino acid residues. For example, an affinity reagent used or produced in a method set forth herein can recognize an epitope that includes at least 2, 3, 4, 5, 6, 7, 8, 9, 10 or more amino acid residues in a polypeptide. Alternatively or additionally, the affinity reagent may recognize no more than 10, 9, 8, 7, 6, 5, 4, 3, or 2 amino acid residues in a polypeptide.

The amino acids of an epitope can be contiguous in the primary sequence of a polypeptide to which the affinity reagent binds. Alternatively, the amino acids of the epitope can be non-contiguous in the primary sequence of the polypeptide. Non-contiguous amino acids can, nevertheless, be proximal in the secondary, tertiary or quaternary structure of a polypeptide such that an affinity reagent interacts with the noncontiguous amino acids when bound to the polypeptide. For example, the decapeptide, NGAAALWGKR (SEQ ID NO: 1) may be a target for the discovery and selection of an affinity reagent which binds to an epitope within the decapeptide. A first epitope, E1, for a first affinity reagent may be the linear epitope -AAA-trimer of the decapeptide. The first affinity reagent may bind or recognize only the AAA region of the decapeptide and may non-covalently bind with all three alanine residues. It may also non-covalently bind to only the first and third alanines but at the same time, the second (central) alanine may be unique among amino acid residues in providing structural characteristics that position the other two alanines for interaction with the first affinity reagent. This kind of linear epitope is said to be a contiguous epitope. Continuing with the example of the decapeptide, a second affinity reagent may bind to a second epitope, E2 (-LWGK-)(SEQ ID NO: 2). In this case, the second affinity reagent may form non-covalent bonds between (or “recognize”) only three out of the four amino acid residues, for example, the L, W and K amino acid residues and bind any linear amino acid sequence in a given polypeptide comprising a -LWXK- sequence region, wherein X may be selected among a plurality of different amino acid residues or among all other amino acid residues. Here the epitopic region, LWXK is a non-contiguous or conformational epitope.

A target polypeptide that is used in a method, apparatus or composition of the present disclosure, for example, as bait to select an affinity reagent, can have a length that is equal to or larger than the epitope that is recognized by a particular affinity reagent. For example, a target polypeptide can have a length of at least 2, 3, 4, 5, 6, 7, 8, 9, 10, 15, 20, 25, 30, 40, 50 or more amino acid residues. Alternatively or additionally, a target polypeptide can have a length of no more than 50, 40, 30, 25, 20, 15, 10, 9, 8, 7, 6, 5, 4, 3, or 2 amino acid residues. The epitope of a polypeptide can include at least 2, 3, 4, 5, 6, 7, 8, 9, 10 or more amino acid residues. Alternatively or additionally, the epitope can include no more than 10, 9, 8, 7, 6, 5, 4, 3, or 2 amino acid residues.

Affinity reagents of this disclosure may be chosen for an ability to bind to a desired epitope or to bind to an amino acid sequence which is related to a desired epitope, regardless of the sequence context within which the epitope occurs. For example, an affinity reagent selected to bind to a desired specific amino acid trimer, AAA, where the linear sequence AAA is a tripeptide or is a discrete unit of a larger peptide, may bind to one or more different polypeptides containing the sequence AAA. It may also bind with AGA, GAA, AAG, or other variants of the AAA sequence, such as a variant in which one or more of the A residues is replaced with an amino acid residue that is biosimilar to A. Optionally, biosimilarity can be determined, for example, in accordance with a BLOSUM62 scoring matrix. See, for example, Egertson et al., BioRxiv (2021), DOI: 10.1101/2021.10.11.463967, which is incorporated herein by reference. A method of the present disclosure can be configured to select for affinity reagents that bind to the trimeric AAA epitope by using a target polypeptide having the AAA trimer as bait. Optionally, the method can employ a negative selection using challenge polypeptides having the AGA, GAA, AAG or other variants of the desired epitope to select against binding reagents that recognize the variants.

A plurality of polypeptides in which all members have identical length and sequence composition can be used as bait in a selection method set forth herein. For example, a bait polypeptide can have a structure αZβ, wherein Z is an epitope region, a is an optional amino flank of the epitope region, and β is an optional carboxyl flank of the epitope region. In some cases, a plurality of polypeptides used herein, for example as bait, can have a region of identical sequence and a region of variable sequence. Taking again the example of a plurality of polypeptides having sequence αZβ, epitope region Z can have an amino acid sequence that is the same for all members of the plurality, and one or both of sequences α and β can vary for target polypeptide molecules that compose the bait. The α and/or β sequences can vary, for example, with respect to length or amino acid sequence composition. Independent of any sequence variability, one or both of α and β may have a length of zero amino acids, one amino acid, or a sequence of two or more amino acids. The sequence of α or β can be at least 1, 2, 3, 4, 5, 6, 7, 8, 9, 10 or more amino acids. Alternatively or additionally, the sequence of α or β can be at most 10, 9, 8, 7, 6, 5, 4, 3, 2 or 1 amino acids. The sequence composition of α can be the same as the sequence composition of β, or the two regions can differ with regard to sequence composition.

A target polypeptide or other target analyte can include a chemical linker, for example, to facilitate attachment to a solid-phase support, particle or other object. At least one of α and β in a target polypeptide having sequence αZβ may include a chemical linker. A chemical linker may include any of a variety of chemical moieties, connecting one relevant moiety of α molecule to another relevant moiety of α molecule or solid-phase support. A chemical linker or other exogenous moiety can be attached to a polypeptide at the amino terminus, at the carboxy terminus or at a reactive moiety of an amino acid side chain. A linker may be a polymer such as a polyethylene glycol (PEG) moiety or a PEG polymer chain comprising one or more monomers, such as monomeric ethylene glycol groups present in PEG. A polymer linker may consist of at least 2, 3, 4, 5, 6, 7, 8, 9, 10, 15, 20, 30, 40, 50 or more monomers, such as monomeric ethylene glycol subunits present in PEG. A linker may have a straight, branched or cyclic chain. The chain can be, for example, a carbon chain. A linker can include a peptide or nucleic acid moiety. Other useful linkers are set forth, for example, in US Pat. App. Pub. No. 2021/0101930 A1; U.S. Pat. No. 11,203,612; US Pat. App. Pub. No. 2022/0162684 A1; or U.S. patent application Ser. No. 17/692,035, each of which is incorporated herein by reference.

Polypeptides of the present disclosure, such as solid-phase polypeptides used as bait, may include an amino terminal modification or carboxy terminal modification, for example, capping of an amino-terminus with an acetyl group or of α carboxy-terminus as a methyl ester or amide. Alternatively or additionally, modifications can occur at amino acid residues within a target polypeptide, for example, in one or more epitope regions or regions flanking an epitope. Polypeptides may be modified to remove a charge, for example, carboxy-terminal amidation or amino-terminal acetylation can remove a negative and a positive charge from a peptide in the physiological pH range.

A polypeptide of the present disclosure, such as a bait polypeptide, can include one or more of the twenty essential amino acids. For example, a polypeptide can include an A, R, N, D, C, Q, E, G, H, I, L, K, M, F, P, S, T, W, Y or V amino acid. For example, a polypeptide can include an amino acid having an aliphatic R group (e.g. G, A, V, L, I or P), a polar neutral R group (e.g. S or T), an amide-containing R group (e.g. N or Q), an aromatic R group (e.g. F, Y or W), a charged R group (e.g. D, E, H, K, or R), an anionic R group (e.g. D or E), or a cationic R group (e.g. H, K or R). Optionally, one or more of the amino acids or classes of amino acids set forth herein, can be absent at one, more than one, or all amino acids in a polypeptide of the present disclosure.

A polypeptide of the present disclosure can include a modified version of one of the twenty essential amino acids. For example, one or more of the amino acid positions in a polypeptide set forth herein can have an R group that includes a post-translational modification. A post-translational modification may be one or more of myristoylation, palmitoylation, isoprenylation, prenylation, farnesylation, geranylgeranylation, lipoylation, flavin moiety attachment, Heme C attachment, phosphopantetheinylation, retinylidene Schiff base formation, dipthamide formation, ethanolamine phosphoglycerol attachment, hypusine, beta-Lysine addition, acylation, acetylation, deacetylation, formylation, alkylation, methylation, C-terminal amidation, arginylation, polyglutamylation, polyglyclyation, butyrylation, gamma-carboxylation, glycosylation, glycation, polysialylation, malonylation, hydroxylation, iodination, nucleotide addition, phosphoate ester formation, phosphoramidate formation, phosphorylation, adenylylation, uridylylation, propionylation, pyrolglutamate formation, S-glutathionylation, S-nitrosylation, S-sulfenylation, S-sulfinylation, S-sulfonylation, succinylation, sulfation, glycation, carbamylation, carbonylation, isopeptide bond formation, biotinylation, carbamylation, oxidation, reduction, pegylation, ISGylation, SUMOylation, ubiquitination, neddylation, pupylation, citrullination, deamidation, elminylation, disulfide bridge formation, isoaspartate formation, and racemization. A post-translational modification may occur at a particular type of amino acid. For example, a phosphoryl moiety can be present on a serine, threonine, tyrosine, histidine, cysteine, lysine, aspartate or glutamate; an acetyl moiety can be present on an N-terminus or lysine; a serine or threonine residue can have an O-linked glycosyl moiety; an asparagine can have an N-linked glycosyl moiety; a proline, lysine, asparagine, aspartate or histidine can be hydroxylated; an arginine or lysine can be methylated or a ubiquitin can be attached to a lysine or an N-terminal methionine. Optionally, modified versions of essential amino acids, such as one or more of those set forth herein, can be absent at one, more than one, or all amino acids in a polypeptide of the present disclosure.

A polypeptide of the present disclosure can include a non-naturally occurring amino acid. For example, a polypeptide can include an analog of α naturally occurring moiety that is added by post-translational modification in vivo. Other moieties that can be present include, but are not limited to, label moieties such as those that produce detectable signals (e.g. luminophores or other optical labels) or those that encode information (e.g. nucleotide sequences); ligand moieties such as those that bind to receptors; or reactive moieties such as those that form covalent linkages with reactive moieties on other substances or objects. For example, an αZβ polypeptide may be modified with a linker and a functional group. The molecule may be of the structure F-L-αZβ, where F is a functional group and L is a linker. A molecule may be of the structure αZβ-L-F, where F is a functional group and L is a linker. Optionally, the α and β portions may each be glycine, or may each be one or more glycine residues. Amino acid residues may be modified to alter their aptagenicity, for example, amino acid residues may be altered by adding an atom or moiety with an overall positive charge or negative charge. A hydrophobic group may be added, a sugar moiety may be added or other such modifications so as to increase chemical diversity, water solubility or hydrophobicity. Optionally, non-naturally occurring amino acids, such as one or more of those set forth herein, can be absent at one, more than one, or all amino acids in a polypeptide of the present disclosure.

A polypeptide used herein, for example, in an affinity reagent selection method, can be synthesized using any of a variety of methods known in the art including, for example, chemical synthesis, solid phase chemical synthesis, fluid phase chemical synthesis, genetic engineering, recombinant gene expression or any combinations of these methods. Several commercial platforms exist for polypeptide synthesis, such as the MultiPep RSi synthesizer (Intavis, Germany). Polypeptides may be synthesized using liquid phase or solid phase methods. Synthesized polypeptides may be verified using any known method for polypeptide analysis. For example, polypeptides may be verified using Mass spectrometry, Matrix Assisted Laser Desorption/Ionization Time of Flight Mass spectrometry (MALDI-TOF), Matrix Assisted Laser Desorption/Ionization, AMS (Accelerator Mass Spectrometry), Gas Chromatography-MS, Liquid Chromatography-MS, Inductively Coupled Plasma-Mass spectrometry (ICP-MS), Isotope Ratio Mass Spectrometry (IRMS), Ion Mobility Spectrometry-MS, Tandem MS, Thermal Ionization-Mass Spectrometry (TIMS), nuclear magnetic resonance (NMR), or Spark Source Mass Spectrometry (SSMS). Concentration of the synthesized peptides may also be assessed by spectroscopy. Some methods described above are described in more detail in PCT application, WO2020106889A1 which is incorporated herein by reference.

A target analyte can be attached to a particle, bead, solid-phase support or other object. An object to which a target analyte is attached can be separable from a vessel in which it is contained for at least part of α method set forth herein. Thus, a bead, particle or other object need not adhere to the vessel. Rather, the object can be mobile within the vessel. Exemplary compositions for a particle, bead, solid-phase support or other object herein include, but are not limited to, plastics, ceramics, glass, polystyrene, melamine, methylstyrene, acrylic polymers, paramagnetic materials, thoria sol, carbon graphite, agarose, titanium dioxide, latex or cross-linked dextrans such as Sepharose™, cellulose, nylon, cross-linked micelles and Teflon™ or other materials set forth in “Microsphere Detection Guide” from Bangs Laboratories, Fishers Ind., which is incorporated herein by reference. Structured nucleic acid particles, for example, those that include nucleic acid origami structures can be useful. The shape of α particle or bead can correspond to any of a wide variety of different forms and shapes. For example, they can be symmetrically shaped (e.g. spherical or cylindrical) or irregularly shaped (e.g. controlled pore glass). In addition, particles or beads can be, for example, porous, thus increasing the surface area available for capture or non-porous. Exemplary sizes for beads used herein can range from the nanometer to millimeter range. For example, a bead or particle can have a diameter of at least about 50 nm, 100 nm, 1 μm, 10 μm, 50 μm, 100 μm, 250 μm, 1 mm or more. Alternatively or additionally, a bead or particle can have a diameter of at most about 1 mm, 250 μm, 100 μm, 50 μm, 10 μm, 1 μm, 100 nm, 50 nm or less.

Bait such as a polypeptide or other target analyte can be attached to a solid-phase support, particle or other object. For example, a polypeptide or other analyte of interest can be attached to a solid-phase support, particle or object using click chemistry. Exemplary click reactions are set forth in WO 2019/195633A1; U.S. Pat. No. 11,203,612; US Pat. App. Pub. No. 2022/0162684 A1; or U.S. patent application Ser. No. 17/692,035, each of which is incorporated herein by reference. Exemplary click reactions may include metal-catalyzed azide-alkyne cycloaddition, strain-promoted azide-alkyne cycloaddition, strain-promoted azide-nitrone cycloaddition, strained alkene reactions, thiol-ene reaction, Diels-Alder reaction, inverse electron demand Diels-Alder reaction, [3+2] cycloaddition, [4+1] cycloaddition, nucleophilic substitution, dihydroxylation, thiol-yne reaction, photoclick, nitrone dipole cycloaddition, norbornene cycloaddition, oxanobornadiene cycloaddition, tetrazine ligation, and tetrazole photoclick reactions. Exemplary functional groups that can be present on a substance that is to be linked to another may include alkenes, alkynes, azides, allenes, epoxides, amines, thiols, nitrones, isonitriles, isocyanides, aziridines, activated esters, and tetrazines. A receptor (e.g. (strept)avidin) that is attached to a polypeptide or particle can be bound to a ligand (e.g. biotin) that is attached to a particle or polypeptide, thereby attaching polypeptide to particle.

A target analyte can be attached to an object such as a bead, solid-phase support or particle using any of a variety of chemistries. Optionally, a target analyte can be covalently attached to an object such as a bead or particle. Covalent attachment can occur via an uninterrupted chain of covalent bonds between the target analyte and the object. Optionally, covalent attachment of target analytes to beads, particles or other objects can be achieved using cyanogen bromide coupling, N-hydroxysuccinimide ester couplings (e.g. where the NHS ester is part of the beads and target polypeptides are coupled through lysine residues or N-termini), click chemistry, or other methods used to couple peptides to solid-phase supports.

In some configurations, surfaces of α solid-phase support, bead, particle or other object may be modified to allow or enhance covalent or non-covalent attachment of target analytes or other molecules. The particle and process for molecule attachment are preferably stable for repeated binding, washing, imaging and eluting steps. In some cases, surfaces may be modified to have a positive or negative charge. In some cases, surfaces may be functionalized by modification with specific functional groups, such as maleic or succinic moieties, or derivatized by modification with a chemically reactive group, such as amino, thiol, or acrylate groups, such as by silanization. Suitable silane reagents include aminopropyltrimethoxysilane, aminopropyltriethoxysilane and 4-aminobutyltriethoxysilane. Surfaces may be functionalized with N-Hydroxysuccinimide (NHS) functional groups. Glass surfaces can also be derivatized with other reactive groups, such as acrylate, epoxy, or thiol, using, e.g., epoxysilane, acrylatesilane, mercaptosilane, or acrylamidesilane.

Bioconjugation may be used to form a covalent bond between a target molecule and an object (e.g. particle, bead or solid-phase support). Bioconjugation may be formed, for example, via chemical conjugation, enzymatic conjugation, photo-conjugation, thermal-conjugation, or a combination thereof. (Spicer, et al. Chemical Reviews., 2018, 118, Pgs. 7702-7743, or Hermanson, Bioconjugate Techniques, Academic Press; 3rd Edition, 2013, each of which is incorporated herein by reference). In some cases, both the object (e.g. particle, bead or solid-phase support) and the target analyte can be functionalized. Functionalizing both partners may improve the efficiency or speed of α conjugation reaction. For example, a sulfhydryl group (—SH) or amine (—NH 2) of α chemically active site of α target protein may be functionalized to allow for greater reactivity or efficiency of α conjugation reaction. Any of α variety of sulfhydryl-reactive (or thiol-reactive) or amine conjugation chemistries may be used to couple chemical moieties to sulfhydryl or amine groups. Examples include, but are not limited to, use of haloacetyls, maleimides, aziridines, acryloyls, arylating agents, vinylsulfones, pyridyl disulfides, TNB-thiols and/or other sulfhydryl-reactive/amine-reactive/thiol-reactive agents. Many of these groups conjugate to sulfhydryl groups through either alkylation (e.g., by formation of α thioether or amine bond) or disulfide exchange (e.g., by formation of α disulfide bond).

Bioconjugation can be accomplished in part by a chemical reaction of α chemical moiety or linker molecule with a chemically active site on an analyte or solid-phase support. The chemical conjugation may proceed via an amide formation reaction, reductive amination reaction, N-terminal modification, thiol Michael addition reaction, disulfide formation reaction, copper(I)-catalyzed alkyne-azide cycloaddition (CuAAC) reaction, strain-promoted alkyne-azide cycloaddition reaction (SPAAC), Strain-promoted alkyne-nitrone cycloaddition (SPANC), invers electron-demand Diels-Alder (IEDDA) reaction, oxime/hydrazone formation reaction, free-radical polymerization reaction, or a combination thereof. Enzyme-mediated conjugation may proceed via transglutaminases, peroxidases, sortase, SpyTag-SpyCatcher, or a combination thereof. Photoconjugation may proceed via photoacrylate cross-linking reaction, photo thiol-ene reaction, photo thiol-yne reaction, or a combination thereof. In some cases, conjugation may proceed via non-covalent interactions, these may be through self-assembling peptides, binding sequences, receptor-ligand pairs (e.g. streptavidin-biotin, lectin-carbohydrate, etc.) host-guest chemistry, complementary nucleic acids, or a combination thereof. Other attachment chemistries known in the art can be used including, for example, those set forth in WO 2019/195633 A1; US Pat. App. Pub. No. 2021/0101930 A1; U.S. Pat. No. 11,203,612; US Pat. App. Pub. No. 2022/0162684 A1; or U.S. patent application Ser. No. 17/692,035, each of which is incorporated herein by reference.

Optionally, an object, such as a particle or solid support, can be attached to a plurality of target molecules (e.g. target polypeptides or bait polypeptides). In some configurations, the plurality of target molecules can be homogenous. For example, polypeptides that are attached to a particle solid support or other object can be homogenous with respect to their amino acid sequences. Such beads can be useful for binding affinity reagents that recognize an epitope in the target molecules and separating the bound affinity reagents from affinity reagents that do not recognize the epitope. In some cases, an object that is attached to a plurality of target molecules can be bound to a plurality of affinity reagents. The affinity reagents can have the same composition as each other. However, the affinity reagents need not have the same composition. For example, affinity reagents that differ in composition can nevertheless have affinity for the same epitope. This can be the case for beads used in a SELEX process or other selection process, wherein a library of affinity reagents includes several variants that bind to a target epitope.

Any of α variety of vessels can be used in a method set forth herein. Individual vessels can be useful, for example, when a relatively low plexity of target analytes (e.g. bait) or relatively small library of affinity reagents is used. Alternatively, a multi-vessel format can be used, for example at higher plexity or for larger affinity reagent libraries. Multiwell plates can be particularly useful. Transfer of particles or other objects from one vessel to another can be configured to move the particles or other objects from one well of α multiwell plate to another well of the plate. Alternatively, transfer can happen between different multiwell plates such that an object, such as a particle, is transferred from a well of α first multiwell plate to a well of α different multiwell plate.

The vessel from which an object, such as a particle, is transferred can have the same, similar or different composition in comparison to the vessel where the object is transferred. The comparison can refer to the composition of the vessels, the surface properties of the vessels, history of use for the vessels, shape of the vessels, surface area of the vessels, porosity of the vessels, presence or absence of filters in the vessels, pore size (or mesh size) for filters in the vessels, or the like.

A vessel used herein can have any of a variety of compositions including, for example, compositions set forth herein in the context of particles, beads or solid-support substrates. For example, a vessel such as a multiwell plate can be composed of glass, silica, quartz or polymeric material. Exemplary polymeric materials include, but are not limited to, polypropylene, polyethylene, polyamide, polyester, polytetrafluoroethylene (PTFE), polyethylene terephthalate (PET) or polycarbonate.

A vessel can include a porous filter through which fluid can be passed to separate the fluid from a bead, particle or solid phase support. For example, a multiwell plate can include wells having filter bottoms. A porous filter can have a pore size that is too small to pass objects such as beads or particles, but large enough to pass fluid. For example, the pore size can be at least 0.01 μm, 0.05 μm, 0.1 μm, 0.5 μm, 1 μm, 5 μm, 10 μm, 50 μm, 100 μm, or larger. Alternatively or additionally, the pore size can be at most about 100 μm, 10 μm, 5 μm, 1 μm, 0.5 μm, 0.1 μm, 0.05 μm, 0.01 μm, or smaller. A porous filter can be made of a mesh or other material such as polytetrafluoroethylene (PTFE) mesh, glass mesh or polyethylene terephthalate (PET) mesh.

An affinity reagent of the present disclosure can have affinity for a target analyte, or epitope of the target analyte that is considered “high affinity,” “medium affinity,” or “low affinity.” A binding affinity—can be considered “high affinity” if the interaction has an equilibrium dissociation constant ((K_{D}) of less than about 100 nM, “medium affinity” if the interaction has a dissociation constant between about 100 nM and 1 mM, and “low affinity” if the interaction has a dissociation constant of greater than about 1 mM. The dissociation constant for binding of an affinity reagent to a target analyte or epitope thereof can be less than 10⁻³ M, 10⁻⁴ M, 10⁻⁶ M, 10⁻⁸ M, 10⁻¹⁰ M, 10⁻¹² M, 10⁻¹⁴ M or lower. Binding affinity can be described in terms known in the art of biochemistry such as equilibrium dissociation constant, equilibrium association constant (K_{A}), association rate constant (kₒₙ), dissociation rate constant (k_{off}) and the like. See, for example, Segel, Enzyme Kinetics John Wiley and Sons, New York (1975), which is incorporated herein by reference in its entirety. An affinity reagent can be characterized in terms of the probability of binding to a target analyte or epitope thereof. Such a characterization can be particularly useful when evaluating the results of binding reactions detected at single molecule resolution such as those set forth in U.S. Pat. No. 10,473,654 or 11,282,585; or Egertson et al., BioRxiv (2021), DOI: 10.1101/2021.10.11.463967, each of which is incorporated herein by reference. The probability of can be characterized as a probability of an affinity reagent binding to a target analyte or epitope thereof can be at least 0.25, 0.5, 0.51, 0.75, 0.9, 0.99 or higher (on a scale of 0 to 1).

The present disclosure provides polypeptides having epitopes within structural motifs that improve binding of the epitopes to affinity reagents. The polypeptides can be advantageously employed as bait molecules for selection of affinity reagents that are specific for the epitopes relative to other portions of the polypeptide. A polypeptide of the present disclosure can include a polypeptide epitope and a flanking moiety on one or both sides of the epitope. A flanking moiety can provide three-dimensional structure to the polypeptide, thereby orienting the epitope to bind an affinity reagent such that the affinity reagent need not interact with the flanking regions or other regions of the polypeptide. Typically, flanking regions will be present on both sides of an epitope region, for example, a first flanking region (α) being on the amino terminal side of the epitope region (Z) and a second flanking region (β) being on the carboxyl terminal side of the epitope region, as occurs in polypeptide sequence αZβ. In the examples herein, epitope region Z can include one or more amino acids, and each of α and β can include one or more amino acids. In some cases, an epitope can be located at one end of α target polypeptide, for example, epitope Z being at the amino terminus or carboxyl terminus of the target polypeptide, as occurs in the sequence αZ or Zβ. As such, a target polypeptide can have a single contiguous flanking region or two contiguous flanking regions. More than two flanking regions can be present, for example, in configurations that include multiple epitopes, wherein one or more scaffold regions intervene two or more epitopes.

A polypeptide of the present disclosure can include an amino acid sequence selected from the group consisting of SGASZSGAS; SESZNLYY; NDFZTISR; SGSZNLYY; and NGFZTISR, wherein Z designates a region that can include any of a variety of amino acid compositions and lengths. Optionally, Z can include an epitope and the other amino acids can provide flanking regions. The flanking regions can be configured to take on a structural motif that influences the ability of an affinity reagent to recognize or bind to an epitope in Z. Exemplary compositions and lengths for Z are set forth herein for example in Table 1 and Table 2.

A position identified in a polypeptide herein as Z can have a length of at least 3, 4, 5, 6, 7, 8, 9, 10 or more amino acids. Alternatively or additionally, Z can have a length of at most 10, 9, 8, 7, 6, 5, 4, 3 or fewer amino acids. A variable amino acid position can be indicated by “X”. Accordingly, Z can have an amino acid sequence of, XXX, XXXX, XXXXX, XXXXXX, XXXXXXX, XXXXXXXX, XXXXXXXXX, or XXXXXXXXXX. The overall length for the polypeptide can be, for example, at least 10, 15, 20, 25, 30, 35, 40, 50 or more amino acids. Alternatively or additionally, the overall length for the polypeptide can be at most 50, 40, 35, 30, 25, 20, 15, 10 or fewer amino acids.

A position identified in a polypeptide herein as X or in Z can include an A, R, N, D, C, Q, E, G, H, I, L, K, M, F, P, S, T, W, Y or V amino acid. For example, an amino acid in X or Z can have an aliphatic R group (e.g. G, A, V, L, I or P), a polar neutral R group (e.g. S or T), an amide-containing R group (e.g. N or Q), an aromatic R group (e.g. F, Y or W), a charged R group (e.g. D, E, H, K, or R), an anionic R group (e.g. D or E), or a cationic R group (e.g. H, K or R). Optionally, one or more of the amino acids or classes of amino acids set forth herein, can be absent at one, more than one, or all amino acids in a polypeptide of the present disclosure.

A position identified in a polypeptide herein as X or in Z can include a modified version of one of the twenty essential amino acids. For example, one or more of the amino acid positions can have an R group that includes a post-translational modification including, but not limited to those set forth herein. Optionally, modified versions of essential amino acids, such as one or more of those set forth herein, can be absent at a position identified in a polypeptide herein as X or in Z.

A polypeptide of the present disclosure can include a non-naturally occurring amino acid. For example, a position identified in a polypeptide herein as X or in Z can include an analog of α post-translational modification. Other moieties that can be present include, but are not limited to, label moieties such as those that produce detectable signals (e.g. luminophores or other optical labels) or those that encode information (e.g. nucleotide sequences); ligand moieties such as those that bind to receptors; or reactive moieties such as those that form covalent linkages with reactive moieties on other substances or objects. Optionally, non-naturally occurring amino acids, such as one or more of those set forth herein, can be absent at a position identified in a polypeptide herein as X or in Z.

A polypeptide set forth herein can form a complex with an affinity reagent. Any of α variety of affinity reagents set forth herein or known in the art can be present in the complex. Optionally, a complex can include no more than one polypeptide and no more than one affinity reagent. However, in some cases an affinity reagent can bind to multiple polypeptides. Alternatively or additionally, multiple affinity reagents can bind a polypeptide. In some cases. a complex can include multiple affinity reagents and multiple polypeptides.

A polypeptide of the present disclosure can include two or more epitopes. For example, a region designated as Z in a polypeptide herein can include at least 1, 2, 3, 4, 5 or more epitopes. Neighboring epitopes can be immediately adjacent to each other such that no amino acids intervene between the epitopes. Alternatively, neighboring epitopes can be separated by one or more amino acids. A region having two or more epitopes, such as a region designated as Z herein, can be flanked on one or both sides by a flanking sequence set forth herein. Optionally, flanking regions of α region having multiple epitopes can form a structural motif that improves interaction of an affinity reagent with one, more than one or all of the epitopes.

Exemplary polypeptide sequences having two or more epitopes include

(SEQ ID NO: 48)
SESZ₁SGASZ₂NLYY;
(SEQ ID NO: 49)
SGSZ₁SGASZ₂NLYY;
(SEQ ID NO: 50)
NDFZ₁SGASZ₁TISR
and
(SEQ ID NO: 51)
NGFZ₁SGASZ₂TISR.

Optionally, the regions Z₁ and Z₂ are epitopes. The sequences of two or more epitopes in a given polypeptide sequence, such as Z₁ and Z₂, can be the same as each other or different from each other. In some configurations, one of the epitopes (e.g. Z₂) functions as a target epitope for an affinity reagent of interest and the other epitope (e.g. Z₁) is an adjunct to the target epitope. An adjunct epitope can enhance or facilitate binding of an affinity reagent to a target epitope. For example, the adjunct epitope can be well suited to binding affinity reagents of the type being screened for binding to the target epitope. More specifically, the adjunct epitope can be aptagenic and useful for recruiting aptamers in a method for selecting an aptamer that binds the target epitope. Similarly, the adjunct epitope can be immunogenic and useful for recruiting antibodies in a method for selecting an antibody that binds the target epitope.

A method of the present disclosure can be configured to select for affinity reagents that are minimally influenced by flanking regions α and β when recognizing epitope Z in a polypeptide having the sequence αZβ. In this example, a selection method can employ a negative selection step using challenge polypeptide(s) having the sequence αβ (i.e. the flanking sequences absent the epitope Z) or having the sequence αUβ, wherein U has a different amino acid composition than Z. Optionally, U can have the same number of amino acids as Z, but identical sequence length is not necessary for U and Z.

A selection method can include steps of (a) contacting a fluid phase with a solid-phase support, wherein the fluid phase includes a plurality of different affinity reagents, wherein the solid-phase support is attached to a target polypeptide, thereby forming a solid-phase complex including the target polypeptide of the solid support bound to a first affinity reagent of the plurality of different affinity reagents; and (b) separating the solid-phase complex from the fluid phase. Optionally, the target polypeptide has a sequence selected from the group consisting of WGGGSGASZSGAS; SESZNLYY; NDFZTISR; SGSZNLYY; and NGFZTISR, wherein Z designates a region of variable amino acid composition and length. Other target polypeptides can be used as well including, for example, those set forth herein such as in Table 1 or Table 2.

A solid-phase complex that is separated in a selection method can be dissociated to obtain the affinity reagent from the complex. The affinity reagent can then be used in a downstream application, for example, as a reagent in a binding assay, a reagent for capturing a target molecule of interest, or as an input to further rounds of α selection method set forth herein. Prior to the dissociation step, the solid-phase complex can optionally be washed to remove unbound affinity reagents or other contaminants that had carried over from the fluid-phase.

In some configurations, an affinity reagent that is used in a selection method includes a nucleic acid. For example, a nucleic acid aptamer can be bound in the solid-phase complex, separated from the fluid phase and dissociated from the solid-phase complex. Optionally, a nucleic acid of an affinity reagent can be amplified to produce amplicons of the affinity reagent, for example, after the separation step, after the dissociation step or after a wash step. Any of a variety of amplification techniques can be used including, but not limited to, those set forth herein or known in the art. Amplicons can be used in a downstream application, for example, as a reagent in a binding assay, a reagent for capturing a target molecule of interest, or as an input to further rounds of a selection method set forth herein.

Accordingly, a selection method can include steps of (a) contacting a fluid phase with a solid-phase support, wherein the fluid phase includes a plurality of different affinity reagents, wherein the affinity reagents include nucleic acids, and wherein the solid-phase support is attached to a target polypeptide, thereby forming a solid-phase complex including the target polypeptide of the solid support bound to a first affinity reagent of the plurality of different affinity reagents; (b) separating the solid-phase complex from the fluid phase; (c) dissociating the first affinity reagent from the solid-phase complex after step (b), and (d) amplifying the first affinity reagent to produce a plurality of amplicons of the first affinity reagent.

An affinity reagent, or amplicon thereof, that is obtained from a selection method can be used as an input to a further round of solid-phase capture and separation. In some cases, the further round can be carried out for the affinity reagent or amplicon absent any substantial change to its composition.

Accordingly, a selection method can include steps of (a) contacting a fluid phase with a solid-phase support, wherein the fluid phase includes a plurality of different affinity reagents, and wherein the solid-phase support is attached to a target polypeptide, thereby forming a solid-phase complex including the target polypeptide of the solid support bound to a first affinity reagent of the plurality of different affinity reagents; (b) separating the solid-phase complex from the fluid phase; (c) dissociating the first affinity reagent from the solid-phase complex after step (b), and (d) contacting a second fluid phase with a second solid-phase support, wherein the second fluid phase comprises the first affinity reagent dissociated from the solid-phase complex or an amplicon of the first affinity reagent, thereby forming a second solid-phase complex comprising the target polypeptide of the second solid support bound to the first affinity reagent dissociated from the solid-phase complex or the amplicon thereof.

Alternatively, the affinity reagent, or amplicon thereof, can be modified prior to a second round of selection. For example, the affinity reagent can be mutated or otherwise changed to produce a library of modified affinity reagents, or modified amplicons, and the modified species can be introduced as inputs to a method of solid-phase capture and separation. As such, a selection method of the present disclosure can be used to evolve or mature an affinity reagent.

Accordingly, a selection method can include steps of (a) contacting a fluid phase with a solid-phase support, wherein the fluid phase includes a plurality of different affinity reagents, and wherein the solid-phase support is attached to a target polypeptide, thereby forming a solid-phase complex including the target polypeptide of the solid support bound to a first affinity reagent of the plurality of different affinity reagents; (b) separating the solid-phase complex from the fluid phase; (c) dissociating the first affinity reagent from the solid-phase complex after step (b); (d) modifying the first affinity reagent of an amplicon of the first affinity reagent to produce a modified affinity reagent; and (e) contacting a second fluid phase with a second solid-phase support, wherein the second fluid phase comprises the modified affinity reagent, thereby forming a second solid-phase complex comprising the target polypeptide of the second solid support bound to the modified affinity reagent.

In some configurations, a further round of α selection method can be carried out using a target polypeptide having an epitope that is the same as the epitope in the target polypeptide that was used in a prior round of the selection method. For example, a first round of selection can use a target polypeptide having a sequence selected from the group consisting of WGGGSGASZSGAS; SESZNLYY; NDFZTISR; SGSZNLYY; NGFZTISR and other sequences set forth herein such as in Table 1 or Table 2. In this example, a target polypeptide that is used in the first round (e.g. being attached to a first solid phase support) can have a sequence that is identical to some or all of the sequence for a target polypeptide used in a second round of selection (e.g. being attached to a second solid support). This configuration can be advantageous to remove contaminants or weakly binding affinity reagents that passed through the prior round of selection. Different conditions can be used in each round of selection. For example, binding conditions can be more restrictive or more selective in a second round compared to a first round. Serially increasing restrictiveness of binding conditions over repeated rounds of selection can be used in a method set forth herein, for example, to increase affinity reagent specificity. This approach can be coupled with serial modification of the affinity reagent(s) selected in each round, thereby evolving or maturing the affinity reagent(s).

In an alternative configuration, a further round of solid-phase capture and separation can be carried out using a second target polypeptide that differs from the target polypeptide that was used in a prior round. For example, the first and second target polypeptides can include the same epitope, but the two polypeptides can differ with respect to one or both epitope flanking regions. By way of more specific example, a first round of selection can use a target polypeptide having a sequence selected from the group consisting of WGGGSGASZSGAS; SESZNLYY; NDFZTISR; SGSZNLYY; NGFZTISR and other sequences set forth herein such as in Table 1 or Table 2. In this example, a first target polypeptide can be used in the first round (e.g. being attached to a first solid phase support) and can have the same composition for Z or XXX as the composition for Z or XXX in a target polypeptide used in a second round of selection (e.g. being attached to a second solid support), but the first target polypeptide can have a different amino acid sequence flanking Z or XXX compared to the amino acid sequence flanking Z or XXX in the second target polypeptide. As such, the first and second target polypeptides can have different sequences selected from among those set forth herein, such as those listed in Table 1 or Table 2. This configuration can be advantageous for selecting affinity reagents that are selective for a given epitope independent of the sequence context within which the epitope resides.

### Characterization of Affinity Reagents

The present disclosure provides a method for characterizing affinity reagents including the steps of (a) providing a library of affinity reagents; (b) performing an affinity reagent selection process, including: (i) contacting the library with solid-phase target analytes, thereby forming solid-phase complexes including the target analytes and a subset of the candidate affinity reagents, and (ii) separating the subset of candidate affinity reagents from the library, thereby obtaining a pool of selected affinity reagents; and (c) performing binding assessment process, including: (i) contacting the selected affinity reagents with test analytes, thereby forming complexes including the test analytes and a subset of the selected affinity reagents, (ii) separating the subset of selected affinity reagents from the pool, thereby obtaining a plurality of candidate affinity reagents, and (iii) detecting candidate affinity reagents of the plurality of candidate affinity reagents. The methods are particularly well suited to aptamers and will be exemplified as such. However, the methods can be used for affinity reagents other than aptamers.

A binding assessment process can be used to evaluate affinity reagents obtained from a selection process. The binding assessment process can include a step of contacting selected affinity reagents with test analytes, thereby forming complexes including the test analytes and at least a subset of the selected affinity reagents. Binding assessment can be carried out for a pool of selected affinity reagents. For example, a pool, including a variety of different affinity reagents, can be contacted with test analytes, such that the different affinity reagents are in fluid communication with each other and with the test analytes during complex formation. Individual affinity reagents need not necessarily be separated from each other when contacted with test analytes. For example, a pool of different aptamers can be contacted with test analytes to form a pool of aptamer-analyte complexes.

A binding assessment process can be performed with one or more aliquot removed from a pool of selected affinity reagents. An aliquot can include a fraction of α pool of selected affinity reagents that constitutes no more than 50%, 25%, 10%, 5%, 1%, 0.5% or less of the pool of selected affinity reagents. As such, a substantial portion of the pool of selected affinity reagents can be used in any of a variety of methods set forth herein since it is not necessarily consumed by the binding assessment process.

A pool of affinity reagents that is characterized in a method set forth herein, for example in a binding assessment process, can include at least 2, 10, 100, 1×10³, 1×10⁴, 1×10⁶, 1×10⁸, 1×10¹⁰, or more different affinity reagents. Alternatively or additionally, a pool of affinity reagents that is characterized or assessed can include at most 1×10¹⁰, 1×10⁸, 1×10⁶, 1×10⁴, 1×10³, 100, 10 or 2 different affinity reagents. The affinity reagents can differ by any of a variety of structural criteria. For example, aptamers or other nucleic acid-based affinity reagents can differ with respect to nucleic acid sequences, presence of non-natural nucleotide analogs or location of non-natural nucleotide analogs in the sequences. It will be understood that one or more steps of α binding assessment process set forth herein can be performed on an individual affinity reagent or on a plurality of affinity reagents that are resolved one from the others.

In some configurations, a binding assessment process can be configured for fluid-phase binding between affinity reagents and test analytes. For example, a binding assessment process can include a step of contacting selected affinity reagents with test analytes in fluid phase, thereby forming fluid-phase complexes including the test analytes and a subset of the selected affinity reagents. As such, the test analytes need not be immobilized on solid-phase supports and the affinity reagents need not be immobilized on solid-phase supports. However, complexes formed between affinity reagents and test analytes can subsequently be immobilized on solid-phase support(s). For example, complexes can be attached to a solid-phase support to facilitate a step of separating a binding-competent subset of selected affinity reagents from unbound affinity reagents. A complex between an affinity reagent and analyte can be attached to a solid-phase support via a moiety that is present on the analyte. For example, the moiety can be a ligand (e.g. biotin or biotin analog) that binds to a receptor (e.g. avidin, streptavidin or analog thereof) on the solid-phase support. In another example, an analyte can include a moiety that is reactive with another moiety on a solid-phase support. Useful moieties that can be used for attachment are set forth herein or in WO 2019/195633A1; U.S. Pat. No. 11,203,612, US Pat. App. Pub. No. 2022/0162684 A1; or U.S. patent application Ser. No. 17/692,035, each of which is incorporated herein by reference.

In some configurations, a binding assessment process can be configured for solid-phase binding. For example, performing a binding assessment process can include a step of contacting selected affinity reagents with immobilized test analytes, thereby forming solid-phase complexes including the test analytes and a subset of the selected affinity reagents. Thus, the test analytes can be attached to a solid-phase support when contacted with affinity reagents. The affinity reagents can be in fluid-phase when contacted with immobilized test analytes such that the affinity reagents become immobilized upon binding to the test analytes. The test analytes can be attached to solid-phase supports via covalent or non-covalent linkages. Exemplary linkages are set forth herein or in WO 2019/195633A1; U.S. Pat. No. 11,203,612, US Pat. App. Pub. No. 2022/0162684 A1; or U.S. patent application Ser. No. 17/692,035, each of which is incorporated herein by reference.

Any of α variety of solid-phase supports can be used in a process for characterizing affinity reagents. Complexes between test analytes and affinity reagents need not be spatially separated from each other on a solid-phase support, for example, when characterizing a pool of affinity reagents in bulk. However, if desired complexes can be arrayed on a solid-phase support such that they are individually distinguishable one from the others. Any of α variety of solid-phase supports can be used including, for example, those set forth herein or known in the art. Particles or beads can be particularly useful. For example, paramagnetic or magnetic particles can be readily separated from fluid-phase and optionally washed to provide convenient separation of bound affinity reagents from unbound affinity reagents. Pierce™ Streptavidin Magnetic Beads (ThermoFisher, Cat. No. 88816) are particularly useful.

Complexes that include affinity reagents and test analytes can be detected, for example, during or after being separated from unbound affinity reagents. In some configurations of the binding assessment process set forth herein, the complexes or the affinity reagents in the complexes are detected as a pool. Detection need not distinguish individual affinity reagents nor individual complexes. Moreover, detection need not distinguish different affinity reagents from each other. Rather, a binding assessment process can provide a bulk characteristic of α pool or of affinity reagents. Thus, the binding assessment process can be configured to determine an average characteristic for a pool of different affinity reagents without necessarily distinguishing the characteristics of any one affinity reagent in the pool. For example, a signal can be acquired from a pool of immobilized aptamer-analyte complexes that includes a variety of different aptamers such that signals from the different aptamers are not distinguished from each other. By way of more specific example, a pool of immobilized aptamer-analyte complexes can include a variety of different aptamers bound to immobilized test polypeptides, and the presence of aptamers can be detected based on an average signal acquired from intercalating agents that bind to the pool of aptamers. As such, the acquired signal need not distinguish one aptamer from another. Intercalating agents that produce optically detectable signals are particularly useful (e.g. SYBR gold, Thermo Fisher, Waltham, MA).

A binding assessment process can be carried out to quantify selected affinity reagents, for example, in a pool of selected affinity reagents. For example, the number of affinity reagents that bind to a test analyte can be determined. In some cases, enrichment of selected affinity reagents can be quantified relative to the number of affinity reagents that were present in a library from which the affinity reagents were selected.

In some cases, quantification can be achieved by fractionating a pool of selected affinity reagents into a plurality of aliquots; contacting each aliquot with a different amount or concentration of test analytes, respectively; and detecting the amount of affinity reagent in each aliquot that complexes with the test analytes. As set forth above and in the context of FIG. 1D, the complexes can be formed in fluid-phase and then the complexes can be captured on solid-phase. Alternatively, the complexes can be formed on solid-phase. For either configuration, the solid-phase complexes can optionally be washed to remove residual contaminants, such as unbound affinity reagents. Solid-phase complexes can be detected directly on solid-phase. Alternatively, the complexes or affinity reagents can be removed from solid-phase for subsequent detection. Returning to the example of aptamers, several aliquots of aptamers that have been obtained from a selection process can be contacted with different amounts of test polypeptides, respectively; the resulting aptamer-analyte complexes can be immobilized on solid-phase support(s); the immobilized complexes can be contacted with an intercalating agent (or other probe that distinguishes aptamers from the polypeptide analytes and solid-phase support), and the intensity of signal from the intercalating agents can be detected in each aliquot. The quantity of selected affinity reagents obtained from the selection process can then be determined, for example, from a binding curve generated from the detected aliquots.

A test analyte that is used to characterize affinity reagents obtained from a selection process will typically include an epitope that was present in a target analyte that was used as bait to select the affinity reagents. In some cases, the test analyte will be substantially identical to the target analyte. For example, a test polypeptide used for a binding assessment process can have the same amino acid sequence as a target polypeptide used for selection. However, the structure of a test analyte can differ from the structure of a target analyte. This can be the case whether or not the two structures share a common epitope. For example, a test polypeptide can include amino acid residues that are not present in a target polypeptide, or a test polypeptide can exclude amino acid residues that are in a target polypeptide. Accordingly, a test polypeptide can have a shorter or longer amino acid sequence than a target polypeptide. Moreover, one or more positions in the amino acid sequence of a test polypeptide can have a different amino acid residue when compared to the comparable position in a target polypeptide. A test analyte can also differ from a target analyte with respect to moieties that attach the analytes to solid-phase supports. For example, a test polypeptide can be attached to a solid-phase support via an amino acid residue that is different in composition and/or position compared to an amino acid that attaches a target polypeptide to a solid-phase support. Moreover, a test analyte and target analyte can be attached to solid-phase supports having the same or different composition.

In particular embodiments, a test analyte that is used to characterize affinity reagents can include an epitope that differs from the epitope that was present in a target analyte used to select the affinity reagents. One or more test analytes having epitopes with various differences compared to a target epitope can be used to characterize specificity of selected affinity reagents for epitopes. For example, affinity reagents that have been selected for binding to a trimer amino acid epitope of a polypeptide can be characterized with respect to binding test polypeptides having a different amino acid at one or more positions of the trimer epitope. By way of more specific example, an amino acid at a particular position in the epitope of a test polypeptide can be biosimilar to the amino acid at the comparable position in the epitope of a target polypeptide. Biosimilarity can be determined, for example, in accordance with the BLOSUM62 scoring matrix. Alternatively or additionally to differences in epitope structures, test analytes can differ from target analytes at a region that flanks an epitope. For example, a test polypeptide can differ from a target polypeptide at one or both of (1) the amino acid(s) adjacent to the amino terminus of the epitope or (2) the amino acid(s) adjacent to the carboxyl terminus of the epitope.

It will be understood that a test analyte can have any of a variety of structures or functions set forth herein in the context of target analytes. For example, a test polypeptide can include an amino acid sequence selected from the group consisting of WGGGSGASZS GAS; SESZNLYY; NDFZTISR; SGSZNLYY; and NGFZTISR, wherein Z comprises a region of variable amino acid composition and length. Optionally, a test polypeptide can include a sequence set forth in Table 1 or Table 2.

In some cases, a determination may be made, based on the results of a binding assessment process, to repeat one or more steps used to select the affinity reagents. For example, a method for characterizing affinity reagents can include the steps of (a) providing a library of affinity reagents; (b) performing an affinity reagent selection process, including: (i) contacting the library with solid-phase target analytes, thereby forming solid-phase complexes including the target analytes and a subset of the candidate affinity reagents, and (ii) separating the subset of candidate affinity reagents from the library, thereby obtaining a pool of selected affinity reagents; (c) performing binding assessment process, including: (i) contacting the selected affinity reagents with test analytes, thereby forming complexes including the test analytes and a subset of the selected affinity reagents, (ii) separating the subset of selected affinity reagents from the pool, thereby obtaining a plurality of candidate affinity reagents, and (iii) detecting candidate affinity reagents of the plurality of candidate affinity reagents; and (d) repeating step (b) using selected affinity reagents from the subset instead of the library of affinity reagents. Alternatively, one or more of the selected affinity reagents from the subset can be modified to produce a second library of affinity reagents and step (b) can be repeated using the second library of affinity reagents instead of the library of affinity reagents.

Affinity reagents that are obtained from a selection process set forth herein can be evaluated to determine their structure, function or both. Optionally, structure or function of affinity reagents can be determined after performing a binding assessment process set forth herein. For example, the binding of a plurality of different affinity reagents can be assessed as a pool in a binding assessment process, and then affinity reagents can be separated from the pool for individual evaluation of structural or functional characteristics. However, the binding assessment process set forth herein is optional and some or all steps of the process need not be performed. For example, different affinity reagents that have been obtained from a selection process can be individually evaluated with respect to structure or function absent performing a binding assessment on the pool from which the affinity reagents were derived. In some cases, some or all steps of a binding assessment process set forth herein can be performed after one or more affinity reagents have been individually evaluated for structure or function.

The structure or function of selected affinity reagent can be individually evaluated using methods known in the art. For example, the nucleic acid sequences of individual aptamers can be determined. Multiplex methods such as next generation sequencing can be used to determine sequences for a large number of aptamers in parallel. The number of affinity reagents that is evaluated for structure or function, for example, in a parallel process can include at least 2, 10, 100, 1×10³, 1×10⁴, 1×10⁶, 1×10⁸, 1×10¹⁰ or more different affinity reagents. Alternatively or additionally, the number can be at most 1×10¹⁰, 1×10⁸, 1×10⁶, 1×10⁴, 1×10³, 100, 10 or 2 different affinity reagents.

### Producing Probes Having Affinity Reagents

One or more affinity reagents can be attached to a particle to produce a probe. The probe can optionally include at least one label that is capable of producing a signal that can be detected to identify, quantify or characterize the probe. However, a probe of the present disclosure need not include a label. Exemplary probes having particles attached to one or more affinity reagents are set forth in US Pat. App. Pub. No. 2022/0162684 A1, which is incorporated herein by reference.

The present disclosure provides a method for producing probes. The method can include steps of (a) forming a first mixture including affinity reagents and particles, thereby producing probes, the probes each including a particle attached to an affinity reagent; and (b) separating the probes from affinity reagents and particles of the third mixture. The particles can be solid-phase particles having a composition set forth herein or known in the art. In some cases, the particles can be structured nucleic acid particles.

Particularly useful structured nucleic acid particles include nucleic acid origami. A nucleic acid origami can include one or more nucleic acids having tertiary or quaternary structures such as spheres, cages, tubules, boxes, triangles, icosahedrons, tiles, blocks, trees, pyramids, wheels or combinations thereof. Examples of such structures formed with DNA origami are set forth in Zhao et al. Nano Lett. 11, 2997-3002 (2011); Rothemund Nature 440:297-302 (2006); Sigle et al, Nature Materials 20:1281-1289 (2021); or U.S. Pat. No. 8,501,923 or 9,340,416, each of which is incorporated herein by reference. In some configurations, a nucleic acid origami may include a scaffold nucleic acid and a plurality of staple nucleic acids. The scaffold can be configured as a single, continuous strand of nucleic acid, and the staples can be formed by nucleic acids that hybridize, in whole or in part, with the scaffold nucleic acid. A particle including one or more nucleic acids (e.g. as found in origami or nanoball structures) may include regions of single-stranded nucleic acid, regions of double-stranded nucleic acid, or combinations thereof.

In some configurations, a nucleic acid origami includes a scaffold composed of a nucleic acid strand to which a plurality of oligonucleotides is hybridized. A nucleic acid origami may have a single scaffold molecule or multiple scaffold molecules. A scaffold nucleic acid can be linear (i.e. having a 3′ end and 5′ end) or circular (i.e. closed such that the scaffold lacks a 3′ end and 5′ end). A nucleic acid scaffold can be derived from a natural source, such as a viral genome or a bacterial plasmid. For example, a nucleic acid scaffold can include a single strand of an M13 viral genome. In other configurations, a nucleic acid scaffold may be synthetic, for example, having a non-naturally occurring sequence in full or in part. A scaffold nucleic acid can be single stranded but for a plurality of oligonucleotides hybridized thereto or short regions of internal complementarity. The size of a nucleic acid scaffold may vary to accommodate different uses. For example, a nucleic acid scaffold may include at least about 100, 500, 1000, 2500, 5000, 10000, 50000 or more nucleotides. Alternatively or additionally, a nucleic acid scaffold may include at most about 50000, 10000, 5000, 2500, 1000, 500, 100 or fewer nucleotides.

A nucleic acid origami can include a plurality of oligonucleotides that are hybridized to a scaffold nucleic acid. A first region of an oligonucleotide sequence can be hybridized to a scaffold nucleic acid while a second region is not hybridized to the scaffold. The second region can be in a single stranded state or, alternatively, can participate in a hairpin or other self-annealed structure in the oligonucleotide. In some cases, the second region of the oligonucleotide can hybridize to a complementary oligonucleotide to form a double-stranded region. An oligonucleotide can include two sequence regions that are hybridized to a scaffold nucleic acid, for example, to function as a ‘staple’ that restrains the structure of the scaffold. For example, a single oligonucleotide can hybridize to two regions of a scaffold that are separated from each other in the primary sequence of the scaffold. As such, the oligonucleotide can function to retain those two regions of the scaffold in proximity to each other or to otherwise constrain the scaffold to a desired conformation. Two sequence regions of an oligonucleotide staple can be adjacent to each other in the oligonucleotide sequence or separated by a third region that does not hybridize to the scaffold. One or more regions of an oligonucleotide that hybridize to a scaffold nucleic acid can be located at or near the 5′ end of the oligonucleotide, at or near the 3′ end of the oligonucleotide, or in a region of the oligonucleotide that is between the end regions. Oligonucleotides can be configured to hybridize with a nucleic acid scaffold, another oligonucleotide, a staple oligonucleotide, or a combination thereof. The oligonucleotides can be linear (i.e. having a 3′ end and a 5′ end) or closed (i.e. circular, lacking both 3′ and 5′ ends).

An oligonucleotide that is included in a nucleic acid origami can have any of a variety of lengths. An oligonucleotide may have a length of at least about 10, 25, 50, 100, 250, 500, or more nucleotides. Alternatively or additionally, an oligonucleotide may have a length of no more than about 500, 250, 100, 50, 25, 10, or fewer nucleotides. An oligonucleotide in a nucleic acid origami may hybridize with another oligonucleotide or a scaffold strand forming a particular number of base pairs. An oligonucleotide may form a hybridization region of at least about 5, 6, 7, 8, 9, 10, 15, 20, 25, 50 or more consecutive or total base pairs. Alternatively or additionally, an oligonucleotide may form a hybridization region of no more than about 50, 25, 20, 15, 10, 9, 8, 7, 6, 5, or fewer consecutive or total base pairs.

An affinity reagent, label, reactive moiety or other moiety can be attached to nucleic acid origami via a scaffold component or oligonucleotide component of the origami structure. For example, the scaffold or oligonucleotide can include a nucleotide analog that attaches covalently or non-covalently to an affinity reagent, label or other moiety. Examples of structured nucleic acid particles that include nucleic acid origami are set forth, for example, in U.S. Pat. No. 11,203,612; US Pat. App. Pub. No. 2022/0162684 A1 or U.S. patent application Ser. No. 17/692,035, each of which is incorporated herein by reference.

A structured nucleic acid particle (e.g., nucleic acid origami, or nucleic acid nanoball) may be formed by an appropriate technique including, for example, those known in the art. Nucleic acid origami can be designed, for example, as described in Rothemund, Nature 440:297-302 (2006), or U.S. Pat. No. 8,501,923 or 9,340,416, each of which is incorporated herein by reference. Nucleic acid origami may be designed using a software package, such as CADNANO (cadnano.org), ATHENA (github.com/lcbb/athena), or DAEDALUS (daedalus-dna-origami.org).

Another type of structured nucleic acid particle is a nucleic acid nanoball. Nucleic acid nanoballs may be fabricated by a method such as rolling circle amplification using a circular template to generate a nucleic acid amplicon consisting of a concatemer of template complements. The amplicon can be further modified to include crosslinks, for example, in the form of staples that hybridize to different regions of the amplicon. Exemplary methods for making nucleic acid nanoballs are described, for example, in U.S. Pat. No. 8,445,194, which is incorporated herein by reference.

A particle need not be composed primarily of nucleic acid and, in some cases, may be devoid of nucleic acids. For example, a particle can be composed of a solid support material, such as a silicon or silica nanoparticle, a carbon nanoparticle, a cellulose nanobead, a PEG nanobead, a polymeric nanoparticle (e.g., polyethyleneimine, dendritic polymer, dendrimer, polyacrylate particle, polystyrene-based particle, FluoSphere™, etc.), upconversion nanocrystal, or a quantum dot. A particle may include solid materials and shell-like materials (e.g., carbon nanospheres, silicon oxide nanoshells, iron oxide nanospheres, polymethylmethacrylate nanospheres, etc.). A particle may include distinct surfaces, such as plates or shells. In some configurations, a particle may include a gel material.

A particle may have any of a variety of sizes and shapes to accommodate use in a desired application. For example, a particle can have a regular or symmetric shape or, alternatively, a particle can have an irregular or asymmetric shape. The shape can be rigid or pliable. The size or shape of a particle can be characterized with respect to length, area, or volume. The length, area or volume can be characterized in terms of a minimum, maximum, or average for a population. Optionally, a particle can have a minimum, maximum or average length of at least about 50 nm, 100 nm, 250 nm, 500 nm, 1000 nm or more. Alternatively or additionally, a particle can have a minimum, maximum or average length of no more than about 1000 nm, 500 nm, 250 nm, 100 nm, 51 nm, or less. Optionally, a particle can have a minimum, maximum or average volume of at least about 1 μm³, 10 μm³, 100 μm³, 1 mm³, 10 mm³, 100 mm³, 1 cm³ or more. Alternatively or additionally, a particle can have a minimum, maximum or average volume of no more than about 1 cm³, 100 mm³, 10 mm³, 1 mm³, 100 μm³, 10 μm³, 1 μm³ or less.

A particle can be characterized with respect to its footprint (e.g. occupied area on a surface). The footprint may have a regular shape or an approximately regular shape, such as triangular, square, rectangular, circular, ovoid, or polygon. Optionally, the minimum, maximum or average area for a particle footprint can be at least about 10 nm², 100 nm², 1 μm², 10 μm², 100 μm², 1 mm² or more. Alternatively or additionally, the minimum, maximum or average area for a particle footprint can be at most about 1 mm², 100 μm², 10 μm², 1 μm², 100 nm², 10 nm², or less.

A particle that is made or used in a method set forth herein can be suspended in a fluid, immobilized on a solid support, or immobilized in another material such as a solid support material. For example, a population of particles can be colloidal for some, or all steps of a method set forth herein. Alternatively, a population of particles can be immobilized in, or on a solid support, for example, by gravity, non-covalent bonding, covalent bonding, coordination, adhesion or a combination thereof. Optionally, a first particle can be attached to a second particle. The first and second particles can compose the same material as each other or different materials from each other.

A method of the present disclosure can include steps for making structured nucleic acid particles having nucleic acid origami. For example, the method can include steps of (a) forming a mixture including a plurality of nucleic acid scaffolds and nucleic acid staples, thereby producing structured nucleic acid particles, the structured nucleic acid particles each including a nucleic acid scaffold and a plurality of nucleic acid staples folded into a nucleic acid origami; and (b) separating the structured nucleic acid particles from nucleic acid scaffolds and nucleic acid staples of the mixture.

A structured nucleic acid particle can be formed between a scaffold nucleic acid and a plurality of staple nucleic acids. The nucleic acids can be combined to form a mixture and the nucleic acids can be denatured, for example, due to elevated temperature or presence of chemical denaturants. The nucleic acids can spontaneously fold into a nucleic acid origami structure by reducing the temperature or removing chemical denaturants. For example, denaturation of nucleic acids can occur at temperatures above 60° C., 70° C., 80° C., 90° C. or higher including for example, up to the boiling point for the fluid in which denaturation is carried out. Origami can be produced by reducing the temperature to 40° C., 35° C., 30° C., 25° C., 20° C. or lower. Temperature reduction can occur at a predefined rate, for example, using a thermostat or other temperature regulating device. The rate at which temperature is reduced can be at least 1° C./5 min, 1° C./3 min, 1° C./2 min, 1° C./1 min or faster. Alternatively or additionally, the rate at which temperature is reduced can be at least 1° C./1 min, 1° C./2 min, 1° C./3 min, 1° C./5 min or slower. It will be understood that temperature reduction need not be regulated and can instead occur due to spontaneous cooling.

A mixture of nucleic acids can be contained in a vessel that is heated or cooled via conduction, convection or radiation. For example, the vessel or contents of the vessel can be heated or cooled by conduction from a solid block such as a solid block that is coupled to a Peltier device. Useful apparatus include PCR machines and other laboratory heating devices. Convection can occur between a fluid such as a gas or liquid that is in contact with the vessel or contents of the vessel. For example, the fluid can be coupled to a heating or cooling element such as those found in an oven and water bath. A programmable temperature water bath, for example, commercially available for laboratory use or kitchen use (e.g. Sous-vide cooker) can be useful. A convection oven or refrigerator can also be useful for heating or cooling a mixture of nucleic acids in a method set forth herein. A vessel or contents of the vessel can be contacted with electromagnetic radiation, for example, in the microwave, infrared or visible regions of the spectrum.

A mixture of nucleic acids that is used to produce nucleic acid origami can include magnesium (e.g. MgCl₂), pH buffer, water, salt or other reaction components. Exemplary components are set forth in Rothemund, Nature 440:297-302 (2006); U.S. Pat. Nos. 8,501,923, 9,340,416 or 11,203,612; US Pat. App. Pub. No. 2022/0162684 A1; or U.S. patent application Ser. No. 17/692,035, each of which is incorporated herein by reference. See also Example VII hereinbelow.

Once nucleic acid origami structures have been formed by binding between scaffolds and staples, the origami structures can be separated from unbound nucleic acid scaffolds and unbound nucleic acid staples. Unbound scaffolds and staples can be separated from origami using chromatography such as reverse phase chromatography, size exclusion chromatography, ion exchange chromatography, affinity chromatography or the like. Unbound scaffolds and staples can be separated from origami using dialysis. Dialysis membranes can be selected to have a molecular weight cutoff that allows unbound scaffolds and/or staples to pass through the membrane while origami structures are retained. Dialysis membranes can be made from regenerated cellulose or cellulose esters.

Separation can be achieved by filtration using molecular weight cutoff filters that pass scaffolds, staples and other relatively small mixture components into the filtrate while the retentate contains the origami structures. Fluids can be passed through the filter using positive displacement (e.g. from a pump), negative displacement (e.g. from a vacuum), application of a concentration gradient across a filter, application of centrifugal force across the filter or the like. Useful apparatus include, but are not limited to stirred filter devices, such as an Amicon™ stirred cell. Tangential flow filtration devices are also useful. Useful filter materials include, for example, regenerated cellulose or polyethersulfone.

A filter or dialysis membrane used for separation of structured nucleic acid particles from other components used to make or modify the structured nucleic acid particles can have a molecular weight cutoff of at least about 10 kDa, 50 kDa, 100 kDa, 500 kDa or 1000 kDa. Alternatively or additionally, the molecular weight cutoff for a filter or dialysis membrane can be at most about 1000 kDa, 500 kDa, 100 kDa, 50 kDa or 10 kDa.

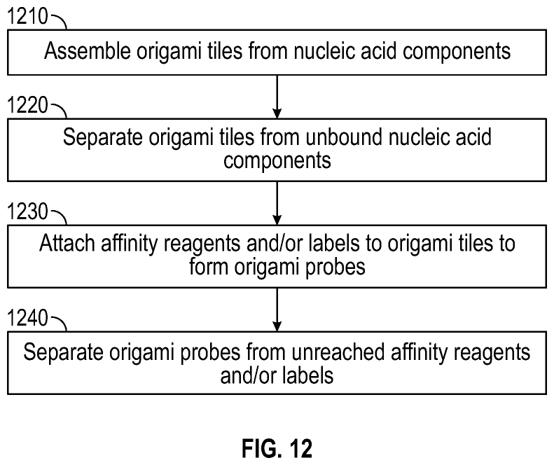

Optionally, structured nucleic acid particles having nucleic acid origami can be attached to affinity reagents to form probes. As diagrammed in FIG. 12, a method of producing probes can start at step 1210 with the assembly of origami tiles from nucleic acid components such as staples and scaffolds. At step 1220 the origami tiles can be separated from unbound components of the assembly mixture. Proceeding to step 1230, affinity reagents and/or labels can be attached to the origami tiles to form probes. For example, affinity reagents or labels can include a nucleic acid component having a nucleotide sequence that hybridizes to a complementary sequence in an origami tile. At step 1240, the probes can be separated from one or more unreacted components of the attachment process such as origami tiles, affinity reagents or labels. The steps in FIG. 12 are optional. One or more of the steps can be omitted, repeated or performed in a different order than exemplified in the figure. Moreover, one or more steps of the process can be carried out for structured nucleic acid particles other than origami tiles.

In some configurations, a method of producing probes can include steps of (a) forming a first mixture including a plurality of nucleic acid scaffolds and nucleic acid staples, thereby producing structured nucleic acid particles, the structured nucleic acid particles each including a nucleic acid scaffold and a plurality of nucleic acid staples folded into a nucleic acid origami; (b) separating the structured nucleic acid particles from nucleic acid scaffolds and nucleic acid staples of the first mixture; (c) forming a second mixture including a plurality of functionalized nucleic acids and a plurality of the structured nucleic acid particles, wherein the functionalized nucleic acids include affinity reagents, thereby producing probes, the probes each including a structured nucleic acid particle attached to an affinity reagent via the functionalized oligonucleotide; and (d) separating the probes from functionalized nucleic acids and structured nucleic acid particles of the second mixture.

Functionalized nucleic acids can include any of a variety of affinity reagents including, for example, those set forth herein or those known in the art. A particularly useful affinity reagent is a nucleic acid aptamer. A functionalized nucleic acid can include a sequence region that functions as an aptamer. The functionalized nucleic acid can also include a region that facilitates attachment of the aptamer to a structured nucleic acid particle such as a nucleic acid origami. The attachment region can be a single nucleotide, for example, a nucleotide having a reactive moiety that covalently bonds to a moiety on a particle, or binding moiety that binds non-covalently to its binding partner on a particle. The attachment region can be a nucleotide sequence that is complementary to a sequence on a structured nucleic acid particle such as a staple or scaffold component of a nucleic acid origami. The functionalized nucleic acid can attach to the structured nucleic acid via hybridization of the complementary sequences. Thus, a functionalized nucleic acid can have a nucleotide sequence that includes an aptamer sequence region and an attachment region. However, an aptamer sequence of a functionalized nucleic acid need not be contiguous with an attachment region. For example, the aptamer sequence can be linked to the attachment region via a non-nucleic acid linker moiety.

A functionalized nucleic acid can be attached to a non-nucleic acid affinity reagent including, for example, an antibody, such as a full-length antibody or functional fragment thereof. A functional fragment of an antibody can include, for example, a Fab, Fab′, F(ab′)₂, single-chain variable (scFv), di-scFv, tri-scFv, or microantibody. Other affinity reagents that can be attached to a functionalized nucleic acid include, for example, affibodies, affilins, affimers, affitins, alphabodies, anticalins, avimers, DARPins, monobodies, nanoCLAMPs, protein aptamers, lectins or functional fragments thereof. Functionalized nucleic acids that are attached to non-nucleic acid affinity reagents can be attached to a structured nucleic acid particle, such as an origami, as set forth herein for aptamers. However, it will be understood that affinity reagents need not be attached to a particle via a functionalized nucleic acid. For example, affinity reagents can be attached using covalent or non-covalent chemistries.

A functionalized nucleic acid can be attached to moieties other than affinity reagents. For example, a functionalized nucleic acid can be attached to a label. The label can be any molecule or moiety that provides a detectable characteristic. The detectable characteristic can be, for example, an optical signal such as absorbance of radiation, luminescence emission, luminescence lifetime, luminescence polarization, fluorescence emission, fluorescence lifetime, fluorescence polarization, or the like; Rayleigh and/or Mie scattering; binding affinity for a ligand or receptor; magnetic properties; electrical properties; charge; mass; radioactivity or the like. Exemplary labels include, without limitation, a luminophore (e.g. fluorophore), chromophore, nanoparticle (e.g., gold, silver, carbon nanotubes, quantum dots, upconversion nanocrystals), heavy atoms, radioactive isotope, mass label, charge label, spin label, receptor, ligand, or the like. A label may produce a signal that is detectable in real-time (e.g., fluorescence, luminescence, radioactivity). A label may produce a signal that is detected off-line (e.g., a nucleic acid barcode) or in a time-resolved manner (e.g., time-resolved fluorescence). A label may produce a signal with a characteristic frequency, intensity, polarity, duration, wavelength, sequence, or fingerprint. Any of a variety of moieties other than affinity reagents or labels can be attached to a functionalized nucleic acid or structured nucleic acid particle.

Useful chemistries for attaching functionalized nucleic acids, affinity reagents, labels or other moieties to structured nucleic acid particles, such as nucleic acid origami, include any of a variety of those set forth herein or known in the art. For example, click chemistry and other chemistries can be used as set forth herein in the Examples or in the context of attaching target analytes to solid-phase supports, particles or other objects. Other useful chemistries are set forth in U.S. Pat. No. 11,203,612 and US Pat. App. Pub. No. 2022/0162684 A1, each of which is incorporated herein by reference.

A particle can be attached to one or more functionalized nucleic acid using a method set forth herein. Structured nucleic acid particles, such as nucleic acid origami, can be engineered to contain one or more attachment moieties at specified locations in the structure. For example, a nucleic acid origami can include one or more staples each having a nucleotide sequence that functions as an attachment moiety to which a functionalized nucleic acid can hybridize. Similarly, a nucleic acid origami can be assembled to include one or more staples each having a reactive moiety or binding moiety that is capable of attaching to a functionalized nucleic acid or other moiety (e.g. affinity reagent or label). Accordingly, a structured nucleic acid particle, such as a nucleic acid origami, can include at least 1, 2, 3, 4, 6, 8, 10, 15, 20, 25 or more attachment moieties. Alternatively or additionally, a structured nucleic acid particle such as a nucleic acid origami can include at most 25, 20, 15, 10, 8, 6, 5, 4, 3, 2, or 1 attachment moieties. The structured nucleic acid particles can include a quantity of affinity reagents, labels or other moieties in these ranges as well. The moieties can be located at engineered locations on a structured nucleic acid particle. For example, moieties of one type (e.g. affinity reagents) can be located on one face of an origami tile while moieties of another type (e.g. labels) are located on another face of the tile. Numbers and locations of moieties on structured nucleic acid particles can optionally be configured as set forth in US Pat. App. Pub. No. 2022/0162684 A1, which is incorporated herein by reference.

Once probes have been formed, they can be separated from structured nucleic acid particles and/or affinity reagents that were not incorporated into probes. Separation can employ chromatography, dialysis, filtration or other techniques set forth herein in the context of separating nucleic acid origami from excess nucleic acid components used to produce the origami. For example, separation can be achieved using a filter or dialysis membrane having a molecular weight cutoff that allows passage of affinity reagents and/or structured nucleic acid particles while retaining the probes. Separation can also be achieved, for example, by precipitation of affinity reagents using conditions wherein the probes remain in solution. For example, antibodies and other protein-based affinity reagents can be precipitated by salting out (e.g. using ammonium sulfate), by non-ionic hydrophilic polymers (e.g. dextrans or polyethylene glycols), or other reagents that can preferentially precipitate proteins rather than nucleic acids. Alternatively, probes can be selectively precipitated using conditions wherein affinity reagents are soluble. For example, structured nucleic acids can be precipitated by ethanol or isopropanol while retaining protein-based affinity reagents in solution. Other separation techniques include, for example, liquid-liquid extraction or solid-phase extraction.

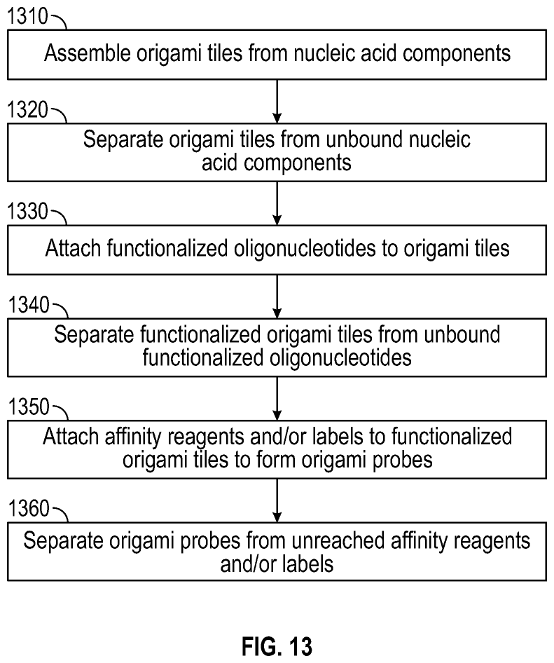

Probes can be formed by attachment of affinity reagents to structured nucleic acid particles as diagrammed in FIG. 13. At step 1310, structured nucleic acid particles, such as origami tiles, are assembled from nucleic acid components such as staples and scaffolds. At step 1320 the origami tiles can be separated from unreacted components of the assembly mixture. Proceeding to step 1330, functionalized nucleic acids are attached to origami tiles to form functionalized origami tiles. For example, functionalized nucleic acids can include nucleotide sequences that hybridize to sequences in a scaffold or staple component of an origami tile to form functionalized origami tiles. At step 1340, the functionalized origami tiles are separated from origami tiles and/or functionalized nucleic acids that did not react to form the origami tiles. Proceeding to step 1350, affinity reagents and/or labels can be attached to functionalized origami tiles to form probes. At step 1360, the probes can be separated from one or more unreacted components of the attachment process such as origami tiles, affinity reagents or labels. The steps in FIG. 13 are optional. One or more of the steps can be omitted, repeated or performed in a different order than exemplified in the figure. Moreover, one or more steps of the process can be carried out for structured nucleic acid particles other than origami tiles.

In some configurations, a method of producing probes can include steps of (a) forming a first mixture including a plurality of nucleic acid scaffolds and nucleic acid staples, thereby producing structured nucleic acid particles, the structured nucleic acid particles each including a nucleic acid scaffold and a plurality of nucleic acid staples folded into a nucleic acid origami; (b) separating the structured nucleic acid particles from nucleic acid scaffolds and nucleic acid staples of the first mixture; (c) forming a second mixture including a plurality of functionalized nucleic acids and a plurality of the structured nucleic acid particles, thereby producing functionalized structured nucleic acid particles, the functionalized structured nucleic acid particles each including a nucleic acid origami hybridized to a functionalized nucleic acid; (d) separating the functionalized structured nucleic acid particles from functionalized nucleic acids and structured nucleic acid particles of the second mixture; (e) forming a third mixture including affinity reagents and the functionalized structured nucleic acid particles, thereby producing probes, the probes each including a structured nucleic acid particle attached to an affinity reagent via a moiety of the functionalized oligonucleotide; and (f) separating the probes from affinity reagents and functionalized structured nucleic acid particles of the third mixture.

Affinity reagents can be attached to functionalized structured nucleic acid particles using methods and compositions set forth herein or known in the art. Functionalized structured nucleic acid particles can be separated from structured nucleic acid particles and/or affinity reagents that were not incorporated into functionalized structured nucleic acid particles using separation techniques set forth herein or known in the art.

### Example I

### Selection of DNA Aptamers with High Throughput Microbead SELEX

### Immobilization of Peptide Targets

CNBr-activated Sepharose 4B beads having a size range between 45-165 microns are suspended in 1 mM HCl, and after 30 minutes are separated using a nylon sieve (e.g. 50, 60, 70, 85, 100 or 150 micron mesh size) to select beads having a size greater than or equal to the mesh size selected. Beads are washed with 1 mM HCl and collected.

Peptide targets are dissolved in coupling buffer (0.1 M NaHCO₃ pH 8.3, 0.5 M NaCl) to a final peptide concentration 20, 30, 40, 50, 60 or 100 mM in 300, 400, 500, 600 or 700 μL of coupling buffer, mixed with 10, 25, 50 or 75 μL of washed beads and incubated overnight at 4° C. with constant mixing. Beads are washed with 2×4 mL of coupling buffer to remove unreacted peptides. Unreacted active groups on the beads are blocked by addition of 0.1 M Tris-HCl buffer, pH 8.0 (4 mL) and incubation for at least 2 hours at room temperature with constant mixing. Beads are then washed with at least three cycles of alternating pH, each cycle including a 4 mL wash with 0.1 M acetic acid/sodium acetate, pH 4.0 containing 0.5 M NaCl followed by a wash with 4 mL 0.1 M Tris-HCl, pH 8 containing 0.5 M NaCl.

### Microbead SELEX Procedure

Samples are prepared as follows. For the first cycle, 80 to 100 μL AS buffer, 2 to 10 μL 2× concentrated AS buffer and 2-10 μL 100 μM aptamers library was added to a PCR tube. AS buffer is selected from AS-1 buffer (136 mM NaCl, 12 mM KCl, 10 mM Na₂HPO₄, 1.76 mM KH₂PO₄, 4.9 mM MgCl₂, 0.01% Tween 20, pH 7.5); AS-2 buffer (150 mM NaCl, 20 mM KCl, 15 mM Na₂HPO₄, 2.00 mM KH₂PO₄, 3.5 mM MgCl₂, 0.01% Tween 20, pH 7.4); AS-3 buffer (20 mM Tris-HCL (pH7.4), 2.5 mM MgCl₂, 2.5 mM CaCl₂, 120 mM NaCl, 2.25 mM KCl, 0.05% Tween-20); AS-4 buffer (10 mM HEPES, 120 mM NaCl, 5 mM MgCl₂, 5 mM KCl, pH 7.4); AS-5 buffer (25 mM MES (pH 6.0), 2.5 mM MgCl₂, 0.5 mM CaCl₂, 10 mM NaCl, 2.25 mM KCl, 0.005% Tween-20); AS-6 buffer (20 mM Tris-HCl (ph7.4), 5 mM MgCl₂, 5 mM CaCl₂, 60 mM NaCl, 2.25 mM KCl, 0.05% Tween-20); AS-7 buffer (50 mM tris-HCl, 136 mM NaCl, 4.9 mM MgCl₂, 4.9 mM CaCl₂, 0.01% Tween-20); AS-8 buffer (50 mM tris-HCl, 136 mM NaCl, 4.9 mM MgCl₂, 4.9 mM CaCl₂, 0.01% Tween-20); or AS-9 buffer (50 mM tris-HCl, 136 mM NaCl, 9.8 mM MgCl₂, 0.01% Tween-20). For the second and third cycles, 4, 6, 8 or 10 pmols of aptamer sample obtained after the first and second cycle, respectively, in AS buffer is added to a PCR tube. For the fourth and subsequent cycles, 2, 4, 6 or 8 pmols of aptamer sample obtained after the previous cycle in 100 μL of AS buffer is added to a PCR tube.

AS buffer (200 μL) is added to each well of a U-bottom plate. Then agarose beads (from 40-200 beads for the first cycle), or 20 to 120 agarose beads (for subsequent cycles), coated with target peptide is added to each well for inspection. The bead slurry is then transferred from each well of the U-bottom plate to a well of a 96-well filter plate (e.g. 96-well spin column with 5, 20 or 40 μm frit) and centrifuged for 30 sec at 1000×g. Following centrifugation, 200 μL of AS buffer is added to each well of the filter plate for 5 min incubation at room temperature followed by centrifugation.

Aptamer library products are transferred from a PCR plate to each well of the filter plate, mixed to dislodge the beads from the filter membrane, and then the bead slurries are transferred back to the PCR plate. The PCR plate is sealed and incubated for 1-16 hours with continuous shaking.

The agarose beads are washed two to four times in each of three separate filter plates for a total of six to twelve washes per bead sample. Agarose beads are transferred to a first filter plate and centrifuged at 1000×g for 20 sec at room temperature. Then 200 μL of AS buffer is added to each well and again centrifuged. The AS buffer wash and centrifugation can be repeated. As such, beads can be washed one to four times in the same well of the filter plate. The agarose beads from each well of the first filter plate are then resuspended in 200 μL of AS buffer and moved to a well of a second filter plate where the beads are washed with two to four rounds of AS buffer (200 μL) and centrifugation. The agarose beads from each well of the second filter plate are then resuspended in 200 μL of AS buffer and moved to a well of a third filter plate, where the beads are washed with two to four rounds of AS buffer (200 μL) and centrifugation. As such, each set of beads is washed two to four times in three filter plates.

### PCR

Aptamer libraries produced by the above SELEX process are amplified by PCR as follows. To each well of the third filter plate is added 50 μL of Taq PCR mix (2.5 to 5 mM MgCl₂, 0.5 to 1 μM forward primer, 0.5 to 1 μM reverse primer, and 0.5 mM dNTPs and 2 to 2.5 units of Taq polymerase). The beads are suspended and the resulting bead slurry transferred to a well of a PCR plate. PCR is performed for 24 to 28 cycles. Initial denaturation is performed for 3 min. at 98° C. Each preparative PCR cycle includes 15 sec. at 98° C., 30 sec. at 65° C. and 30 sec. at 72° C. Obtained DNA is reamplified by addition of 2 or 5 ul of post-PCR mix into 2×100 μL of KAPA PCR mix (0.5 to 1 μM forward primer, 0.5 to 1 μM reverse primer, 1×SYBR Green in 1×KAPA PCR mix). PCR is carried out on a qPCR thermocycler until an amplification plateau is reached. The PCR starts with 3 min. at 98° C., then each cycle includes 15 sec. at 98° C., 2 sec. at 65° C. and 6 sec. at 72° C. After PCR, DNA is purified with Monarch PCR & DNA Clean up kit (New England BioLabs) or NucleoSpin Gel and PCR clean-up (Macherey-Nagel).

Amplification and Reamplification of FB Libraries DNA-EdU for Microbead SELEX with Modified Nucleotides.

Aptamer libraries used in the SELEX process are amplified by PCR as follows. To each well of the third filter plate is added 50 μL of PCR mix (1×Pwo reaction buffer with 2 mM MgSO₄, 0.5 to 1 mM FB forward primer, 0.5 to 1 mM FB reverse primer, 250 mM dNTPs mix (dATP, dCTP, dGTP and 5-Ethynyl-dUTP), 2.5 U Pwo polymerase and 1×SYBR Green). The beads are suspended and the resulting bead slurry transferred to a well of a PCR plate.

PCR is performed on a qPCR thermocycler until plateau of reaction is reached. Initial denaturation is performed in 95° C. for 2 min. Each PCR cycle includes 30 sec. at 95° C., 20 sec. at 62.5 or 65° C. and 20 sec. at 72° C.

Obtained DNA-EdU is reamplified by addition of 2 or 5 ul of post-PCR mix into 3, 4, 5 or 6×100 ml of Pwo PCR mix. PCR is performed under the same conditions as presented above. After PCR, DNA is purified with Monarch PCR & DNA Clean up kit (New England BioLabs) or NucleoSpin Gel and PCR clean-up (Macherey-Nagel).

Regeneration of ssDNA from dsDNA (Natural and Modified Nucleotides)

Double stranded DNA (dsDNA) is digested with 100 U of exonuclease lambda in 1× lambda exonuclease lambda buffer in 250 μl total reaction volume. Reactions are carried out at 37° C. until all dsDNA is converted to single stranded DNA (ssDNA). The progress of the reaction is monitored by electrophoresis on a 4% agarose gel where the undigested and digested samples are compared. Single stranded DNA is purified from lambda exonuclease mix with Monarch PCR & DNA Clean up kit (New England BioLabs) or NucleoSpin Gel and PCR clean-up (Macherey-Nagel) and eluted with dH₂O.

Library Functionalization for Microbead SELEX with Modified Nucleotides

In order to modify ssDNA-EdU, the following are mixed: 70 μl of ssDNA-EdU, 10 μl of 10 mM azide of choice (e.g., benzyl-azide), 10 μl of 100 mM phosphate buffer pH 7.0 and 10 μl of CuAAC solution. CuAAC mixture is prepared by mixing: 70 μl of dH₂O, 4 μl of 100 mM THPTA, 1 μl 100 mM CuSO₄ and 25 μl of freshly prepared 100 mM sodium ascorbate. Solution is incubated at room temperature for 10 minutes before addition to ssDNA. Library functionalization is carried out at 37° C., with constant mixing (800 r.p.m) for 1 hour. Immediately after reaction is finished, functionalized ssDNA is purified with Monarch PCR & DNA Clean up kit (New England BioLabs) or NucleoSpin Gel and PCR clean-up (Macherey-Nagel) and eluted with dH₂O. Maximum ssDNA-EdU capacity for single functionalization reaction is 500 pmols. Pfeiffer et al., Nature Protocols 13:1153-1180 (2018), which is incorporated herein by reference, provides a useful protocol for functionalizing aptamers with modified nucleotides.

### Example II

### Polypeptide-Based Molecular Constructs Having Structural Motifs for Improving Aptagenicity

This example describes polypeptide-based molecular constructs that are designed to present target epitope sequences for binding to aptamers. The constructs include target epitope sequences located between flanking sequences. The flanking sequences form structural motifs that are designed to provide high accessibility of the target epitope sequences for binding to aptamers. The polypeptide-based molecular constructs are expected to be useful as bait for obtaining aptamers in a SELEX process. The flanking sequences are also designed to have minimal interactions with aptamers during binding, thereby improving selection of aptamers that are specific for the target epitope sequences independent of sequence context.

Amino acid sequences are denoted herein using the IUPAC single letter code. In the sequences, “Z” represents a region of variable amino acid composition and length (e.g. 1, 2, 3, 4, 5, 6, or more naturally occurring and/or non-naturally occurring amino acids), and “X” represents a single amino acid of variable composition (e.g. naturally occurring or non-naturally occurring amino acids). One or more amino acids in Z or X can include or exclude a moiety that is identical or analogous to a post-translational modification.

Polypeptide bait molecules were designed to include a structural motif with a “corner” architecture. This structural motif has been identified from a comparison of three-dimensional structures for DNA binding proteins despite a high variability in the amino acid sequence compositions for the motif. See Wu et al. Nucl. Acids Res. 38:14 (2010), which is incorporated herein by reference. Exemplary amino acid sequences found to fold into a corner architecture include the sequence SESSKGNLYY found in the DNA binding pocket of the Staphylococcus aureus multidrug-binding protein QacR and NDFSQTTISR found in the POU domain of class 2 transcription factor I (Wu et al. supra). Polypeptide bait molecules were designed to each include an epitope region, amino flank and carboxyl flank. The term “amino flank” is used to indicate that the flank is attached to the amino terminus of the epitope region. The amino flank can be at the amino terminus of the polypeptide bait molecule, or internal to the polypeptide bait molecule. The term “carboxyl flank” is used to indicate that the flank is attached to the carboxyl terminus of the epitope region. The carboxyl flank can be at the carboxyl terminus of the polypeptide bait molecule, or internal to the polypeptide bait molecule.

A first set of polypeptide bait molecules was designed to have the sequence SESZNLYY. Versions having a single trimer epitope in the epitope region had sequence SESXXXNLYY. Versions in which Z was a single tetramer epitope, single pentamer epitope, single hexamer epitope or longer single epitope were contemplated. Versions having multiple epitopes in the epitope region were designed. For example, versions having two trimer epitopes in the epitope region had sequence SESWFRNLYYSGASXXXNLYY (SEQ ID NO: 53), wherein WFR is a first trimer epitope and XXX is a second timer epitope.

A second set of polypeptide bait molecules was designed to have the sequence NDFZTISR (SEQ ID NO: 54). Versions having a single trimer epitope in the epitope region had sequence NDFXXXTISR. Versions in which Z was a single tetramer epitope, single pentamer epitope, single hexamer epitope or longer single epitope were contemplated. Versions having multiple epitopes in the epitope region were designed. For example, versions having two trimer epitopes in the epitope region had sequence NDFWFRNLYYSGASXXXTISR (SEQ ID NO: 54), wherein WFR is a first trimer epitope and XXX is a second timer epitope.

A third set of polypeptide bait molecules was designed to have a modification of the amino flank of the Staphylococcus aureus multidrug-binding protein QacR corner motif. The sequence was SGSZNLYY, wherein the G residue replaced the E residue. The replacement of the charged E residue with the smaller and neutral G residue is expected to improve SELEX performance. Versions having a single trimer epitope in the epitope region had sequence SGSXXXNLYY. Versions in which Z was a single tetramer epitope, single pentamer epitope, single hexamer epitope or longer single epitope were contemplated. Versions having multiple epitopes in the epitope region were designed. For example, versions having two trimer epitopes in the epitope region had sequence SGSWFRNLYYSGASXXXNLYY (SEQ ID NO: 55), wherein WFR is a first trimer epitope and XXX is a second timer epitope.

A fourth set of polypeptide bait molecules was designed to have a modification of the amino flank of the class 2 transcription factor I corner motif. The sequence was NGFZTISR, wherein the G residue replaced the D residue. The replacement of the charged D residue with the smaller and neutral G residue is expected to improve SELEX performance. Versions having a single trimer epitope in the epitope region had sequence NGFXXXTISR. Versions in which Z was a single tetramer epitope, single pentamer epitope, single hexamer epitope or longer single epitope were contemplated. Versions having multiple epitopes in the epitope region were designed. For example, versions having two trimer epitopes in the epitope region had sequence NGFWFRNLYYSGASXXXTISR (SEQ ID NO: 56), wherein WFR is a first trimer epitope and XXX is a second timer epitope.

Table 1 provides a listing of amino acid sequences that can be included in polypeptide-based bait molecules.

TABLE 1

| | SEQ |
|---|---|
| Amino Acid | ID |
| Sequence | NO |
| WGGGSGASZSGAS | 3 |
| WGGGSGASXXXSGAS | 4 |
| SESSKGNLYY | 5 |
| SESZNLYY | 6 |
| SESXXXNLYY | 7 |
| SESZSGASZNLYY | 8 |
| SESWFRSGASZNLYY | 9 |
| SESWFRSGASXXXNLYY | 10 |
| NDFSQTTISR | 11 |
| NDFZTISR | 12 |
| NDFXXXTISR | 13 |
| NDFZSGASZTISR | 14 |
| NDFWFRSGASZTISR | 15 |
| NDFWFRSGASXXXTISR | 16 |
| SGSZNLYY | 17 |
| SGSXXXNLYY | 18 |
| SGSZSGASZNLYY | 19 |
| SGSWFRSGASZNLYY | 20 |
| SGSWFRSGASXXXNLYY | 21 |
| NGFZTISR | 22 |
| NGFXXXTISR | 23 |
| NGFZSGASZTISR | 24 |
| NGFWFRSGASZTISR | 25 |
| NGFWFRSGASXXXTISR | 26 |

In Table 1, “Z” represents a region of variable amino acid composition and length (e.g. 1, 2, 3, 4, 5, 6, or more naturally occurring and/or non-naturally occurring amino acids), and “X” represents a single amino acid of variable composition (e.g. naturally occurring or non-naturally occurring amino acids).

### Example III

### Using Structural Motifs as Epitope Target Flanks to Improve Aptagenicity

DNA aptamers were selected as set forth in Example I with the following modifications. Bait molecules were synthesized to include one of three trimer epitopes: RFF, WFR and GGG. The GGG epitope provides a negative control since it has been found to be non-aptagenic under the conditions used. The names and amino acid sequences for the bait molecules are shown in Table II. Amino acid sequences use the single letter amino acid code as defined by IUPAC.

TABLE 2

| | | SEQ |
|---|---|---|
| Name | Sequence | ID NO |
| RFF/SGAS | WGGGSGAS-RFF-SGAS | 27 |
| RFF/NDF | WGGGNDF-RFF-SGAS | 28 |
| RFF/NGF | WGGGNGF-RFF-SGAS | 29 |
| RFF/SES | WGGGSES-RFF-SGAS | 30 |
| RFF/SGS | WGGGSGS-RFF-SGAS | 31 |
| WFR/SGAS | WGGGSGAS-WFR-SGAS | 32 |
| WFR/NDF | WGGGNDF-WFR-SGAS | 33 |
| WFR/NGF | WGGGNGF-WFR-SGAS | 34 |
| WFR/SES | WGGGSES-WFR-SGAS | 35 |
| WFR/SGS | WGGGSGS-WFR-SGAS | 36 |
| GGG/SGAS | WGGGSGAS-GGG-SGAS | 37 |
| GGG/NDF | WGGGNDF-GGG-SGAS | 38 |
| GGG/NGF | WGGGNGF-GGG-SGAS | 39 |
| GGG/SES | WGGGSES-GGG-SGAS | 40 |
| GGG/SGS | WGGGSGS-GGG-SGAS | 41 |

The bait peptides listed in Table 2 were attached to beads as set forth in Example I. The bait peptides were screened against the following aptamer samples using the microbead SELEX procedure of Example I: 100 ng of CR3 naïve library (“naïve library”); a mixture of 100 ng of CR3 naïve library and 1 ng of aptamer previously selected against RFF (“7660”); or a mixture of 100 ng of CR3 naïve library and 1 ng of aptamer previously selected against WFR (“7527”). Beads were collected from each well, and 15 beads from each well were subjected to qPCR as set forth in Example I.

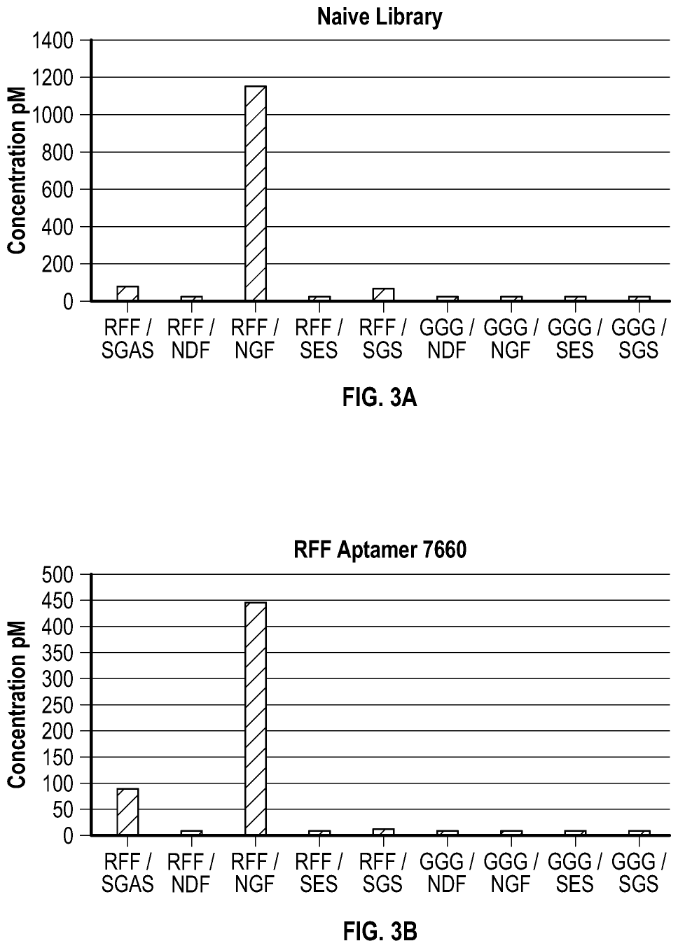

FIG. 3A shows quantities of aptamers selected from the naïve library using various bait molecules having the RFF epitope or control GGG epitope. FIG. 3B shows quantities of aptamers selected from the 7660 mixture using various bait molecules having the RFF epitope or control GGG epitope. The results indicated that both Aptamer and naïve library binding to the RFF trimer epitope was improved using the NGF amino flank and TISR (SEQ ID NO: 57) carboxyl flank.

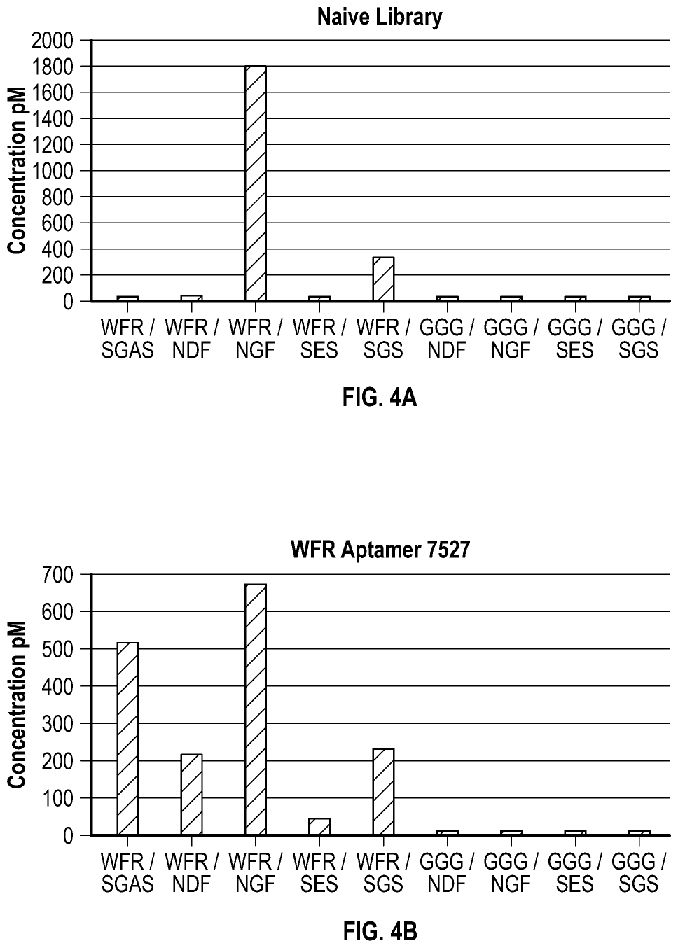

FIG. 4A shows quantities of aptamers selected from the naïve library using various bait molecules having the WFR epitope or control GGG epitope. FIG. 4B shows quantities of aptamers selected from the 7527 mixture using various bait molecules having the WFR epitope or control GGG epitope. The results indicated that both aptamer and naïve library binding to the WFR trimer epitope was improved using the NGF amino flank and TISR carboxyl flank.

### Example IV

### Three-Dimensional Structures for Target Polypeptides

This example provides polypeptides that are designed to present target epitope sequences for binding to affinity reagents such as aptamers.

Polypeptide structures were generated using PyMOL by modifying the polypeptide sequences that form the representative corner motif 3D structure published in Wu et al. Nucl. Acids Res. 38:14 (2010).

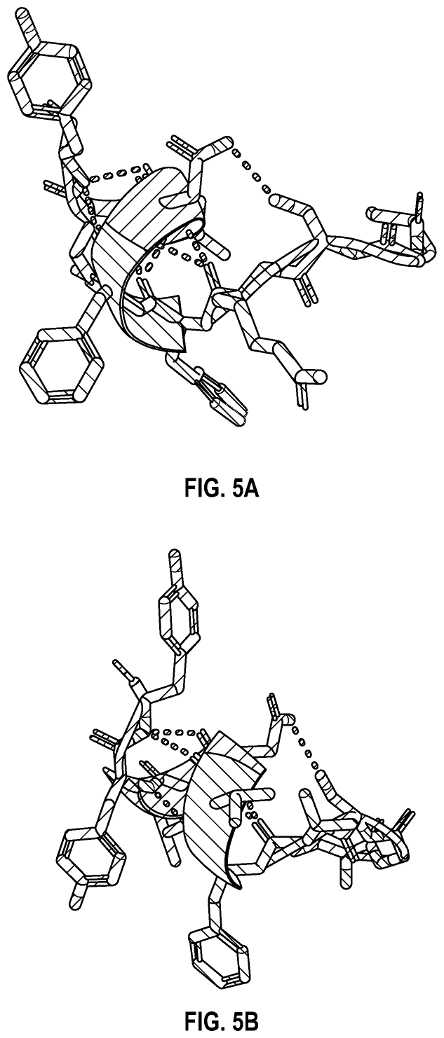

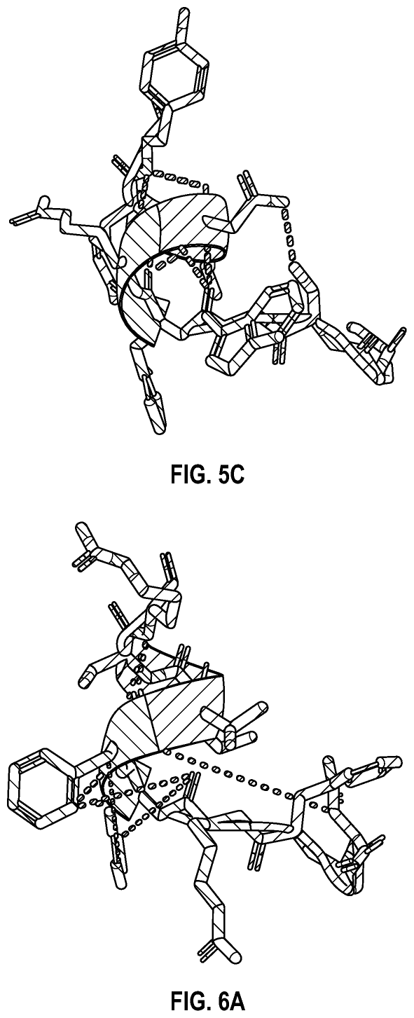

FIG. 5 shows computed three-dimensional models for polypeptides having the general structure SGSXXXNLY, wherein the amino flank includes the sequence SGS, the carboxy flank includes the sequence NLY and XXX is an epitope sequence. FIG. 5A shows the three-dimensional structure for the polypeptide SGSRFFNLYY, having epitope sequence RFF. FIG. 5B shows the three-dimensional structure for the polypeptide SGSTFTNLYY, having epitope sequence TFT. FIG. 5C shows the three-dimensional structure for the polypeptide SGSWFRNLYY, having epitope sequence WFR.

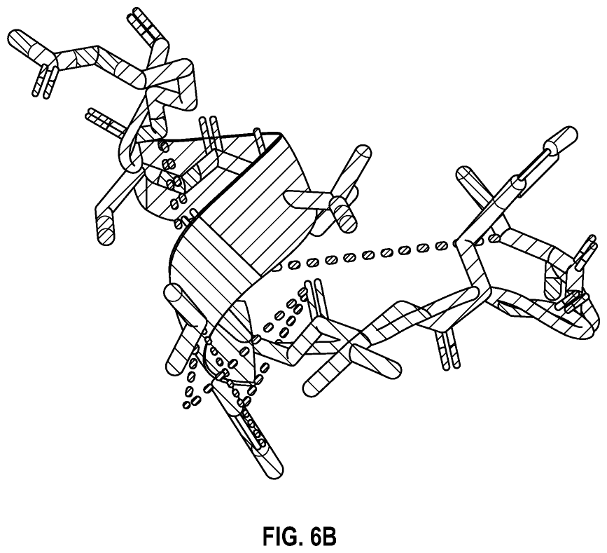

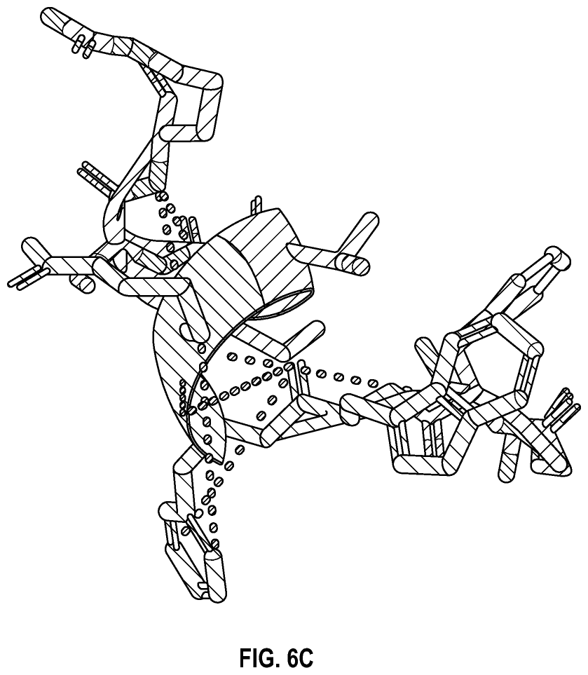

FIG. 6 shows computed three-dimensional models for polypeptides having the general structure NGFXXXTISR, wherein the amino flank includes the sequence NGF, the carboxy flank includes the sequence TISR and XXX is an epitope sequence. FIG. 6A shows the three-dimensional structure for the polypeptide NGFRFFTISR, having epitope sequence RFF. FIG. 6B shows the three-dimensional structure for the polypeptide NGFTFTTISR, having epitope sequence TFT. FIG. 6C shows the three-dimensional structure for the polypeptide NGFWFRTISR, having epitope sequence WFR.

### Example V

### Structured Aptamer Libraries Using Error Prone PCR

This Example demonstrates generation of a structured aptamer library from a progenitor aptamer that had been previously selected against a highly aptagenic target polypeptide. This Example further demonstrates a higher rate of success using SELEX to identify aptamers from the structured aptamer library compared to a naïve aptamer library. Surprisingly, a progenitor aptamer having affinity for a first set of target epitopes was modified to produce a library of aptamer variants that included individual variants having affinity for epitopes that were not present in the first set of target epitopes.

### Materials and Methods

A progenitor aptamer (B1 aptamer) was subjected to random mutagenesis using techniques set forth in Zaccolo et al., Journal of Molecular Biology 255:589 (1996). The B1 aptamer was subjected to one to six rounds of mutagenic polymerase chain reaction (PCR) using the following master mix: 70.7 ul dH₂O, 10 ul 10×PCR buffer, 10 ul 10 mM MgCl₂, 1 ul 100 uM Forward Primer, 1 ul 100 uM Reverse Primer, 0.8 ul 100 mM dNTP mix (dATP, dGTP, dCTP and dTTP), 2 ul 10 mM 8-Oxo-T-deoxyguanosine-5′-Triphosphate (8-oxo-GTP), 2 ul 10 mM 2′-Deoxy-P-nucleoside-5′-Triphosphate (dPTP) and 0.5 ul Taq Pol (5 U/ul). PCR conditions: (1) 95° C. 3 min., (2) 95° C. 15 sec., (3) 65° C. 30 sec., (4) 72° C. 30 sec., (5) repeat steps 2-4 for 20 cycles. The product of the one to six mutagenic PCR rounds was then subjected to reamplification PCR using the following master mix: 37.35 ul dH₂O, 5 ul 10×PCR buffer, 5 ul 10 mM MgCl₂, 0.5 ul 100 uM Forward Primer, 0.5 ul 100 uM Reverse Primer, 0.4 ul 100 mM dNTP mix (dATP, dGTP, dCTP and dTTP), and 0.5 ul Taq Pol (5 U/ul). The same PCR conditions were used as for the mutagenic PCR and reamplification PCR. A sampling of the reamplification PCR product was subjected to sequencing on an Illumina MiSeq™ Sequencer according to the manufacturer's instructions.

Reamplification PCR products (‘structured aptamer libraries’) were screened using SELEX essentially as set forth in Example I. Naïve aptamer libraries were screened as a control library using the SELEX process in parallel with the structured aptamer libraries. Bait polypeptides used in the screen included one of the following target epitopes: HYH, HHH, HHD, HHM, PHH, HHS, HVE, HHG, NHH, HHV, MHH, SHH, WHH, GHY, HVS, NMH. The target epitopes were selected based on prior observations of binding affinity for the B1 aptamer.

Products from each SELEX round were sequenced on an Illumina MiSeq™ Sequencer according to the manufacturer's instructions and the results evaluated to assess enrichment of aptamers selected from each library. Candidate aptamers identified from the sequencing results were evaluated for ability to bind each of the target epitopes in a triage ELONA. Polypeptide (1 mM) in maleic anhydride immobilization buffer was incubated overnight on a maleic anhydride plate at room temperature and then centrifuged at 500 RPM. The filter was washed 6 times with aptamer binding buffer followed by centrifugation at 500 RPM. Ethanolamine (50 μM) in 100 mM Tris pH 9 was added to the wells and incubated for 2 hours at room temperature followed by centrifugation at 500 RPM. The filter was again washed 6 times with aptamer binding buffer. Aptamers (1 uM) in aptamer binding buffer was incubated for 1 hour at room temperature then centrifuged at 500 RPM. The filter was again washed 6 times with aptamer binding buffer. Detection solution was added to the well and incubated at room temperature followed by centrifugation at 500 RPM. The filter was again washed 6 times with aptamer binding buffer. TMB was added to wells and incubated for 1 minute and 30 seconds before adding 1 N HCl to stop development. The plate was detected by an optical plate reader at a wavelength of 450 nM. The ELONA was also performed to evaluate binding of the B1 aptamer to each of the target epitopes.

### Results and Discussion

A structured aptamer library was constructed by performing one to six consecutive error prone PCRs using a progenitor aptamer (B1 Aptamer) as template and modified dNTPs: 2′-Deoxy-P-nucleoside-5′-Triphosphate (dPTP) and 8-Oxo-2′-deoxyguanosine-5′-Triphosphate (8-oxo-GTP). Random mutagenesis was induced by PCR amplification of the B1 aptamer in the presence of the four natural dNTPs plus the mutagenic analogs, 8-Oxo-dGTP and dPTP. When both mutagenic analogs are used, the rate of mutagenesis is dependent, at least in part, on the number of PCR cycles performed. The dPTP nucleotide is a degenerate base which behaves as a universal pyrimidine and therefore base pairs with both adenine and guanine during PCR resulting primarily in transition mutations (A-to-G and G-to-A). The 8-Oxo-dGTP can mispair with adenine, leading to A-to-C and G-to-T transversion mutations during PCR. Following the error prone PCR, a reamplification reaction was performed using only natural dNTPs to virtually remove the modified analogues.

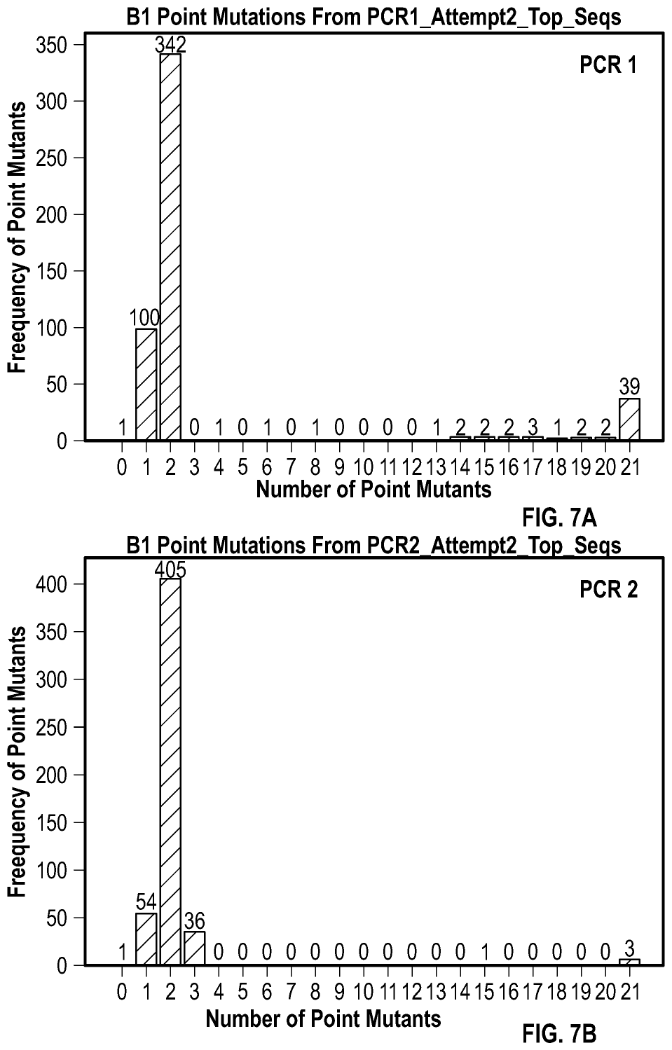

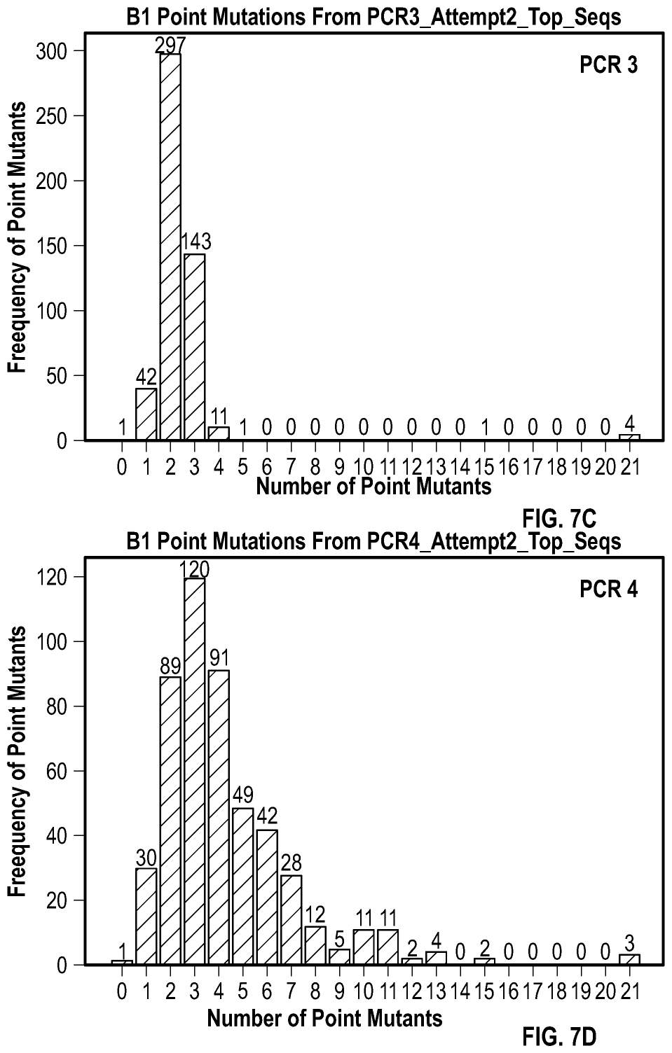

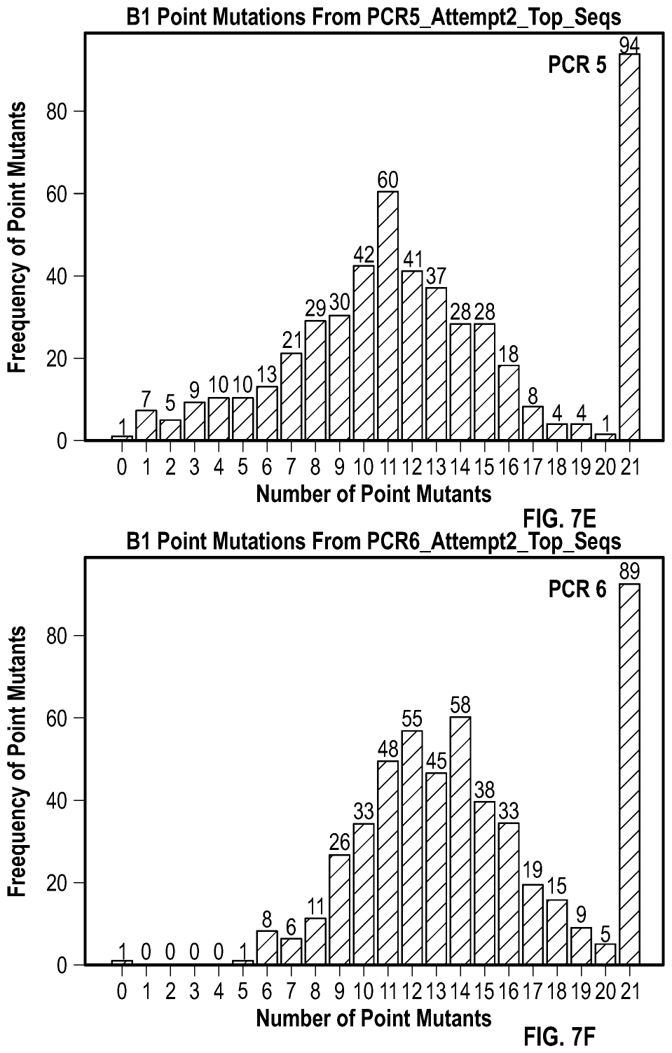

After completion of the mutagenic and reamplification PCRs, the number of point mutants present in each B1 variant was analyzed using next generation sequencing (NGS). FIGS. 7A-7F show the results from each of six rounds of PCR plotted as the number of point mutants observed in an aptamer sequence (X axis) vs. the number of aptamer sequences that have a particular number of point mutants (Y axis). B1 variants with more than 21 point mutations were deemed to be 3′ region duplications and were filtered out. It was found that with every consecutive PCR reaction, the average number of point mutations per B1 variant increased. However, after the fourth PCR reaction, there was a significant increase in point mutations per B1 variant, indicating instead of increasing diversity that the 3′ region was merely being duplicated.

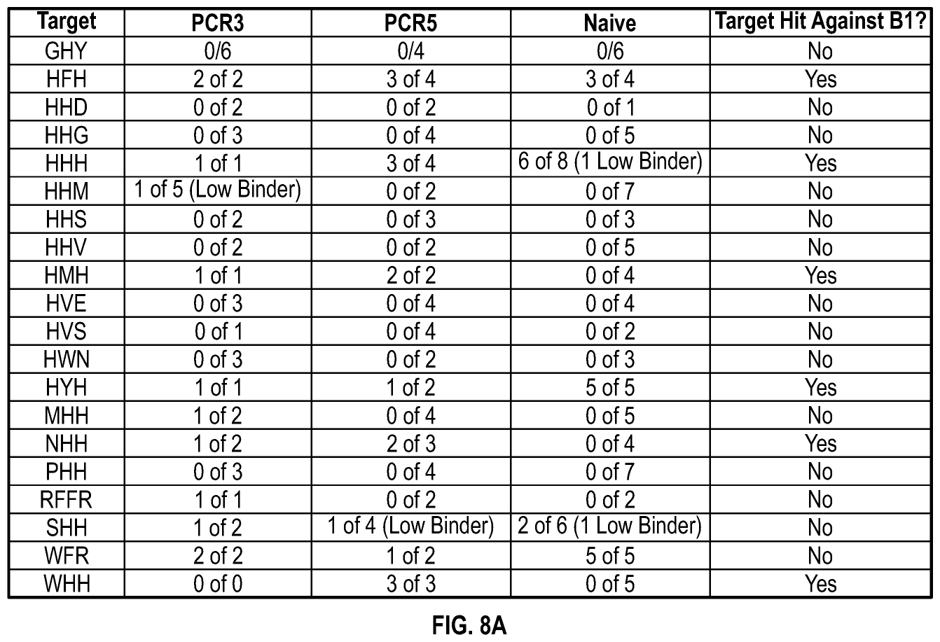

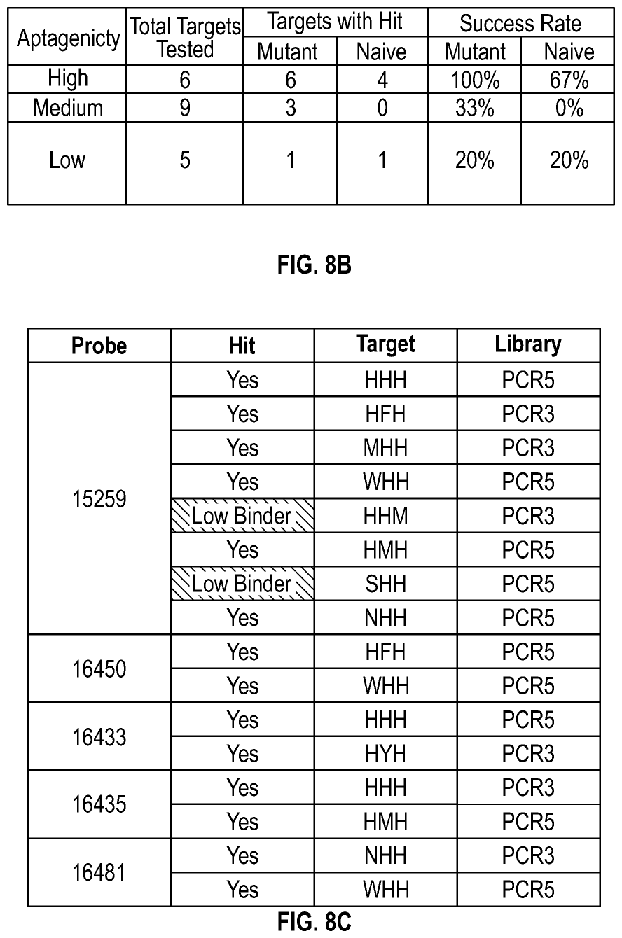

Based on the sequencing results, the libraries of B1 variants from the third PCR (PCR3) and fifth PCR (PCR5) were compared in a SELEX campaign to a standard naïve aptamer library (naïve variants). An enzyme-linked oligonucleotide assay (ELONA) was performed as a triage to quantify the proportion of aptamer hits per epitope target for each library. FIG. 8A tabulates the proportion of aptamer hits per epitope target (“Target”) for each library (“PCR3”, “PCR5” and “Naïve”) and identifies which of the epitope targets did (“Yes”) or did not (No”) bind to the B1 aptamer (“Target Hit Against B1?”). A total of 49 aptamer hits were obtained across all three libraries and the hits encompassed 11 target epitopes. The structured aptamer libraries (PCR3 and PCR5) produced aptamers that recognized targets that were not recognized by products of the naïve library, the targets including HHM, HMH, MHH, NHH, RFFR (SEQ ID NO: 58), and WHH. Thus, the structured libraries appeared to produce a greater variety of desired variants compared to the naïve library. Additionally, the structured libraries produced variants of the B1 aptamer that had better binding to particular targets compared to binding of the progenitor B1 aptamer to the same targets, respectively. Targets that were unique to binding B1 aptamer variants compared the progenitor B1 aptamer included RFFR, SHH and WFR.

The overall success rate for total aptamer variants tested was greater for the structured libraries compared to the naïve library, the success rates being 27% (12/44) for the PCR3 structured library, 26% (16/61) for the PCR5 structured library, and 24% (21/87) for the naïve library. FIG. 8B tabulates the success rate for target epitopes tested according to the aptagenicity of the targets for the B1 aptamer. The results demonstrated a greater enrichment of target-specific aptamers using the structured aptamer libraries than the naïve library. The difference in success rate was most significant for high aptagenicity targets and medium aptagenicity targets.

Five aptamers identified from SELEX screening of the structured aptamer libraries had binding affinity for more than one of the epitope targets tested. The five aptamers and the target epitopes to which they bound are tabulated in FIG. 8C.

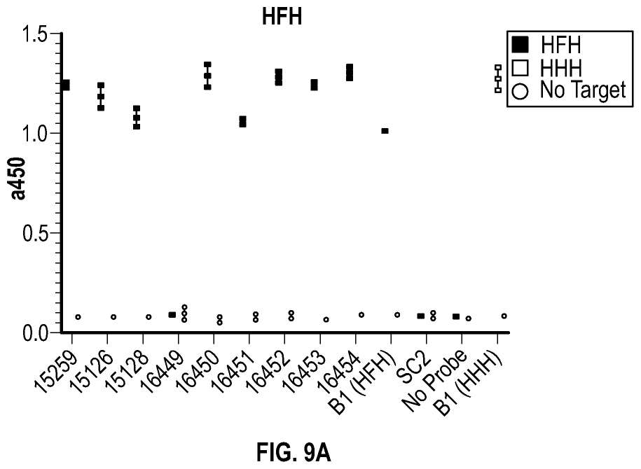

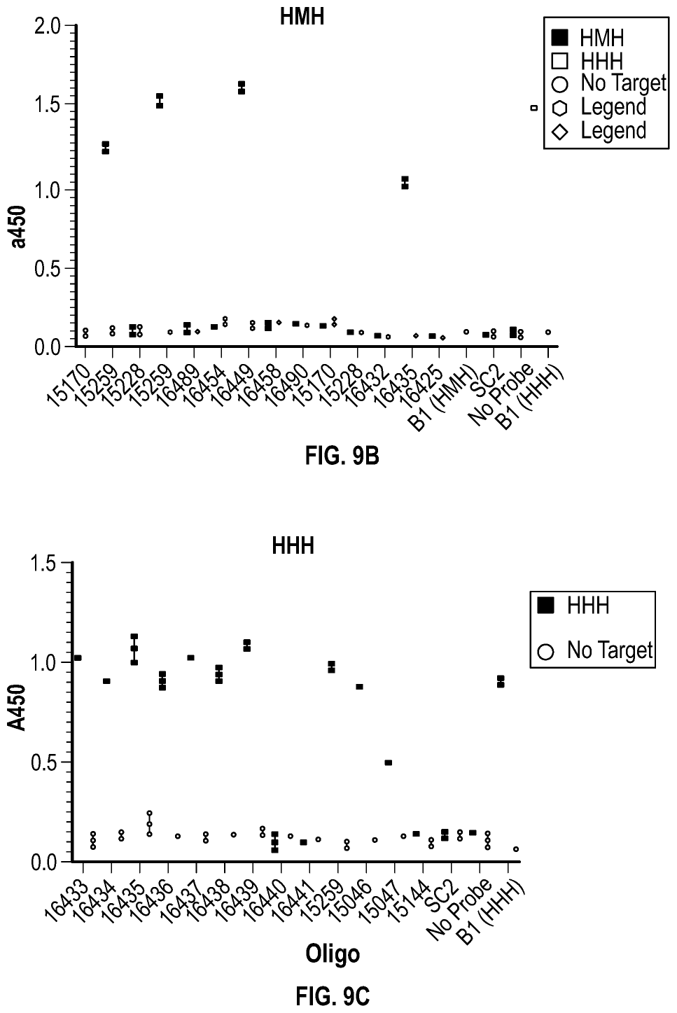

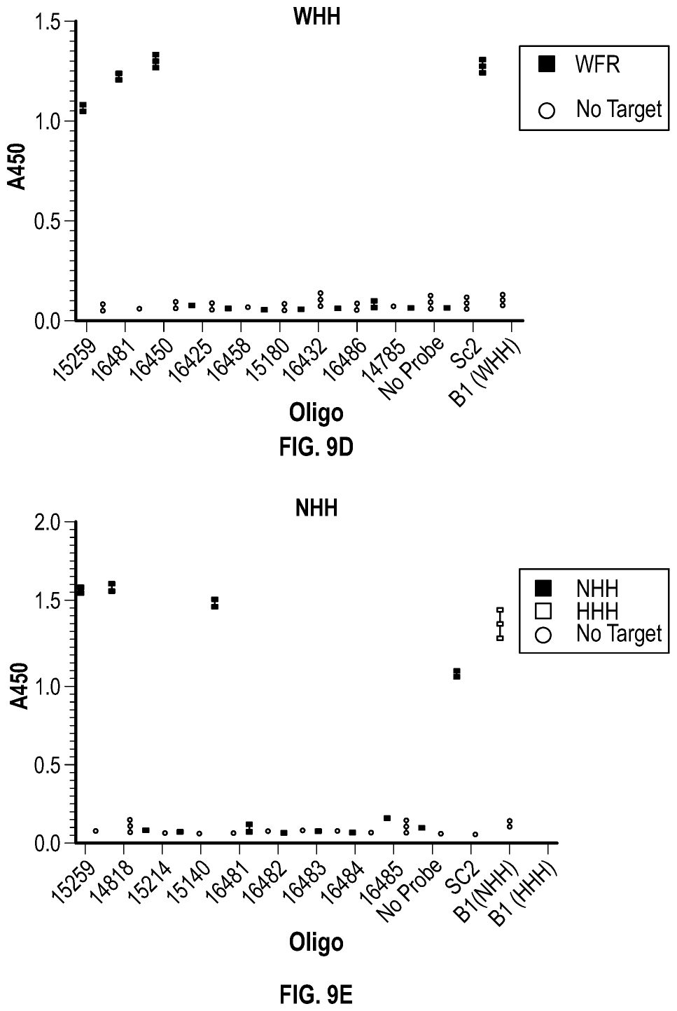

Variant aptamers obtained from the structured aptamer libraries showed similar or better binding properties compared to the progenitor B1 aptamer. As demonstrated by the ELONA results plotted in FIGS. 9A through 9E, for target epitopes that bind to the B1 progenitor aptamer, several B1 variants showed similar or better binding compared to binding of the B1 progenitor aptamer to those targets.

The results of this Example demonstrate that use of structured aptamer libraries in SELEX produced hits against difficult target epitopes. The success rate for use of the structured aptamer libraries was higher compared to use of a naïve aptamer library.

### Example VI

### Aptamer Identification Method

This example demonstrates a process for assessing an aptamer pool for binding to a test polypeptide. The process was able to distinguish binding of known aptamers to test polypeptides and binding could be distinguished in a background of an aptamer library that had not been selected for the test polypeptide.

The binding assessment process was carried out as follows. An aptamer pool was obtained from a SELEX process, performed as set forth in Example I and using a polypeptide target molecule having the HHH tripeptide epitope. The concentration of nucleic acid in the aptamer pool was determined using Tecan Spark ssDNA quantitation (Tecan, Männedorf, Switzerland). Aliquots were removed from the pool and the concentration of nucleic acid in each aliquot was adjusted to 20 nM in AS buffer. Aliquots were incubated with various concentration of biotinylated test polypeptide having the same sequence as the target polypeptide used in the SELEX process. Binding was done using the MagMax™ Express 96 (ThermoFisher) under the following conditions: 20 nM aptamer, variable concentrations of peptide (10 nM-1000 nM) with a 0 nM peptide control. Initially, 3.5 μM Pierce™ Streptavidin Magnetic Beads (ThermoFisher, Cat. No. 88816) were added to each aliquot to bind complexes between aptamers and biotinylated test polypeptide in AS buffer. After one minute of mixing mix beads were collected in the original plate and then transferred to a binding plate. In the binding plate, beads were released; mixed for four and a half minutes with medium mixing then 30 seconds bottom mixing, and this was repeated twice before collecting the beads. The beads were then transferred to a wash plate containing 100 μl of AS buffer. Then beads were released mixed for four minutes with medium mixing then 1 minute bottom mixing, and this was repeated twice before collecting the beads. The beads were then transferred to a collection plate containing 50 μl of AS buffer. Then beads were released, mixed for two minutes with medium mixing, and mixed for one minute with bottom mixing. Beads from each aliquot were incubated with SYBR gold (Thermo Fisher, Waltham MA) and the level of aptamer staining for each bead was detected.

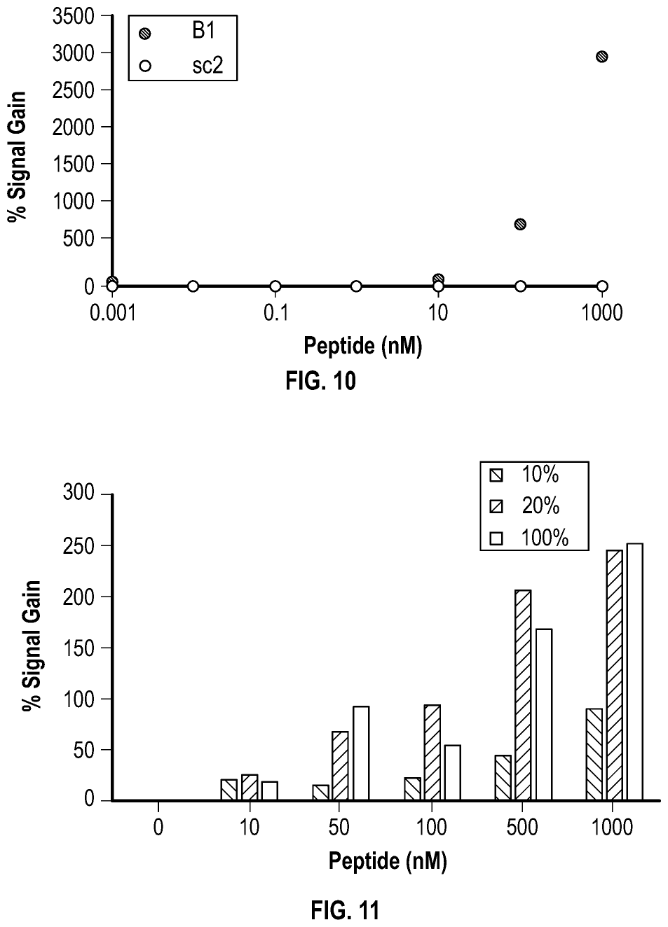

A pilot assay was performed to establish that the assessment process detects a control aptamer and that binding of the aptamer was distinguished from background signal from a random aptamer library. Seven pairs of samples were made, wherein each pair included 50 nM B1 aptamer and 50 nM sc2 aptamer library. The B1 aptamer has known specificity for the HHH tripeptide epitope and the sc2 library has not been selected for specificity to HHH. The samples were incubated with various concentration of biotinylated test polypeptide having the HHH epitope, the resulting complexes were bound to streptavidin beads and aptamers on the beads were stained with SYBR gold and detected as set forth above. A control sample was treated similarly except that biotinylated peptide was not introduced. FIG. 10 shows a plot of percent signal gain for beads from each sample (percent signal gain was normalized to fluorescence detected for beads from the control sample) vs. concentration of biotinylated test polypeptide. The results indicated that signal resulting from binding of B2 aptamer was distinguishable over background signal from the sc2 aptamer library at concentrations of test peptide at and above 100 nM.

A validation assay was performed to establish that the binding assessment process distinguished binding of a control aptamer in a sample containing random library members. Six sample triplets were made, wherein each triplet included 10% B1 aptamer and 90% sc2 aptamer library, 20% B1 aptamer and 80% sc2 aptamer library and 100% B1 aptamer. The samples were incubated with various concentration of biotinylated test polypeptide having the HHH epitope, the resulting complexes were bound to streptavidin beads and aptamers on the beads were stained with SYBR gold and detected as set forth above. A control sample was treated similarly except that biotinylated peptide was not introduced. FIG. 11 shows a plot of percent signal gain for beads from each sample (percent signal gain was normalized to fluorescence detected for beads from the control sample) vs. concentration of biotinylated test polypeptide. The results indicated that signal resulting from binding of B1 aptamer was distinguishable over background signal from the sc2 aptamer library at concentrations of test peptide at and above 10 nM. Moreover, B1 aptamer binding could be distinguished for samples having substantial amounts of background aptamers (i.e. 80% or 90% sc2 aptamer library).

### Example VII

### Making Labeled Aptamer Probes

This example demonstrates methods for making probes each having an origami-based structured nucleic acid particle attached to a plurality of labels and a plurality of aptamers.

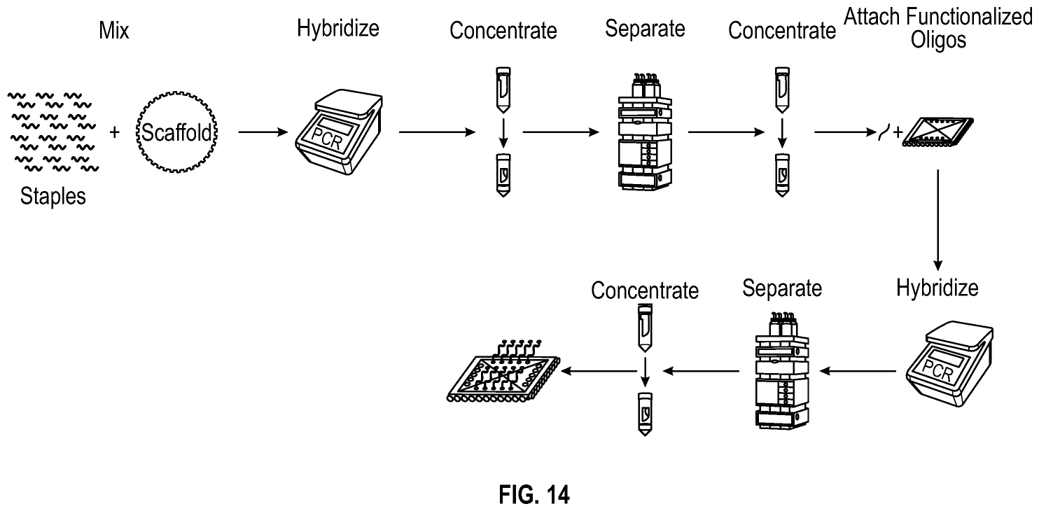

FIG. 14 provides a diagrammatic process flow for a first method of making labeled aptamer probes. In the first step, a 24 ml mixture was formed including 40 nM M13 plasmid, 300 nM nucleic acid staples, 5 mM Tris-Cl, 5 mM NaCl, 1 mM EDTA, 12.5 mM MgCl₂ at pH 8.0. Then the mixture was hybridized in a PCR thermocycler starting at 95° C. for 120 sec followed by temperature ramp starting from 90° C. and ending at 20° C., decreasing one ° C. every minute. The 24 ml reaction was then concentrated to 4 ml volume using Amicon Ultra (Millipore Sigma, Burlington, MA) spin filter with 100 kDa cutoff. The origami tile was separated from excess staples and scaffold using Agilent Bio SEC-5, 5 μm, 2000A, 4.6×300 mm (P/N: 5190-2543) size exclusion column on an Agilent 1200 HPLC system as set forth in Aksel et al. High-density and scalable protein arrays for single-molecule proteomic studies BioRxiv (2022) doi: 10.1101/2022.05.02.490328. Isolated origami tiles were collected in a volume of about 3.5 ml and concentrated to about 800 ng/μ1 using Amicon Ultra (Millipore Sigma, Burlington, MA) spin filter with 100 kDa cutoff. Aliquots were removed from the concentrated sample and run on an agarose gel to evaluate quality of the origami tiles.

The remainder of the concentrated origami tiles were mixed with functionalized nucleic acids. The functionalized nucleic acids included aptamers and dye labeled oligonucleotides. The mixture was then hybridized in a PCR thermocycler starting at 40° C. for 300 sec followed by temperature ramp starting from 40° C. and ending at 20° C., decreasing one ° C. every 30 seconds. The labeled aptamer probes (i.e. origami tiles attached to aptamers and dye-labeled nucleic acids) were separated from excess functionalized nucleic acids using Agilent Bio SEC-5, 5 μm, 2000 Å, 4.6×300 mm (P/N: 5190-2543) size exclusion column on an Agilent 1200 HPLC system as set forth above and labeled aptamer probes were collected in a volume of about 2 ml and concentrated to about 800 ng/μ1 using Amicon Ultra (Millipore Sigma, Burlington, MA) spin filter with 100 kDa cutoff. A further quality test was performed by agarose gel electrophoresis of an aliquot from the concentrated sample.

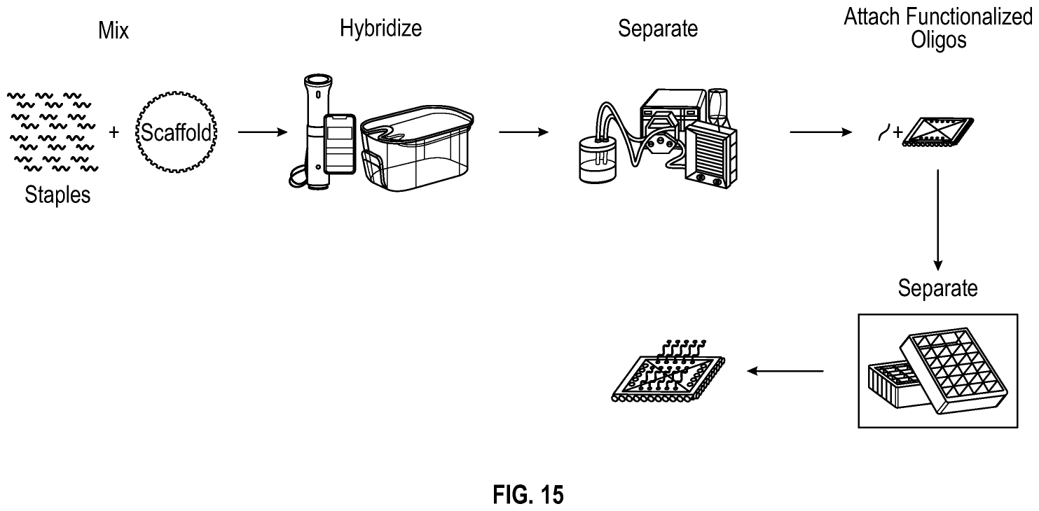

The process set forth above and in FIG. 14 produced pure labeled aptamer probes with an average yield of 40%. However, the process took about 4 days to complete and was limited with respect to the volume of sample that was processed on a per instrument basis. A larger scale process is diagrammed in FIG. 15. In a first step, a mixture was formed containing 40 nM M13 plasmid, 300 nM nucleic acid staples, 5 mM Tris-Cl, 5 mM NaCl, 1 mM EDTA, 12.5 mM MgCl₂ at pH 8.0 in a conical polypropylene tube. The tube was placed in a Precision Cooker from Anova where it was incubated at 70° C. for 10 minutes followed by cooling to room temperature at a rate of 1° C. per 5 minutes. The volume for this step was tested at 24 ml but can be scaled up to liters. The origami tiles were then separated from excess staples and scaffolds using tangential flow filtration on a Sartorius Vivaflow 50R instrument using 100 kDa MWCO Hydrosart cassette. The volume for this step can scale from 24 ml to greater than 1 liter with a flow rate setting at 200 ml/min. A 24 ml sample was purified in about 45 minutes. An advantage of tangential flow filtration is that the retentate contained origami tiles that were sufficiently concentrated for the subsequent step of hybridizing with functionalized nucleic acids in a plate thermomixer to form labeled aptamer probes. The labeled aptamer probes were then separated from functionalized nucleic acids using a filter plate having 100K omega filters (Pall Corp., Westborough, MA). A 24 ml volume of labeled aptamer probes was purified in about 5 hours using the filter plates. An advantage of this approach is that multiple probes can be processed in parallel using a multiwell plate.

Advantages of the scaled-up process set forth in the context of FIG. 14 include (1) labeled aptamer probes can be obtained in about 10 hours (compared to about 4 days for the process of FIG. 13); (2) liters of volume can be conveniently processed; and (3) the yield of labeled aptamer probes is greater than 90% (compared to about 40% yield for the process of FIG. 13).

What is claimed is:

1. A method for producing probes, comprising:
(a) forming a first mixture comprising a plurality of nucleic acid scaffolds and nucleic acid staples, thereby producing structured nucleic acid particles, the structured nucleic acid particles each comprising a nucleic acid scaffold and a plurality of nucleic acid staples folded into a nucleic acid origami;
(b) separating the structured nucleic acid particles from nucleic acid scaffolds and nucleic acid staples of the first mixture;
(c) forming a second mixture comprising a plurality of functionalized nucleic acids and a plurality of the structured nucleic acid particles, thereby producing functionalized structured nucleic acid particles, the functionalized structured nucleic acid particles each comprising a nucleic acid origami hybridized to a functionalized nucleic acid;
(d) separating the functionalized structured nucleic acid particles from functionalized nucleic acids and structured nucleic acid particles of the second mixture;
(e) forming a third mixture comprising affinity reagents and the functionalized structured nucleic acid particles, thereby producing probes, the probes each comprising a structured nucleic acid particle attached to an affinity reagent via a moiety of the functionalized oligonucleotide; and
(f) separating the probes from affinity reagents and functionalized structured nucleic acid particles of the third mixture.

2. The method of claim 1, wherein the first mixture is cooled by a fluid coupled to an oven.

3. The method of claim 1, wherein the separating of step (b) comprises separating nucleic acid scaffolds and nucleic acid staples of the first mixture from the structured nucleic acid particles via tangential flow filtration.

4. The method of claim 1, wherein the functionalized nucleic acids comprise labels.

5. The method of claim 1, wherein the functionalized nucleic acids comprise first chemically reactive moieties, the first chemically reactive moieties being reactive with second chemically reactive moieties on the affinity reagents.

6. The method of claim 1, wherein the functionalized structured nucleic acid particles comprise first chemically reactive moieties, and wherein step (e) comprises reacting the first chemically reactive moieties with second chemically reactive moieties on the affinity reagents, thereby covalently attaching the affinity reagents to the functionalized structured nucleic acid particles.

7. A method for producing probes, comprising:
(a) forming a first mixture comprising a plurality of nucleic acid scaffolds and nucleic acid staples, thereby producing structured nucleic acid particles, the structured nucleic acid particles each comprising a nucleic acid scaffold and a plurality of nucleic acid staples folded into a nucleic acid origami;
(b) separating the structured nucleic acid particles from nucleic acid scaffolds and nucleic acid staples of the first mixture;
(c) forming a second mixture comprising a plurality of functionalized nucleic acids and a plurality of the structured nucleic acid particles, wherein the functionalized nucleic acids comprise affinity reagents, thereby producing probes, the probes each comprising a structured nucleic acid particle attached to an affinity reagent via the functionalized oligonucleotide; and
(d) separating the probes from functionalized nucleic acids and structured nucleic acid particles of the second mixture.

8. The method of claim 7, wherein the first mixture is heated and cooled by contact with a fluid coupled to an oven and wherein the fluid is cooled at a predefined rate.

9. The method of claim 7, wherein the separating of step (b) comprises separating nucleic acid scaffolds and nucleic acid staples of the first mixture from the structured nucleic acid particles via tangential flow filtration.

10. The method of claim 7, wherein the functionalized nucleic acids further comprise nucleic acids attached to labels.

11. A method of selecting at least one affinity reagent, comprising:
(a) contacting a solid-phase analyte with a fluid-phase library of different affinity reagents in a vessel, wherein the solid-phase comprises a particle to which the analyte is attached, wherein the vessel comprises a porous filter that prevents passage of the particle;
(b) passing the fluid-phase library of affinity reagents through the porous filter to separate affinity reagents of the library from the solid-phase analyte;
(c) transferring the solid-phase analyte from the vessel to a second vessel; and
(d) washing the solid-phase analyte in the second vessel to remove further affinity reagents of the library from the solid-phase analyte, whereby at least one of the different affinity reagents remains bound to the solid-phase analyte.

12. The method of claim 11, wherein a plurality of the different affinity reagents remains bound to the solid-phase analyte.

13. The method of claim 12, further comprising steps of:
(e) dissociating the plurality of the different affinity reagents from the solid-phase analyte; and
(f) repeating steps (a)-(d) using the plurality of the different affinity reagents instead of the library, thereby selecting at least one affinity reagent that is bound to the solid-phase analyte.

14. The method of claim 11, wherein the second vessel comprises a second porous filter, wherein the second porous filter prevents passage of the particle.

15. The method of claim 14, wherein step (d) comprises passing a wash solution through the second porous filter to separate affinity reagents of the library from the solid-phase analyte.

16. A method comprising:
(a) providing a library of aptamers;
(b) performing an aptamer selection process, comprising:
(i) contacting the library with solid-phase target analytes, thereby forming solid-phase complexes comprising the target analytes and a subset of the candidate aptamers, and
(ii) separating the subset of candidate aptamers from the library, thereby obtaining a pool of selected aptamers; and
(c) performing a binding assessment process, comprising:
(i) contacting the selected aptamers with test analytes, thereby forming complexes comprising the test analytes and a subset of the selected aptamers,
(ii) separating the subset of selected aptamers from the pool, thereby obtaining a plurality of candidate aptamers, and
(iii) detecting candidate aptamers of the plurality of candidate aptamers.

17. The method of claim 16, wherein the aptamer selection process of step (b), comprises:
(i) contacting the library with solid-phase target analytes in a vessel, wherein the library is in fluid-phase, thereby forming solid-phase complexes comprising the target analytes and a subset of the candidate aptamers, wherein the solid-phase comprises particles to which the target analytes are attached, and wherein the vessel comprises a porous filter that prevents passage of the particles,
(ii) passing the fluid-phase library through the porous filter to separate the solid-phase complexes from the library,
(iii) transferring the solid-phase complexes from the vessel to a second vessel, and
(iv) washing the solid-phase complexes in the second vessel to remove further candidate aptamers of the library from the solid-phase complexes, thereby separating the subset of candidate aptamers from the library, thereby obtaining a pool of selected aptamers.
